(19) United States
(12) Patent Application Publication
(10) Pub. No.: US 2013/0039930 A1
(43) Pub. Date: Feb. 14, 2013

(54) BIOMARKER FOR SENSITIVITY TO THERAPY WITH A NOTCH INHIBITOR

(71) Applicants: Kari Alitalo, Helsinki (FI); Andrey Anisimov, Helsinki (FI); Masahiro Yamamoto, Helsinki (FI)

(72) Inventors: Kari Alitalo, Helsinki (FI); Andrey Anisimov, Helsinki (FI); Masahiro Yamamoto, Helsinki (FI)

(21) Appl. No.: 13/491,993

(22) Filed: Jun. 8, 2012

Related U.S. Application Data
(60) Provisional application No. 61/495,254, filed on Jun. 9, 2011.

(51) Int. Cl. G01N 21/75; C12Q 1/68; A61K 39/395; G06F 19/00; A61K 38/02; A61K 31/713; A61P 35/00; G01N 33/566; A61K 31/7088

(52) U.S. Cl. 424/174.1; 435/7.92; 435/7.95; 435/6.12; 435/6.11; 514/44 A; 514/19.3; 702/19

(57) ABSTRACT

Described herein are materials and methods for identifying subjects that would benefit from Notch-targeted therapy.

### BACKGROUND

Notch signaling regulates several biological processes, such as neural stem cell differentiation, cell differentiation in the intestine and vascular branching (Artavanis-Tsakonas et al., 1999). In the vascular system Notch signals control arteriovenous differentiation and in angiogenesis the tip vs. stalk cell organization in angiogenic sprouting (Phng and Gerhardt, 2009). Inhibition of Notch signaling in xenograft tumor models induces an excess sprouting of endothelial cells, leading to poorly perfused vessels that have fewer pericytes. This decreases oxygen and nutrient supply to the tumor, which results in the suppression of tumor growth (Li et al., 2007; Noguera-Troise et al., 2006; Scehnet et al., 2007).

Vascular endothelial growth factor-C (VEGF-C) has been shown to be one of the key regulators of tumor blood and lymphatic vessel growth (Stacker et al., 2002; Achen et al., 2005; Karpanen et al., 2001; Mattila et al., 2002; He et al., 2002). Vascular endothelial growth factor-C (VEGF-C) expression in tumors has been shown to correlate with the rate of metastasis to lymph nodes in breast, colorectal, gastric, thyroid, lung and prostate cancers (Stacker et al., 2002; Achen et al., 2005). In animal models, VEGF-C overexpression increases lymphatic vessel density (LVD) around and inside tumor, and increases the rate of both lymphatic and lung metastases (Skobe et al., 2001). VEGF-C has also been shown to stimulate angiogenesis in embryos (Lohela et al., 2008), skeletal muscle (Anisimov et al., 2009) and certain tumors (Karpanen et al., 2001; Skobe et al., 2001). Tammela et al. (2008) have reported that Notch inhibition induces VEGFR-3 expression in the blood vessels. Furthermore, they showed that blocking antibodies against VEGFR-3 lead to suppression of angiogenesis. Similarly, inhibition of VEGF-C and VEGF-D with soluble VEGFR-3-Fc was shown to inhibit both angiogenesis and lymphangiogenesis and to suppress lymphatic metastasis (He et al., 2002).

### SUMMARY OF INVENTION

The work described herein demonstrates that VEGF-C is a predictive biomarker and that an elevated level of VEGF-C gene or protein expression indicates sensitivity of a tumor to treatment with an inhibitor of Notch signaling. The level of VEGF-C gene or protein expression can be used as a biomarker for classifying tumors according to their likelihood of responding to treatment with an inhibitor of Notch signaling. Such classification of tumors is useful for treating human subjects in a clinical setting. Such classification also is useful for laboratory research involving experimental animals, e.g., genetically engineered mouse models of cancer.

In one aspect, described herein is a method of screening for a mammalian subject with cancer to identify a subject for whom a Notch-targeted therapy will have efficacy. Such a method comprises the steps of measuring Vascular Endothelial Growth Factor-C (VEGF-C) expression in a biological sample from a mammalian subject with cancer, and identifying or selecting a subject as one for whom a Notch-targeted therapy will have efficacy from the measurement of VEGF-C, wherein elevated VEGF-C expression in the sample identifies the subject as a subject for whom a Notch-targeted therapy will have efficacy.

In some embodiments, the cancer is a solid tumor and the biological sample comprises a tumor biopsy and the VEGF-C is measured in the tumor. In some embodiments, the tumor is a tumor of a tissue or organ selected from the group consisting of colon, rectum, intestine, breast, ovary, lung, stomach, brain, pancreas, ovary, prostate, kidney, liver, and head and neck. In some embodiments, the subject is suffering from a cancer selected from the group consisting of colorectal cancer, breast cancer, lung cancer, gastric cancer, glioblastoma and pancreatic cancer.

In other embodiments, the biological sample includes tumor blood or lymphatic vessel tissue and the VEGF-C is measured in vessel tissue. In still other embodiments, the biological sample includes fluid (e.g., ascites, pleural effusion, cerebrospinal fluid) from the tumor and the VEGF-C is measured in the fluid. In still other embodiments, the biological sample comprises blood, and the VEGF-C is measured in the blood, or in plasma or serum from the blood.

The measuring of VEGF-C can occur after a cancer diagnosis has been made and prior to in initiation of a standard of care cancer therapy (e.g., chemotherapy). In some embodiments, the measuring of VEGF-C occurs after a cancer has become resistant to a standard of care therapy (e.g., chemotherapy). These embodiments are not mutually exclusive. A subject undergoing cancer therapy can be monitored for VEGF-C expression to identify a time point at which VEGF-C becomes elevated and therapy with a Notch inhibitor is indicated according to the invention.

To assess the relative level of VEGF-C (mRNA or protein) expression, the level of VEGF-C expression in a cancer tissue sample can be subjected to one or more of various comparisons. In general, it can be compared to: (a) VEGF-C mRNA or protein expression level(s) in normal tissue from the organ in which the cancer originated; (b) VEGF-C mRNA or protein expression levels in a collection of comparable cancer tissue samples; (c) VEGF-C mRNA or protein expression level in a collection of normal tissue samples; or (d) VEGF-C mRNA or protein expression level in an arbitrary standard. In some embodiments, the screening methods described herein comprises comparing the expression of VEGF-C in the tumor (or vessel tissue or tumor fluid) to the level of VEGF-C expression in healthy tissue of the same type as the tumor, wherein elevated VEGF-C expression in the tumor (or vessel tissue or tumor fluid) to compared to the healthy tissue identifies the subject as a subject for whom Notch-targeted therapy will have efficacy.

The identifying or selecting step of the screening methods described herein optionally comprises comparing the measurement of VEGF-C to a reference measurement of VEGF-C, and scoring the VEGF-C measurement from the sample as elevated based on statistical analysis or a ratio relative to the reference measurement. In some embodiments, the reference measurement comprises at least one of the following (a) a measurement of VEGF-C from healthy tissue of the subject of the same tissue type as the sample; (b) a database containing multiple VEGF-C measurements from healthy or cancerous tissues from other subjects; or (c) a reference value calculated from multiple VEGF-C measurements from healthy or cancerous tissues from other subjects, optionally further including statistical distribution information for the multiple measurements, such as standard deviation.

In some embodiments, a VEGF-C measurement of at least 1.0 standard deviation greater than a median VEGF-C measurement in corresponding healthy tissue is scored as elevated VEGF-C expression. In other embodiments, a VEGF-C measurement that is statistically significantly greater than VEGF-C measurements in corresponding healthy tissue, with a p-value less than 0.1, or less than 0.05, or less than 0.01, or less than 0.005, or less than 0.001 is scored as elevated VEGF-C expression.

In some variations, the level of VEGF-C in a subject is compared to a predetermined “cut-off” concentration of VEGF-C that has been determined from observations to represent an elevated measure of VEGF-C that (when equaled or exceeded) is predictive of efficacy of Notch-targeted therapy. Determination of a suitable cut-off is made using, e.g., statistical analysis of VEGF-C protein or mRNA concentration data collected from multiple healthy and/or cancer patients (including subjects in whom VEGF-C has been measured and for whom Notch-targeted therapy has been employed and monitored for efficacy). If a “cut-off” value is employed, the cut-off concentration preferably is statistically determined to have optimal discriminating value for subjects who benefit from the Notch-targeted therapy (e.g., to have maximum sensitivity and specificity). It will be appreciated that statistical analysis of a dataset will permit clinicians to make informed decisions based on concentrations other than the optimal discriminating concentration (e.g., above or below the optimal discriminating concentration). For example, using receiver-operating-characteristic curves, or using other statistical summaries of VEGF-C concentration and treatment outcome data collected according to the invention, the practitioner is capable of selecting a cut-off VEGF-C concentration having a desired level of sensitivity or specificity for predicting efficacy of Notch-targeted therapy. Considerations regarding the probability of success of Notch-targeted therapy based on VEGF-C measurement, versus the probability of success of alternative therapies based on any available clinical data, can guide the selection of an appropriate cut-off concentration of VEGF-C for making a treatment decision.

In some embodiments, the screening methods described herein further comprise measuring the expression of at least one gene selected from the group consisting of HES1, HES4, HES5, HESL, HEY-2, DTX1, MYC, NRARP, PTCRA, SHQ1, and HeyL (hairy/enhancer-of-split related with YRPW motif-like) in the biological sample, wherein elevated VEGF-C expression and elevated expression of the second gene in the biological sample correlate with a pathological phenotype (i.e., cancer). Standard multivariate statistical analysis tools are used to optimize the predictive value of VEGF-C in combination with one or more of these additional markers.

Any available technique can be used for measuring VEGF-C expression, including direct and indirect techniques. For example, in one variation, the measuring comprises measuring VEGF-C protein in the biological sample. Exemplary techniques for measuring amounts or concentrations of VEGF-C protein in a sample are immunological techniques that involve use of a polyclonal or monoclonal antibody that specifically binds VEGF-C, or use of a VEGF-C-binding fragment of such an antibody. For example, the measuring comprises contacting the biological sample with a VEGF-C antibody (or with a polypeptide comprising an extracellular domain (ECD) fragment of VEGFR-3 that binds VEGF-C) or antigen-binding fragment thereof. Quantification of the amount of bound antibody (e.g., using a label or second, labeled antibody) provides a measurement of VEGF-C protein expressed in the sample. Immunoassays such as radioimmunoassay, immunoradiometric assay (labeled antibody), or an enzyme-linked immunosorbent assay (ELISA) are contemplated.

In another variation, the measuring comprises measuring VEGF-C mRNA in the biological sample. Any available assay for measuring specific oligonucleotides is suitable. One technique for measuring VEGF-C mRNA comprises in situ hybridization to measure VEGF-C mRNA in the biological sample. Other techniques involve steps of isolating mRNA from the biological sample and measuring VEGF-C mRNA in the isolated mRNA, for example, by Northern hybridization procedures. In still another variation, quantitative reverse transcriptase polymerase chain reaction (PCR), real-time PCR, or other PCR techniques are employed to quantitatively amplify VEGF-C mRNA (relative to control samples) to provide a quantitative measurement of VEGF-C mRNA in the colon tissue.

The screening methods described herein may optionally comprise the step of prescribing for or administering to the subject identified as having elevated VEGF-C expression in the biological sample a composition comprising a molecule that suppresses expression of downstream activity of Notch (i.e., a Notch inhibitor).

In another aspect, described herein is a method of treatment comprising obtaining a tumor or tumor biopsy from a mammalian subject, determining that the tumor or tumor biopsy has elevated expression of VEGF-C, and prescribing for or administering to the subject a composition comprising a molecule that suppresses expression or signaling activity of Notch (“Notch inhibitor”).

In some variations, the determining step comprises ordering a laboratory test that measures VEGF-C in the tumor or tumor biopsy and learning the measurement from a report from the laboratory. In other variations, the determining step comprises measuring VEGF-C mRNA or VEGF-C protein in the tumor or tumor biopsy.

Aspects of the invention that are described herein as methods (especially methods that involve treatment) can alternatively be described as (medical) uses of reagents or therapeutics. For example, in one variation, the invention is a use of a composition that comprises a molecule that suppresses expression or activity of Notch for the treatment of cancer in a subject identified with cancer and identified with elevated VEGF-C expression in the cancer (wherein the subject is identified as having elevated VEGF-C expression in the cancer by a method described herein).

Notch inhibitors contemplated for use in the methods (or uses) described herein include, but are not limited to, (a) an antibody that binds a Notch protein and inhibits ligand-mediated stimulation of Notch signaling; (b) a soluble polypeptide that comprises an ECD fragment of a Notch polypeptide that binds a Notch ligand and inhibits the ligand from stimulation of Notch signaling; (c) an antisense or interfering nucleic acid (e.g., antisense oligonucleotide; micro-RNA, short interfering RNA) that inhibits Notch expression; (d) an antibody that binds a Notch ligand protein and inhibits the ligand from stimulation of Notch signaling; (e) a soluble notch ligand polypeptide that comprises an ECD fragment of a Notch ligand that binds Notch and inhibits stimulation of Notch by ligand expressed by a cell; (f) an antisense or interfering nucleic acid (e.g., antisense oligonucleotide; micro-RNA, short interfering RNA) that inhibits expression of a Notch ligand; (g) a small molecule that inhibits Notch expression or signaling; (h) a molecule that inhibits proteolytic cleavage-activation of Notch; and (i) a molecule that inhibits Notch NICD peptide from binding core binding factor-1 (CBF-1) or activating transcription of one or more genes selected from HES, Myc, and p21.

In some embodiments, the Notch inhibitor is selected from the group consisting of (a) an antibody that binds a Notch protein and inhibits delta-like ligand 4 (Dll4) stimulation of Notch signaling; (b) a soluble polypeptide that comprises an ECD fragment of a Notch polypeptide that binds Dll4 and inhibits Dll4 stimulation of Notch signaling; (c) an antisense or interfering nucleic acid (e.g., antisense oligonucleotide; micro-RNA, short interfering RNA) that inhibits Notch expression; (d) an antibody that binds a Dll4 protein and inhibits Dll4 stimulation of Notch signaling; (e) a soluble Dll4 polyeptide that comprises an ECD fragment of Dll4 that binds Notch and inhibits stimulation of Notch by cellular Dll4; and (f) an antisense or interfering nucleic acid (e.g., antisense oligonucleotide; micro-RNA, short interfering RNA) that inhibits Dll4 expression.

In other embodiments, the Notch inhibitor comprises the ECD fragment of the Notch polypeptide or Notch ligand fused to an immunoglobulin constant domain fragment (Fc). Notch4 and Notch 1 polypeptides are preferred, but all Notch polypeptides are contemplated.

In still other embodiments, the Notch inhibitor is selected from the group consisting of: inhibitors of the TNFα converting enzymes (TACE inhibitors), such as ADAM10 and ADAM17; inhibitors of gamma-secretase; and inhibitors set forth in Table 1A.

In the treatment methods (or uses) described herein, the methods optionally comprises administering a standard or care therapeutic to the subject in combination with the Notch inhibitor. With respect to any combination treatment or therapy regimens described herein, the Notch inhibitor composition can be administered simultaneously with the other active agents, which may be in admixture with the Notch inhibitor, or may be in a separate composition. Each composition preferably includes a pharmaceutically acceptable diluent, adjuvant, or carrier. When the agents are separately administered, they may be administered in any order.

Another aspect of the invention is a system for identifying a human subject with cancer as a subject for whom a Notch-targeted therapy will have efficacy, the system comprising: (a) at least one processor; (b) at least one computer-readable medium; (c) a database operatively coupled to a computer-readable medium of the system and containing population information correlating the measurement of VEGF-C expression and efficacy of Notch-targeted therapy data in a population of humans with cancer; (d) a measurement tool that receives an input about the human subject and generates information from the input about the measurement of VEGF-C expression from the human subject; and (e) an analysis tool or routine that: (i) is operatively coupled to the database and the measurement tool, (ii) is stored on a computer-readable medium of the system, (iii) is adapted to be executed on a processor of the system, to compare the information about the human subject with the population information in the database and generate a conclusion with respect to a likelihood of efficacy of Notch-targeted therapy in the human subject.

As described below in detail, some variations of a system of the invention include a medical protocol database operatively connected to a computer-readable medium of the system and containing information correlating the measurement of VEGF-C expression (and any of the one or more additional parameters described herein) and medical protocols for treating human subjects with cancer. Such a system further includes a medical protocol tool or routine to compare or correlate the conclusion obtain from the analysis routine and the medical protocol database, and generate a protocol report with respect to the probability that one or more medical protocols will achieve a therapeutic goal, or a protocol report assessing the relative merits of different protocols for the subject's cancer.

The foregoing paragraphs are not intended to define every aspect of the invention, and additional aspects are described in other sections, such as the Detailed Description. The entire document is intended to be related as a unified disclosure, and it should be understood that all combinations of features described herein are contemplated, even if the combination of features are not found together in the same sentence, or paragraph, or section of this document. Where protein therapy is described, embodiments involving polynucleotide therapy (using polynucleotides that encode the protein) are specifically contemplated, and the reverse also is true.

In addition to the foregoing, the invention includes, as an additional aspect, all embodiments of the invention narrower in scope in any way than the variations defined by specific paragraphs above. For example, certain aspects of the invention that are described as a genus, and it should be understood that every member of a genus is, individually, an aspect of the invention. Although the applicant(s) invented the full scope of the invention described herein, the applicants do not intend to claim subject matter described in the prior art work of others. Therefore, in the event that statutory prior art within the scope of a claim is brought to the attention of the applicants by a Patent Office or other entity or individual, the applicant(s) reserve the right to exercise amendment rights under applicable patent laws to redefine the subject matter of such a claim to specifically exclude such statutory prior art or obvious variations of statutory prior art from the scope of such a claim. Variations of the invention defined by such amended claims also are intended as aspects of the invention.

### BRIEF DESCRIPTION OF THE DRAWINGS

The patent or application file contains at least one drawing executed in color and one color photograph. Copies of this patent or patent application publication with color drawing(s) will be provided by the Office upon request and payment of the necessary fee.

FIG. 1. Expression and function of Dll4-Fc. (A) Map of the AAV9-Dll4-Fc vector. (B) Structure of the Dll4-Fc as a dimeric fusion protein of the ECD of mouse Dll4 and Fc region of human immunoglobulin (hFc). (C) Dll4-Fc in the supernatants of AAV9-transfected 293T cells was analyzed by metabolic labeling, immunoprecipitation with Protein A Sepharose and autoradiography after collecting with protein Sepharose from the supernatant of the virus-infected cells. (D) Human dermal microvascular endothelial cells (HDMECs) on glass coverslips stained with Dll4-Fc (red) on ice. After fixation with paraformaldehyde (PFA) and permeabilzation, the cells were stained for Prox-1 (Green) and Hoechst (Blue). Human immunoglobulin G1 (hIgG) was used as a negative control (Red). Note Dll4-Fc in both the Prox-1-positive lymphatic vascular endothelial cells (LECs) and Prox-1-negative cells. (E) Effect of Dll4-Fc on Notch downstream gene expression. HDMECs were treated with 10 μg/mL Dll4-Fc or 10 μg/mL DAPT for 24 hours, and the expression of Hes-1, Hey-2 and NRARP were quantified by qPCR and normalized for the expression of β-actin. The experiment was repeated 4 times. (F) The cells used in (E) were stained for NICD lymphatic vascular endothelial cells (LECs). Prox-1-positive LECs are indicated by arrowheads. (G) Quantification of NICD fluorescence intensity. **:p<0.01. *: p<0.05. Scale bar in D, 20 μm; in E, 20 μm. ITR: inverted terminal repeat, pCMV: cytomegalovirus promoter, SP: signal peptide, DSL: Delta, Serrated, LAG2 domain, WPRE: Woodchuck hepatitis virus post-transcriptional regulatory element, mDll4 ECD, mouse Dll4 extracellular domain.

FIG. 2. Effects of Dll4-Fc on endothelial cells in culture. (A) Blood vascular endothelial cell (BEC) spheroid sprouting assay. PECAM-1 stain (green). Arrows indicate sprouts. (B) Quantification of the sprout number per spheroid. The combination of Dll4-Fc and VEGF-C increases sprouting significantly more than Dll4-Fc or VEGF-C alone. (C) Sprout-like BEC structures in LECs island. A 1:1 mixture of LECs and BECs was treated for 48 hours with the combination of protein indicated in the figure and stained for PECAM-1 (green) and podoplanin (red). Arrowheads indicate sprout-like structures. (D) Quantification of the sprout-like structures. **: p<0.01, *: p<0.05, NS: not significant. Scale bars in A and C, 100 μm.

FIG. 3. VEGF-C production and sensitivity to Dll4-Fc in cultured tumor cells. (A) Immunocytochemistry for VEGF-C. Almost all the transfected (SW-VEGF-C) cells were positive for VEGF-C. (B) VEGF-C in culture medium, analyzed by metabolic labeling, precipitation with VEGFR-3(D1-3)-Fc and autoradiography. (C) Ligand bioassay using BaF3/VEGFR-3 cells and supernatants from SW-Luc and the SW-VEGF-C cells. (D) Density of cultured SW-Luc and SW-VEGF-C cells treated for 72 hrs with various concentration of Dll4-Fc. The relative optical density values from the MTT assay were compared. Even at the highest Dll4-Fc concentration (50 μg/mL), the proliferation ratio was not affected. Scale bar 50 μm.

FIG. 4. The effects of Dll4-Fc on the growth of SW480R tumors. (A) SW-Luc tumors. The Dll4-Fc treated group tends to grow slower than HSA group, but there was no statistically significant difference. (HSA vs Dll4-Fc: 387±32 vs 287±28 mm³). (B) SW-VEGF-C tumors (HSA vs Dll4-Fc: 484±90 vs 179±34 mm³). (C) SW-VEGF-C tumors. At 7 day after tumor implantation (arrow), 1×10¹² vg AAV9-Dll4-Fc or -HSA were injected intravenously (HSA vs Dll4-Fc: 527±128 vs 216±59 mm³). N=10 in all experiments **: p<0.01, *: p<0.05, NS: not significant.

FIG. 5. The effects of Dll4-Fc on the vasculature of the SW480R tumors. (A)

Immunohistochemistry for endomucin (green), LYVE-1 (red) and Hoechst (blue) staining in tumor sections. (B) Quantification of endomucin and Lyve-1 positive areas. **:p<0.01, *:p<0.05, NS: not significant. Scale bar 100 μm.

FIG. 6. Dll4-Fc suppresses growth of tumors expressing high levels of endogenous VEGF-C. (A) VEGF-C mRNA levels in the indicated tumor cell lines analyzed by qRT-PCR. (B) Effects of Dll4-Fc on the proliferation LNM35 and SK-MeI-103 cells in culture. Dll4-Fc did not affect the proliferation. (C) The effect of Dll4-Fc on the growth of LNM35 and SK-MeI-103 tumors. (D) Endomucin staining of LNM35 and SK-MeI-103 tumors. More sprouts were associated with Dll4-Fc treatment (arrows). (E) Quantification of endomucin positive areas in LNM35 and SK-MeI-103 tumors from (D). **:p<0.01. Scale bar 50 μm.

FIG. 7. Neutralization of VEGF-C/D attenuates the inhibition by Dll4-Fc. (A) The mice were injected by AAV9-Dll4-Fc or AAV9-HSA, followed by subcutaneous implantation of 5×10⁶ VEGFR-3(D1-3)-Fc expressing or control retrovirus-transfected SK-MeI-103 cells. *: p<0.01, **: p<0.05. (B) Endomucin staining of the tumors. (C) Quantification of endomucin positive area. Although AAV9-Dll4-Fc increased vascular density in the SK-control tumors, it did not significantly increase vascular density in the SK− VEGFR-3(D1-3)-Fc tumors. *: p<0.05, **: p<0.01, NS: not significant. Scale bar 100 μm.

FIG. 8. Dll4-Fc expression analysis. Dll4-Fc concentration in serum in the experiment of FIG. 3. The AAV9-Dll4-Fc or AAV9-HSA were injected intravenously 14 days and 1 day before tumor implantation. Black arrows: Vector injection, white arrow: Tumor implantation.

FIG. 9. Dll4-Fc induced hypersprouting and VEGFR-3 expression in tumors. (A) High magnification fluorescent imaging of blood vessels in the SW480 tumors. Endomucin staining. Scale bar: 10 μm. Arrows indicate more sprouts in Dll4-Fc treated SW-VEGF-C tumors. (B) Double staining of endomucin and VEGFR-3 in the AAV9-Dll4-Fc and AAV9-HSA-treated SK-MeI-103 tumors. Scale bar: 20 μm. (C) Quantification of VEGFR-3 staining intensity in the blood vessels. **: p<0.01.

FIG. 10. The effects of Dll4-Fc in the liver. (A) Macroscopic images of livers treated with AAV9-HSA and AAV9-Dll4-Fc. Note that the surface of the Dll4-Fc-treated liver is irregular (arrowheads). (B) Microscopic images of the liver. The sinusoids of the Dll4-Fc-treated livers were distended and the hepatocytes were damaged, especially at the centrilobular area (CV). Scale bar in A, 10 mm. in B, 200 μm.

FIG. 11. Establishment of the VEGFR-3(D1-3)-Fc expressing SK-MeI-103 cell line. (A) Map of the pMXs-VEGFR-3(D1-3)-Fc vector for retrovirus production. (B) Structure of the VEGFR-3(D1-3)-Fc protein. (C) Expression of VEGFR-3(D1-3)-Fc in the culture medium. (D) Suppression of VEGF-C-induced BaF3/VEGFR-3 cell proliferation by the supernatant from the SK-VEGFR-3(D1-3)-Fc cells. LTR: long terminal repeat, Ψ: Ψ sequence for packaging. SP: signal peptide.

FIG. 12 is an exemplary system comprising exemplary computer components.

FIG. 13 is a flow chart depicting system components and operation.

FIG. 14 is another flow chart depicting system components and operation.

### DETAILED DESCRIPTION OF THE INVENTION

The results provided herein regarding the induction of a hypersprouting phenotype by Notch inhibition and the simultaneous VEGFR-3 upregulation in the tip cells of the angiogenic sprouts suggests that VEGF-C is a major driver of this phenotype and in part responsible for the poor perfusion of tumor vessels that leads to decreased tumor growth.

We have tested this concept in the present work by using tumor cells expressing very little or high levels of VEGF-C and Notch inhibition by using a soluble Dll4 (Dll4-Fc) produced in vivo via transfection by adeno-associated virus vector. Our results indicate that the therapeutic benefit of Notch inhibition is best in the VEGF-C overexpressing tumors, which have been shown to have a poor prognosis.

Notch Pathway

The Notch signaling pathway comprises a family of transmembrane receptors, their ligands, negative and positive modifiers, and transcription factors (Jarriault et al., 1995; Schweisguth, 2004). To date, four mammalian receptors (Notch1, Notch2, Notch3 and Notch4) and at least five ligands (Deltal, Delta3, Delta4, Jagged1 and Jagged2) have been identified. Sequence accession numbers and identification of the ECD of human Notch receptors and ligands are provided below.

| | Swiss-Prot | ECD amino acid | |
|---|---|---|---|
| Name | Accession Nos. | fragment | SEQ ID NOs: |
| Human Notch1 | P46531 | 19-1735 | 3 and 4 |
| Human Notch2 | Q04721 | 26-1677 | 5 and 6 |
| Human Notch3 | Q9UM47 | 40-1643 | 7 and 8 |
| Human Notch4 | Q99466 | 24-1447 | 9 and 10 |
| Human Delta1 | O00548 | 18-545 | 11 and 12 |
| Human Delta3 | Q9NYJ7 | 27-492 | 13 and 14 |
| Human Delta4 | Q9NR61 | 27-529 | 15 and 16 |
| Human Jagged1 | P78504 | 34-1067 | 17 and 18 |
| Human Jagged2 | Q9Y219 | 27-1080 | 19 and 20 |

Nonhuman orthologs of these proteins are known and are reported in databases and literature.

Binding of the ligand renders the Notch receptor susceptible to metalloprotease- and γ-secretase-mediated proteolytic cleavage, which in turn results in the release of the Notch intracellular domain (ICN) from the plasma membrane and its subsequent translocation into the nucleus. Once there, ICN associates with DNA-binding protein recombination signal-binding protein JKCBF1/Su (H)/Lag-1 (Rbpj) and mastermind-like (MAML) protein, which recruit additional factors with histone acetylase activity, such as p300 and p300/CREB-binding protein-associated factor. These proteins form a heteromeric complex that mediate transcription of target genes, including basic helix-loop-helix transcription factors of the hairy and enhancer of split (Hes) family and the Hes-related repressor protein (Hey) family (Ilagan et al., 2007).

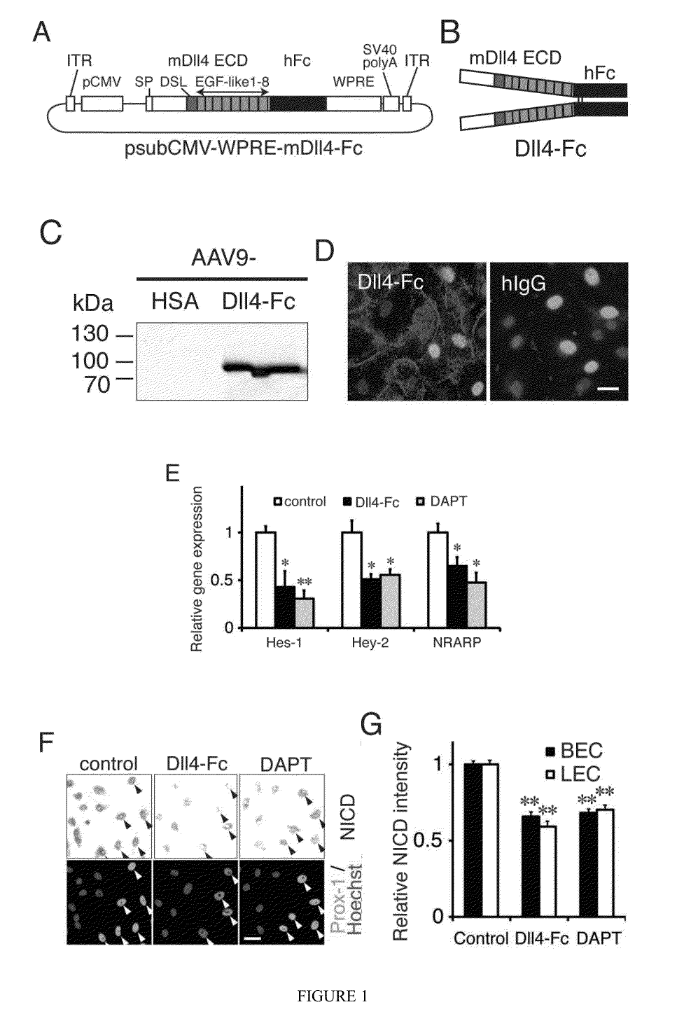

Inhibition of Notch signaling can be achieved at many different levels. Agents targeted to some of these levels have been described (Rizzo et al., 2008, incorporated by reference. See, e.g., FIG. 1 and related text.) and are described elsewhere herein.

Methods of Screening/Identifying Subjects for Treatment with a Notch Inhibitor

The demonstration that colon cancer cells comprising elevated levels of VEGF-C are more sensitive to Notch inhibitors indicates that detection of VEGF-C polynucleotides and polypeptides (including variants thereof) are useful for the identification of subjects that would benefit from treatment with a Notch inhibitor. Therefore, aspects of the present invention are directed to methods of screening a mammalian subject with cancer to identify subjects for whom a Notch-targeted therapy will have efficacy. Such methods including measuring VEGF-C expression in a biological sample from a mammalian subject with cancer and identifying an elevated VEGF-C expression in the sample, wherein an elevated level of VEGF-C expression in the sample identifies the subject as a subject for whom a Notch-targeted therapy will have efficacy.

The methods described herein involve providing a sample from a cancerous tissue in a mammal, e.g., a laboratory mouse or a human patient, so the level of VEGF-C gene (or protein) expression can be determined in the sample. The form of the sample and the method of obtaining the sample will depend on the type of cancerous tissue involved.

In some embodiments, the biological sample comprises cancerous tissue. Cancerous tissue from a solid tumor (e.g., a carcinoma, sarcoma, glioma or lymphoma) can be obtained by using conventional tumor biopsy instruments and procedures. Endoscopic biopsy, excisional biopsy, incisional biopsy, fine needle biopsy, punch biopsy, shave biopsy and skin biopsy are examples of recognized medical procedures that can be used by those of skill in the art to obtain tumor samples for use in practicing the methods described herein. Samples of cancerous lesions may be obtained by resection, bronchoscopy, fine needle aspiration, bronchial brushings, or from sputum, pleural fluid or blood. The expression of VEGF-C (mRNA and protein) can also be detected from cancer or tumor tissue or from other body samples such as urine, sputum, serum or plasma. In some variations, a tumor or tumor biopsy is obtained from a subject and fluid from the tumor is removed, e.g., by centrifugation, and the tumor fluid is used as the sample for measuring VEGF-C.

In some embodiments, the subject is suffering from a cancer selected from the group consisting of colorectal cancer, breast cancer, lung cancer, gastric cancer, glioblastoma and pancreatic cancer.

In practicing the methods described herein, determining the level of VEGF-C expression can be performed by any suitable method, e.g., mRNA-based methods or protein-based methods. Various methods of determining the level of expression of a gene of interest are known in the art.

The measuring of VEGF-C can occur after a cancer diagnosis has been made and prior to in initiation of a standard of care cancer therapy (e.g., chemotherapy). In some embodiments, the measuring of VEGF-C occurs after a cancer has become resistant to a standard of care therapy (e.g., chemotherapy).

VEGF-C (SEQ ID NOs: 1 and 2) is originally expressed as a larger precursor protein, prepro-VEGF-C, having extensive amino- and carboxy-terminal peptide sequences flanking a VHD, with the C-terminal peptide containing tandemly repeated cysteine residues in a motif typical of Balbiani ring 3 protein. The prepro-VEGF-C polypeptide is processed in multiple stages to produce a mature and most active VEGF-C polypeptide CAME VEGF-C) of about 21-23 kD (as assessed by SDS-PAGE under reducing conditions). Such processing includes cleavage of a signal peptide (approximately residues 1-31 of SEQ ID NO: 2); cleavage of a carboxyl-terminal peptide (approximately residues 228-419 of SEQ ID NO: 2) to produce a partially-processed form of about 29 kD; and cleavage (apparently extracellularly) of an amino-terminal peptide (approximately residues 32-102 of SEQ ID NO: 2) to produce a fully-processed mature form of about 21-23 kD (approximately residues 103-227 of SEQ ID NO: 2). Experimental evidence demonstrates that partially-processed forms of VEGF-C (e.g., the 29 kD form) are able to bind the Flt4 (VEGFR-3) receptor, whereas high affinity binding to VEGFR-2 occurs only with the fully processed forms of VEGF-C. Moreover, it has been demonstrated that amino acids 103-227 of SEQ ID NO: 2 are not all critical for maintaining VEGF-C functions. For example, a polypeptide consisting of amino acids 112-215 (and lacking residues 103-111 and 216-227) of SEQ ID NO: 2 retains the ability to bind and stimulate VEGF-C receptors. The cysteine residue at position 156 has been shown to be important for VEGFR-2 binding ability. It appears that VEGF-C polypeptides naturally associate as non-disulfide linked dimers.

In some embodiments, the methods described herein are practiced through the detection of a VEGF-C protein. In general, methods for detecting a VEGF-C protein can comprise contacting a biological sample with a compound that binds to and forms a complex with the polypeptide for a period sufficient to form the complex, and detecting the complex, so that if a complex is detected, a polypeptide of the invention is detected. VEGF-C protein detection can be accomplished using antibodies specific for the protein in any of a number of formats commonly used by those of skill in the art for such detection. In some embodiments, the compound is a polypeptide comprising an ECD fragment of VEGFR-3 that binds VEGF-C.

For example, the production and characterization of monoclonal antibodies specific for VEGF-C is described in U.S. Pat. Nos. 7,109,308; 7,208,582; 7,402,312; 7,423,125; 7,576,189 and 7,850,963, the disclosures of which are incorporated herein by reference in their entireties. Such antibodies may be employed in ELISA-based techniques and Western blotting techniques to detect the presence of VEGF-C in a biological sample from a subject being tested. Methods for setting up ELISA assays and preparing Western blots of a sample are well known to those of skill in the art. The biological sample can be any tissue or fluid in which cancer cells or tissue might be present.

In one embodiment, the antibodies or fragments can be utilized in enzyme immunoassays, wherein the subject antibody or fragment, or second antibodies, are conjugated to an enzyme. When a biological sample comprising a VEGF-C protein is combined with the subject antibodies, binding occurs between the antibodies and the VEGF-C protein. In one embodiment, a biological sample containing cells expressing a mammalian VEGF-C protein, or biological fluid containing secreted VEGF-C is combined with the subject antibodies, and binding occurs between the antibodies and the VEGF-C protein present in the biological sample comprising an epitope recognized by the antibody. This bound protein can be separated from unbound reagents and the presence of the antibody-enzyme conjugate specifically bound to the VEGF-C protein can be determined, for example, by contacting the sample with a substrate of the enzyme which produces a color or other detectable change when acted on by the enzyme. In another embodiment, the subject antibodies can be unlabeled, and a second, labeled antibody can be added which recognizes the subject antibody.

Immunohistochemistry

Assaying VEGF-C by IHC requires at least one anti-VEGF-C antibody. Using standard approaches the anti-VEGF-C antibody can be used to detect the presence of VEGF-C protein in sections obtained from tumors, including paraffin-embedded and frozen tumor sections. Typically, the tumor sections are initially treated in such a way as to retrieve the antigenic structure of proteins that were fixed in the initial process of collecting and preserving the tumor material. Slides are then blocked to prevent non-specific binding by the anti-VEGF-C detection antibody using a blocking reagent such as bovine serum albumin (BSA) or non-fat dried milk. The presence of VEGF-C protein is then detected by binding of the anti-VEGF-C antibody to the VEGF-C protein. The detection antibody is linked to an enzyme, either directly or indirectly, e.g., through a secondary antibody that specifically recognizes the detection antibody. Typically, the tumor sections are washed between steps. The slide is developed using an appropriate enzyme substrate to produce a visible signal, and the samples are then counterstained with hematoxylin.

Similarly, the present invention also relates to a method of detecting and/or quantitating expression of a mammalian VEGF-C protein or a portion of the VEGF-C protein by a cell, in which a composition comprising a cell or fraction thereof (e.g., a soluble fraction) is contacted with an antibody or functional fragment thereof which binds to a mammalian VEGF-C protein or a portion of the VEGF-C protein under conditions appropriate for binding of the antibody or fragment thereto, and binding is monitored. Detection of the antibody, indicative of the formation of a complex between antibody and or a portion of the protein, indicates the presence of the protein.

The method can be used to detect expression of VEGF-C in a biological sample of a mammalian subject (e.g., in a sample, such as a body fluid, such as blood, saliva or other suitable sample). The level of expression of in a biological sample of that subject can also be determined, for instance, by flow cytometry, and the level of expression (e.g., staining intensity) can be correlated with disease susceptibility, progression or risk.

In certain other embodiments, the polynucleotides such as mRNA encoding a mammalian VEGF-C protein may be used for the identification of subjects that would benefit from treatment with a Notch inhibitor. In general, methods for detecting VEGF-C mRNA can comprise contacting a biological sample with a compound that binds to and forms a complex with VEGF-C mRNA for a period sufficient to form the complex, and detecting the complex in a quantitative or semi-quantitative way. Such methods can also comprise amplification techniques involving contacting a biological sample with nucleic acid primers that anneal to VEGF-C mRNA or its complement, and amplifying annealed polynucleotides, so that if a polynucleotide is amplified, a VEGF-C polynucleotide is detected.

In the amplification procedures, polynucleotide sequences encoding a VEGF-C protein may be used in hybridization or PCR assays of fluids or tissues from biopsies to detect VEGF-C protein expression. Such methods may be qualitative or quantitative in nature and may include Southern or northern analysis, dot blot or other membrane-based technologies; PCR technologies; dip stick, pin, chip and ELISA technologies. All of these techniques are well known in the art and are the basis of many commercially available diagnostic kits.

One such procedure known in the art is quantitative real-time PCR. Real-time quantitative PCR can be conveniently accomplished using the commercially available ABI PRISM® 7700 Sequence Detection System, available from PE-Applied Biosystems, Foster City, Calif. and used according to manufacturer's instructions. PCR reagents can be obtained from PE-Applied Biosystems, Foster City, Calif. Gene target quantities obtained by real time RT-PCR may be normalized using either the expression level of GAPDH, a gene whose expression is constant, or by quantifying total RNA using RiboGreenT™ (Molecular Probes, Inc. Eugene, Oreg.). GAPDH expression is quantified by real time RT-PCR, by being run simultaneously with the target, multiplexing, or separately. Total RNA is quantified using RiboGreen™ RNA quantification reagent from Molecular Probes. Methods of RNA quantification by RiboGreen™ are taught in Jones et al. (1998). Controls are analyzed in parallel to verify the absence of DNA in the RNA preparation (-RT control) as well as the absence of primer dimers in control samples lacking template RNA. In addition, RT-PCR products may be analyzed by gel electrophoresis.

A reverse transcriptase PCR(RT-PCR) amplification procedure may be performed in order to quantify the amount of mRNA amplified. Methods of reverse transcribing RNA into cDNA are well known and described in Sambrook et al. (1989). Alternative methods for reverse transcription utilize thermostable DNA polymerases. These methods are described in WO 90/07641, filed Dec. 21, 1990.

Conditions for incubating a nucleic acid probe or antibody with a test sample vary. Incubation conditions depend on the format employed in the assay, the detection methods employed, and the type and nature of the nucleic acid probe or antibody used in the assay. One skilled in the art will recognize that any one of the commonly available hybridization, amplification or immunological assay formats can readily be adapted to employ the nucleic acid probes or antibodies of the present invention. Examples of such assays can be found in Chard (1986); Bullock et al., (1982, 1983 and 1985); and Tijssen (1985). The tests of the present invention include cells, protein extracts of cells, or biological fluids such as, blood, serum, and plasma. The test sample used in the above-described method will vary based on the assay format, nature of the detection method and the tissues, cells or extracts used as the sample to be assayed. Methods for preparing protein extracts or membrane extracts of cells are well known in the art and can be readily be adapted in order to obtain a sample which is compatible with the system utilized.

In addition, such assays may be useful in evaluating the efficacy of a particular therapeutic treatment regime in animal studies, in clinical trials, or in monitoring the treatment of an individual patient.

In some variations, to provide a basis for the identification of patients for treatment with a Notch inhibitor, VEGF-C measurements from multiple individuals are obtained, both in healthy tissues and cancer tissue, to establish a data set of VEGF-C mRNA or protein expression. With an established data set a variety of standard statistical analyses can be performed to identify when a measurement of VEGF-C is elevated, e.g., in a statistically significant manner relative to a healthy control. To assess the relative level of VEGF-C expression, the level of VEGF-C expression in a cancer tissue sample can be subjected to one or more of various comparisons. In general, it can be compared to: (a) VEGF-C mRNA or protein expression level(s) in normal tissue from the organ in which the cancer originated; (b) VEGF-C mRNA or protein expression levels in a collection of comparable cancer tissue samples; (c) VEGF-C mRNA or protein expression level in a collection of normal tissue samples; or (d) VEGF-C mRNA or protein expression level in an arbitrary standard. In some embodiments, a VEGF-C measurement of at least 1.0, 1.5, 2.0, 2.5 or at least 3.0 standard deviation(s) greater than a median VEGF-C measurement in corresponding healthy tissue is scored as elevated VEGF-C expression. In other embodiments, a VEGF-C measurement that is statistically significantly greater than VEGF-C measurements in corresponding healthy tissue, with a p-value less than 0.1, or less than 0.05, or less than 0.01, or less than 0.005, or less than 0.001 is scored as elevated VEGF-C expression. As a data set enlarges, the comparison can be refined by stratifying the data for additional variables, such as the age, sex, ethnicity, body mass, smoking habits or other factors that differentiate subjects.

In some embodiments, the screening methods described herein comprise comparing VEGF-C expression in the cancer tissue sample with VEGF-C expression in healthy tissue of the same type as the tumor, wherein elevated VEGF-C expression in the tumor compared to the healthy tissue identifies the subject as a subject for whom Notch-targeted therapy will have efficacy.

In some embodiments, the screening methods described herein further comprise measuring the expression of at least one gene selected from the group consisting of HES1, HES4, HES5, HESL, HEY-2, DTX1, MYC, NRARP, PTCRA, SHQ1, and HeyL (hairy/enhancer-of-split related with YRPW motif-like) in the biological sample, wherein elevated VEGF-C expression and elevated expression of the second gene in the biological sample correlate with a pathological phenotype (i.e., cancer). See International Publication Nos. WO 2010/005644 and WO 2009/032084, the disclosures of which are incorporated herein by reference in their entireties. It is contemplated that subjects with elevated VEGF-C and elevated expression of one or more of these additional markers will be especially good candidates for Notch-targeted therapy.

The screening methods described herein may optionally comprise the step of prescribing for or administering to the subject identified as having elevated VEGF-C expression in the biological sample a composition comprising a molecule that suppresses expression of downstream activity of Notch (i.e., a Notch inhibitor). Br “prescribing” is meant provising an order or authorization for the therapy, which may be dispensed to the subject for self-administration and/or administered by a medical professional that is difference from the prescribing professional.

Therapeutic Methods

Also described herein are methods of treatment comprising obtaining a tumor or tumor biopsy from a mammalian subject, determining that the tumor or tumor biopsy has elevated expression of VEGF-C, and prescribing for or administering to the subject a composition comprising a molecule that suppresses expression or signaling activity of Notch. The term “Notch” as used herein collectively refers to any one of the Notch1, Notch2, Notch 3 and Notch4 receptors. Compositions that inhibit at least Notch4 and Notch1 signaling are preferred in some embodiments.

Inhibition of Notch signaling can be achieved at many different levels. Agents targeted to some of these levels have been described (Rizzo et al., Oncogene, 27:5124-5131, 2008, FIG. 1 in particular). For example, it is possible to interfere with Notch-ligand interactions by using receptor decoys (Nickoloff et al., 2002), blocking ligand ubiquitination/trans-endocytosis (Pitsouli et al., 2005), or Notch receptor fucosylation (Okajima et al., 2002). It is also possible to interfere with receptor activation by blocking ligand-induced conformation changes in Notch receptors (Gordon et al., 2007), receptor cleavage by ADAM proteases (Brou et al., 2000) or γ-secretase (Kopan et al., 2004; Miele et al., 2006) as well as Notch mono-ubiquitination (Gupta-Rossi et al., 2004).

Inhibition of Notch signaling could also be achieved by disrupting protein-protein interactions involved in Notch-dependent nuclear events (Nam et al., 2007; Nam et al., 2006), including assembly of co-activators with the Notch transcriptional complex (NTC) and formation of higher order DNA-bound complexes.

Gamma-secretase inhibitors are currently undergoing clinical trials and antibodies that “lock” Notch receptors in an inactive conformation by binding to the negative regulatory region are in preclinical development (Li et al., 2008). Monoclonal antibodies that target the Notch ligand Dll4 (Ridgway et al., 2006) have been shown to inhibit Notch signaling in endothelial cells and cause disorganized angiogenesis.

In some embodiments, molecules that suppress expression or signaling activity of Notch (i.e., a Notch inhibitor) include, but are not limited to an antibody that binds a Notch protein and inhibits ligand-mediated stimulation of Notch signaling; a soluble polypeptide that comprises an ECD fragment of a Notch polypeptide that binds a Notch ligand and inhibits the ligand from stimulation of Notch signaling; an antisense or interfering nucleic acid (e.g., antisense oligonucleotide; micro-RNA, short interfering RNA) that inhibits Notch expression; an antibody that binds a Notch ligand protein and inhibits the ligand from stimulation of Notch signaling; a soluble notch ligand polypeptide that comprises an ECD fragment of a Notch ligand that binds Notch and inhibits stimulation of Notch by ligand expressed by a cell; an antisense or interfering nucleic acid (e.g., antisense oligonucleotide; micro-RNA, short interfering RNA) that inhibits expression of a Notch ligand; a small molecule that inhibits Notch expression or signaling; a molecule that inhibits proteolytic cleavage-activation of Notch; and a molecule that inhibits Notch NICD peptide from binding core binding factor-1 (CBF-1) or activating transcription of one or more genes selected from HES, Myc, and p21.

In some embodiments, the Notch ligand is delta-like ligand 4 (Dll4) and the Notch inhibitor is an antibody that binds a Notch protein and inhibits delta-like ligand 4 (Dll4) stimulation of Notch signaling; a soluble polypeptide that comprises an ECD fragment of a Notch polypeptide that binds Dll4 and inhibits Dll4 stimulation of Notch signaling; an antisense or interfering nucleic acid (e.g., antisense oligonucleotide; micro-RNA, short interfering RNA) that inhibits Notch expression; an antibody that binds a Dll4 protein and inhibits Dll4 stimulation of Notch signaling; a soluble Dll4 polypeptide that comprises an ECD fragment of Dll4 that binds Notch and inhibits stimulation of Notch by cellular Dll4; or an antisense or interfering nucleic acid (e.g., antisense oligonucleotide; micro-RNA, short interfering RNA) that inhibits Dll4 expression.

Various agents that inhibit Notch receptor activation are known. For example, small molecule inhibitors of the TNFα converting enzymes (TACE inhibitors), including ADAM10 and ADAM17 (Moss et al, 2008), and γ-secretase inhibitors (DeStrooper et al, 1999) that inhibit Notch receptor activation by inhibiting proteolytic cleavage of the Notch receptor. Soluble receptor decoys that sequester Notch ligands can be used to inhibit Notch receptor activation (Funahashi et al, 2008). Also, soluble ligands that inhibit ligand binding to Notch receptors (Noguera-Troise et al, 2006) can be used. Antibodies that bind to Notch ligands (Ridgway et al, 2006; Noguera-Troise et al, 2006) or to Notch receptors (Li et al, 2008) can be used to inhibit Notch receptor activation. In addition, antibodies that bind to components of the γ-secretase complex, e.g. nicastrin, can be used.

A. Antibodies

In some embodiments, the Notch inhibitor is an antibody (e.g., an antibody that binds a Notch protein or a Notch ligand protein and inhibits Notch ligand induced (e.g., Dll4-induced) stimulation of Notch signaling). Such antibodies include, but are not limited to, polyclonal, monoclonal, chimeric, single chain, Fab fragments and fragments produced by a Fab expression library, bifunctional/bispecific antibodies, humanized antibodies, CDR grafted antibodies, human antibodies and antibodies which include portions of CDR sequences specific for a Notch protein or a Notch ligand protein.

Neutralizing antibodies, i.e., those which may inhibit Notch ligand induced (e.g., Dll4-induced) stimulation of Notch signaling, are especially preferred. In a preferred embodiment, the antibody is a monoclonal antibody. In some embodiments, the antibody is a human or a humanized antibody. Antibodies that bind to ECD epitopes of a Notch protein or Notch ligand protein are preferred.

Techniques described below are useful for the preparation of antibodies for both detection and therapeutic purposes. Means for preparing and characterizing antibodies are well known in the art (see, e.g., Harlow and Lane, 1988). Briefly, a polyclonal antibody is prepared by immunizing an animal with an immunogen comprising a polypeptide of the present invention and collecting antisera from that immunized animal. A wide range of animal species can be used for the production of antisera. Typically an animal used for production of anti-antisera is a non-human animal including rabbits, mice, rats, hamsters, goat, sheep, pigs or horses. Because of the relatively large blood volume of rabbits, a rabbit is a preferred choice for production of polyclonal antibodies.

Antibodies, both polyclonal and monoclonal, specific for isoforms of antigen may be prepared using conventional immunization techniques, as will be generally known to those of skill in the art. As used herein, the term “specific for” is intended to mean that the variable regions of the antibodies recognize and bind a Notch protein or a Notch ligand protein and are capable of distinguishing a Notch protein or a Notch ligand protein from other antigens. A composition containing antigenic epitopes of a Notch protein or a Notch ligand protein can be used to immunize one or more experimental animals, such as a rabbit or mouse, which will then proceed to produce specific antibodies against the Notch protein or a Notch ligand protein. Polyclonal antisera may be obtained, after allowing time for antibody generation, simply by bleeding the animal and preparing serum samples from the whole blood.

Monoclonal antibodies to a Notch protein or a Notch ligand protein may be prepared using any technique which provides for the production of antibody molecules by continuous cell lines in culture. These include but are not limited to the hybridoma technique originally described by Koehler and Milstein (1975), the human B-cell hybridoma technique (Kosbor et al., 1983; Cote et al., 1983) and the EBV-hybridoma technique (Cole et al., 1985).

Methods of making antibody fusion proteins are well known in the art. See, e.g., U.S. Pat. No. 6,306,393, the disclosure of which is incorporated herein by reference in its entirety. In certain embodiments of the invention, fusion proteins are produced which may include a flexible linker, which connects the chimeric scFv antibody to the heterologous protein moiety. Appropriate linker sequences are those that do not affect the ability of the resulting fusion protein to be recognized and bind the epitope specifically bound by the V domain of the protein (see, e.g., WO 98/25965, the disclosure of which is incorporated herein by reference in its entirety).

In addition to the production of monoclonal antibodies, techniques developed for the production of “chimeric antibodies”, the splicing of mouse antibody genes to human antibody genes to obtain a molecule with appropriate antigen specificity and biological activity can be used (Morrison et al., 1984; Neuberger et al., 1984; Takeda et al., 1985). Alternatively, techniques described for the production of single chain antibodies (U.S. Pat. No. 4,946,778) can be adapted to produce a Notch protein or a Notch ligand protein-specific single chain antibodies.

Antibodies may also be produced by inducing in vivo production in the lymphocyte population or by screening recombinant immunoglobulin libraries or panels of highly specific binding reagents as disclosed in Orlandi et al. (1989), and Winter and Milstein (1991).

Fully human antibodies relate to antibody molecules in which essentially the entire sequences of both the light chain and the heavy chain, including the CDRs, arise from human genes. Such antibodies are termed “human antibodies,” or “fully human antibodies” herein. Human monoclonal antibodies can be prepared by the trioma technique; the human B cell hybridoma technique (see Kozbor et al., 1983) and the EBV hybridoma technique to produce human monoclonal antibodies (see Cole et al., 1985). Human monoclonal antibodies may be utilized in the practice of the present invention and may be produced by using human hybridomas (see Cote et al., 1983) or by transforming human B cells with Epstein Barr Virus in vitro (see Cole et al., 1985).

In addition, human antibodies can also be produced using additional techniques, including phage display libraries (Hoogenboom and Winter 1992; Marks et al., 1991). Similarly, human antibodies can be made by introducing human immunoglobulin loci into transgenic animals, e.g., mice in which the endogenous immunoglobulin genes have been partially or completely inactivated. Upon challenge, human antibody production is observed, which closely resembles that seen in humans in all respects, including gene rearrangement, assembly, and antibody repertoire. This approach is described, for example, in U.S. Pat. Nos. 5,545,807; 5,545,806; 5,569,825; 5,625,126; 5,633,425; 5,661,016, and in Marks et al. (1992); Lonberg et al. (1994); Morrison (1994); Fishwild et al., (1996); Neuberger (1996); and Lonberg and Huszar (1995).

Human antibodies may additionally be produced using transgenic nonhuman animals which are modified so as to produce fully human antibodies rather than the animal's endogenous antibodies in response to challenge by an antigen. (See PCT publication WO94/02602). The endogenous genes encoding the heavy and light immunoglobulin chains in the nonhuman host have been incapacitated, and active loci encoding human heavy and light chain immunoglobulins are inserted into the host's genome. The human genes are incorporated, for example, using yeast artificial chromosomes containing the requisite human DNA segments. An animal which provides all the desired modifications is then obtained as progeny by crossbreeding intermediate transgenic animals containing fewer than the full complement of the modifications. The preferred embodiment of such a nonhuman animal is a mouse, and is termed the Xenomouse™ as disclosed in PCT publications WO 96/33735 and WO 96/34096. This animal produces B cells which secrete fully human immunoglobulins. The antibodies can be obtained directly from the animal after immunization with an immunogen of interest, as, for example, a preparation of a polyclonal antibody, or alternatively from immortalized B cells derived from the animal, such as hybridomas producing monoclonal antibodies. Additionally, the genes encoding the immunoglobulins with human variable regions can be recovered and expressed to obtain the antibodies directly, or can be further modified to obtain analogs of antibodies such as, for example, single chain Fv molecules.

An example of a method of producing a nonhuman host, exemplified as a mouse, lacking expression of an endogenous immunoglobulin heavy chain is disclosed in U.S. Pat. No. 5,939,598. It can be obtained by a method including deleting the J segment genes from at least one endogenous heavy chain locus in an embryonic stem cell to prevent rearrangement of the locus and to prevent formation of a transcript of a rearranged immunoglobulin heavy chain locus, the deletion being effected by a targeting vector containing a gene encoding a selectable marker; and producing from the embryonic stem cell a transgenic mouse whose somatic and germ cells contain the gene encoding the selectable marker.

B. Antisense Inhibitors

In some embodiments, the Notch inhibitor is an antisense or interfering nucleic acid that inhibits Notch or a Notch ligand protein (e.g., Dll4) expression. Polynucleotide products which are useful in this endeavor include, but are not limited to, antisense oligonucleotides, ribozymes, small interfering RNAs, natural or designed microRNAs and triple helix polynucleotides.

Techniques for making and delivering antisense polynucleotides and ribozymes are well known to those in the art and have been extensively described in scientific, patent, and trade literature. (PCT Publication No. WO 00/32765; Lima et al., 1997; Kurreck et al., 2002; Crooke and Lebleu, 1993; Melton, 1988). Anti-sense RNA and DNA molecules act to directly block the translation of mRNA by binding to targeted mRNA and preventing protein translation. An example of an antisense polynucleotide is an oligodeoxyribonucleotide derived from the translation initiation site, e.g., between −10 and +10 regions of the relevant nucleotide sequence. Antisense oligonucleotides of 8-200 nucleotides in length that include at least a portion of this region of the Notch or a Notch ligand protein (e.g., Dll4) cDNA or genomic sequences set forth in Table 1 below (or are complementary to) are preferred antisense inhibitors of the invention.

TABLE 1

| cDNA | Swiss-Prot Accession No. | SEQ ID NO: |
|---|---|---|
| Notch-1 | P46531 | 3 |
| Notch-2 | Q04721 | 5 |
| Notch-3 | Q9UM47 | 7 |
| Notch-4 | Q99466 | 9 |
| Dll4 | Q9NR61 | 15 |
| Jagged1 | P78504 | 17 |
| Jagged2 | Q9Y219 | 19 |
| Dll1 | O00548 | 11 |
| Dll3 | Q9NYJ7 | 13 |

Antisense polynucleotides are typically generated within the cell by expression from antisense constructs that contain the antisense nucleic acid strand as the transcribed strand. Antisense methodology takes advantage of the fact that nucleic acids tend to pair with “complementary” sequences. By complementary, it is meant that polynucleotides are those which are capable of base-pairing according to the standard Watson-Crick complementarity rules. That is, the larger purines will base pair with the smaller pyrimidines to form combinations of guanine paired with cytosine (G:C) and adenine paired with either thymine (A:T) in the case of DNA, or adenine paired with uracil (A:U) in the case of RNA. Inclusion of less common bases such as inosine, 5-methylcytosine, 6-methyladenine, hypoxanthine and others in hybridizing sequences does not interfere with pairing.

Targeting double-stranded (ds) DNA with polynucleotides leads to triple-helix formation; targeting RNA will lead to double-helix formation. Antisense polynucleotides, when introduced into a target cell, specifically bind to their target polynucleotide and interfere with transcription, RNA processing, transport, translation and/or stability. Antisense RNA constructs, or DNA encoding such antisense RNAs, may be employed to inhibit gene transcription or translation or both within a host cell, either in vitro or in vivo, such as within a host animal, including a human subject.

Antisense constructs may be designed to bind to the promoter and other control regions, exons, introns or even exon-intron boundaries of a gene. Highly effective antisense constructs include regions complementary to intron/exon splice junctions. Thus, a preferred embodiment includes an antisense construct with complementarity to regions within 50-200 bases of an intron-exon splice junction. It has been observed that some exon sequences can be included in the construct without seriously affecting the target selectivity thereof. The amount of exonic material included will vary depending on the particular exon and intron sequences used. One can readily test whether too much exon DNA is included simply by testing the constructs in vitro to determine whether normal cellular function is affected or whether the expression of related genes having complementary sequences is affected.

For purposes of making antisense oligonucleotides, polynucleotide sequences that are substantially complementary over their entire length and have zero or very few base mismatches are preferred. For example, sequences of fifteen bases in length preferably have complementary nucleotides at thirteen or fourteen or fifteen positions. Naturally, sequences which are completely complementary will be sequences which are entirely complementary throughout their entire length and have no base mismatches. Other sequences with lower degrees of homology also are contemplated. For example, an antisense construct which has limited regions of high homology, but also contains a non-homologous region (e.g., ribozymes) could be designed. These molecules, though having less than 50% homology, would bind to target sequences under appropriate conditions.

Methods for designing and optimizing antisense nucleotides are described in Lima et al. (1997) and Kurreck et al. (2002). Additionally, commercial software and online resources are available to optimize antisense sequence selection and also to compare selected sequences to known genomic sequences to help ensure uniqueness/specificity for a chosen gene. (See, e.g., world wide web at sfold.wadsworth.org/index.pl.) Such uniqueness can be further confirmed by hybridization analyses. Antisense nucleic acids are introduced into cells (e.g., by a viral vector or colloidal dispersion system such as a liposome).

Although antisense sequences may be full length genomic or cDNA copies, they also may be shorter fragments or oligonucleotides e.g., polynucleotides of 100 or less bases. Although shorter oligomers (8-20) are easier to make and more easily permeable in vivo, other factors also are involved in determining the specificity of base pairing. For example, the binding affinity and sequence specificity of an oligonucleotide to its complementary target increases with increasing length. It is contemplated that oligonucleotides of 8, 9, 10, 11, 12, 13, 14, 15, 16, 17, 18, 19, 20, 25, 30, 35, 40, 45, 50, or more base pairs will be used.

Ribozymes are enzymatic RNA molecules capable of catalyzing the specific cleavage of RNA. The cleavage event renders the mRNA unstable and prevents protein expression. The mechanism of ribozyme action involves sequence specific interaction of the ribozyme molecule to complementary target RNA, followed by an endonucleolytic cleavage. Within the scope of the invention are engineered hammerhead, for which the substrate sequence requirements are minimal, or other motif ribozyme molecules that specifically and efficiently catalyze endonucleolytic cleavage of RNA sequences encoding protein complex components. Design of the hammerhead ribozyme and the therapeutic uses of ribozymes are disclosed in Usman et al. (1996). Ribozymes can also be prepared and used as described in Long et al. (1993); Symons (1992); Perrotta et al. (1992); Ojwang et al. (1992); and U.S. Pat. No. 5,254,678. Methods of cleaving RNA using ribozymes is described in U.S. Pat. No. 5,116,742; and methods for increasing the specificity of ribozymes are described in U.S. Pat. No. 5,225,337 and Koizumi et al. (1989). Preparation and use of ribozyme fragments in a hairpin structure are described by Chowrira and Burke (1992). Ribozymes can also be made by rolling transcription (Daubendiek and Kool, 1997).

The full-length gene need not be known in order to design and use specific inhibitory ribozymes. Specific ribozyme cleavage sites within any potential RNA target are initially identified by scanning the target molecule for ribozyme cleavage sites which include the following sequences, GUA, GUU, and GUC. Once identified, short RNA sequences of between 15 and 20 ribonucleotides corresponding to the region of the target gene containing the cleavage site may be evaluated for predicted structural features, such as secondary structure, that may render the oligonucleotide sequence unsuitable. The suitability of candidate targets may also be evaluated by testing their accessibility to hybridization with complementary oligonucleotides, using ribonuclease protection assays (Draper PCT WO 93/23569; and U.S. Pat. No. 5,093,246, incorporated herein by reference). Using the nucleic acid sequences disclosed herein and methods known in the art, ribozymes can be designed to specifically bind and cut the corresponding mRNA species. Ribozymes, therefore, provide a means to inhibit the expression Notch (or Dll4).

Alternatively, endogenous gene expression can be reduced by inactivating or “knocking out” the gene or its promoter using targeted homologous recombination. (e.g., see Smithies et al., 1985; Thomas and Capecchi, 1987; Thompson et al., 1989). For example, a mutant, non-functional gene (or a completely unrelated DNA sequence) flanked by DNA homologous to the endogenous gene (either the coding regions or regulatory regions of the gene) can be used to transfect cells that express that gene in vivo. Insertion of the DNA construct, via targeted homologous recombination, results in inactivation of the gene.

Gene expression can also be reduced by targeting deoxyribonucleotide sequences complementary to the regulatory region of the target gene (i.e., the gene promoter and/or enhancers) to form triple helical structures that prevent transcription of the gene in target cells in the body. (See generally, Helene, 1991; Helene et al., 1992; and Maher, 1992). Nucleic acid molecules used in triple helix formation for the inhibition of transcription are generally single stranded deoxyribonucleotides. The base composition must be designed to promote triple helix formation via Hoogsteen base pairing rules, which generally require sizeable stretches of either purines or pyrimidines to be present on one strand of a duplex. Nucleotide sequences may be pyrimidine-based, which will result in TAT and CGC+triplets across the three associated strands of the resulting triple helix. The pyrimidine-rich molecules provide base complementarity to a purine-rich region of a single strand of the duplex in a parallel orientation to that strand. In addition, nucleic acid molecules may be chosen that are purine-rich, for example, containing a stretch of G residues. These molecules will form a triple helix with a DNA duplex that is rich in GC pairs, in which the majority of the purine residues are located on a single strand of the targeted duplex, resulting in GGC triplets across the three strands in the triplex.

Alternatively, the potential sequences that can be targeted for triple helix formation may be increased by creating a so called “switchback” nucleic acid molecule. Switchback molecules are synthesized in an alternating 5′-3′,3′-5′ manner, such that they base pair with first one strand of a duplex and then the other, eliminating the necessity for a sizeable stretch of either purines or pyrimidines to be present on one strand of a duplex.

Another technique for inhibiting the expression of a gene involves the use of RNA for induction of RNA interference (RNAi), using double stranded (dsRNA) (Fire et al., 1998) or small interfering RNA (siRNA) sequences (Elbashir et al., 2001); Yu et al., 2002. “RNAi” is the process by which dsRNA induces homology-dependent degradation of complimentary mRNA. The presence of dsRNA in cells triggers the RNAi response though a mechanism that has yet to be fully characterized. In one embodiment, a synthetic antisense nucleic acid molecule is hybridized by complementary base pairing with a “sense” ribonucleic acid to form a double stranded RNA. The presence of long dsRNAs in cells stimulates the activity of a ribonuclease III enzyme. The dsRNA antisense and sense nucleic acid molecules are provided that correspond to at least about 20, 25, 50, 100, 250 or 500 nucleotides or an entire Notch (or a Notch ligand protein) coding strand, or to only a portion thereof. In an alternative embodiment, the siRNAs are 30 nucleotides or less in length, and more preferably 21- to 23-nucleotides, with characteristic 2- to 3-nucleotide 3′-overhanging ends, which are generated by ribonuclease III cleavage from longer dsRNAs. (See e.g. Tuschl, 2002). At notably higher concentrations single stranded 21 nucleotide RNA molecules have been also shown to function as siRNAs (i.e., enter the RNAi pathway and specifically target mRNA for degradation in mammalian cells (Martinez et al., 2002). Cleavage of the target RNA takes place in the middle of the region complementary to the antisense strand of the siRNA duplex (Elbashir et al., 2001).

Intracellular transcription of small RNA molecules can be achieved by cloning the siRNA templates into RNA polymerase III (Pol III) transcription units, which normally encode the small nuclear RNA (snRNA) U6 or the human RNAse P RNA H1. Two approaches can be used to express siRNAs: in one embodiment, sense and antisense strands constituting the siRNA duplex are transcribed using constructs with individual promoters (Lee et al., 2002); in an alternative embodiment, siRNAs are expressed as stem-loop hairpin RNA structures that give rise to siRNAs after intracellular processing (Brummelkamp et al., 2002, herein incorporated by reference). Alternatively, a stem loop hairpin can be expressed within an unrelated Pol II transcribed mRNA transcript. A stem-loop hairpin designed to contain the siRNA sequence also contains conserved microRNA sequences within the loop and stem regions, thus resembling a natural precursor mRNA structure. Subsequently, the precursor can be processed by the cellular RNAi components to yield mature, functional siRNA/miRNA. (See, generally, Zeng et al., 2002; Hutvagner et al., 2002; Kawasake et al., 2003).

RNAi has been studied in a variety of systems. Work in Drosophila embryonic lysates (Elbashir et al., 2001) has revealed certain requirements for siRNA length, structure, chemical composition, and sequence that are essential to mediate efficient RNAi activity. Twenty-one nucleotide siRNA duplexes are most active when containing two nucleotide 3′-overhangs. Replacing the 3′-overhanging segments of a 21-mer siRNA duplex having 2 nucleotide 3′ overhangs with deoxyribonucleotides has no adverse effect on RNAi activity, while, replacing up to 4 nucleotides on each end of the siRNA with deoxyribonucleotides may be well tolerated. Complete substitution with deoxyribonucleotides results in no RNAi activity (Elbashir et al., 2001).

Furthermore, complete substitution of one or both siRNA strands with 2′-deoxy (2′-H) or 2′-O-methyl nucleotides results in no RNAi activity, whereas substitution of the 3′-terminal siRNA overhang nucleotides with deoxy nucleotides (2′-H) is tolerated. Single mismatch sequences in the center of the siRNA duplex may abolish RNAi activity. In addition, studies indicate that the position of the cleavage site in the target RNA is defined by the 5′-end of the siRNA guide sequence rather than the 3′-end (Elbashir et al., 2001). Other studies indicate that a 5′-phosphate on the target-complementary strand of a siRNA duplex is required for siRNA activity and that ATP is utilized to maintain the 5′-phosphate moiety on the siRNA (Nykanen et al., 2001).

The dsRNA/siRNA is most commonly administered by annealing sense and antisense RNA strands in vitro before delivery to the organism. In an alternate embodiment, RNAi may be carried out by administering sense and antisense nucleic acids of the invention in the same solution without annealing prior to administration, and may even be performed by administering the nucleic acids in separate vehicles within a very close timeframe.

Genetic control can also be achieved through the design of novel transcription factors for modulating expression of the gene of interest in native cells and animals. For example, the Cys2-His2 zinc finger proteins, which bind DNA via their zinc finger domains, have been shown to be amenable to structural changes that lead to the recognition of different target sequences. These artificial zinc finger proteins recognize specific target sites with high affinity and low dissociation constants, and are able to act as gene switches to modulate gene expression. Knowledge of the particular target sequence of the present invention facilitates the engineering of zinc finger proteins specific for the target sequence using known methods such as a combination of structure-based modeling and screening of phage display libraries (Segal et al. 1999; Liu et al., 1997; Greisman and Pabo, 1997; Choo et al., 1997). Each zinc finger domain usually recognizes three or more base pairs. Since a recognition sequence of 18 base pairs is generally sufficient in length to render it unique in any known genome, a zinc finger protein consisting of 6 tandem repeats of zinc fingers would be expected to ensure specificity for a particular sequence (Segal et al., 1999). The artificial zinc finger repeats, designed based on target sequences, are fused to activation or repression domains to promote or suppress gene expression (Liu et al., 1997). Alternatively, the zinc finger domains can be fused to the TATA box-binding factor (TBP) with varying lengths of linker region between the zinc finger peptide and the TBP to create either transcriptional activators or repressors (Kim et al., 1997). Such proteins, and polynucleotides that encode them, have utility for modulating expression in vivo in both native cells, animals and humans. The novel transcription factor can be delivered to the target cells by transfecting constructs that express the transcription factor (gene therapy), or by introducing the protein. Engineered zinc finger proteins can also be designed to bind RNA sequences for use in therapeutics as alternatives to antisense or catalytic RNA methods (McColl et al., 1999; Wu et al., 1995).

Anti-sense RNA and DNA molecules, ribozymes, RNAi, triple helix polynucleotides, and novel transcription factors can be prepared by any method known in the art for the synthesis of DNA and RNA molecules. These include techniques for chemically synthesizing oligodeoxyribonucleotides well known in the art including, but not limited to, solid phase phosphoramidite chemical synthesis. Alternatively, RNA molecules may be generated by in vitro and in vivo transcription of DNA sequences encoding the antisense RNA molecule. Such DNA sequences may be incorporated into a wide variety of vectors which incorporate suitable RNA polymerase promoters such as the T7 or SP6 polymerase promoters. Alternatively, antisense cDNA constructs that synthesize antisense RNA constitutively or inducibly, depending on the promoter used, can be introduced stably or transiently into cells.

C. Soluble Polypeptide Inhibitors

In some embodiments, the Notch inhibitor is a soluble polypeptide that comprises an ECD fragment of a Notch polypeptide that binds Dll4 (or another Notch ligand) and inhibits Dll4 (or another Notch ligand) stimulation of Notch signaling. In other embodiments, the Notch inhibitor is a soluble Dll4 polypeptide that comprises an ECD fragment of Dll4 (or another Notch ligand) that binds Notch and inhibits stimulation of Notch by cellular Dll4. Fusion proteins comprising a soluble polypeptide inhibitor, and a heterologous polypeptide, are also contemplated. Nonlimiting examples of heterologous polypeptides which can be fused to polypeptides of interest include proteins with long circulating half-life, such as, but not limited to, immunoglobulin constant regions (e.g., Fc region); marker sequences that permit identification of the polypeptide of interest; sequences that facilitate purification of the polypeptide of interest; and sequences that promote formation of multimeric proteins. In some embodiments, a receptor fragment is fused to alkaline phosphatase (AP). Methods for making Fc or AP fusion constructs are found in WO 02/060950.

D. Other Notch Inhibitors

Other Notch inhibitors contemplated for use in the treatment methods described herein include the following agents listed below in Table 1A.

TABLE 1A

| Inhibitor Name | Chemical name/Structure | Mechanism of Action |
|---|---|---|
| γ-secretase inhibitor I | Z-Leu-Leu-Norleucine-CHO | Inhibition of Notch cleavage by γ- |
| (GSI I) | | secretase |
| γ-secretase inhibitor II | | Inhibition of Notch cleavage by γ- |
| (GSI II) | | secretase |
| γ-secretase inhibitor III | N-Benzyloxycarbonyl-Leu- | Inhibition of Notch cleavage by γ- |
| (GSI III) | leucinal | secretase |
| γ-secretase inhibitor III | N-(2-Naphthoyl)-Val- | Inhibition of Notch cleavage by γ- |
| (GSI IV) | phenylalaninal | secretase |
| γ-secretase inhibitor III | N-Benzyloxycarbonyl-Leu- | Inhibition of Notch cleavage by γ- |
| (GSI V) | phenylalaninal | secretase |
| γ-secretase inhibitor III | 1-(S)-endo-N-(1,3,3)- | Inhibition of Notch cleavage by γ- |
| (GSI VI) | Trimethylbicyclo[2.2.1]hept- | secretase |
| | 2-yl)-4-fluorophenyl | |
| | Sulfonamide | |
| γ-secretase inhibitor III | Menthyloxycarbonyl-LL- | Inhibition of Notch cleavage by γ- |
| (GSI VII) | CHO | secretase |
| γ-secretase inhibitor III | (DAPT), N--[N-(3,5- | Inhibition of Notch cleavage by γ- |
| (GSI IX) | Difluorophenacetyl-L- | secretase |
| | alanyl)]-S-phenylglycine t- | |
| | Butyl Ester | |
| γ-secretase inhibitor X | {1S-Benzyl-4R-[1-(1S- | Inhibition of Notch cleavage by γ- |
| (GSI X) | carbamoyl-2- | secretase |
| | phenethylcarbamoyl)-1S-3- | |
| | methylbutylcarb-amoyl]-2R- | |
| | hydroxy-5- | |
| | phenylpentyl}carbamic Acid | |
| | tert-butyl Ester | |
| γ-secretase inhibitor XI | 7-Amino-4-chloro-3- | Inhibition of Notch cleavage by γ- |
| (GSI XI) | methoxyisocoumarin | secretase |
| γ-secretase inhibitor XII | Z-Ile-Leu-CHO | Inhibition of Notch cleavage by γ- |
| (GSI XII) | | secretase |
| γ-secretase inhibitor | Z-Tyr-Ile-Leu-CHO | Inhibition of Notch cleavage by γ- |
| XIII (GSI XIII) | | secretase |
| γ-secretase inhibitor | Z-Cys(t-Bu)-Ile-Leu-CHO | Inhibition of Notch cleavage by γ- |
| XIV (GSI XIV) | | secretase |
| γ-secretase inhibitor | N--[N-3,5- | Inhibition of Notch cleavage by γ- |
| XVI (GSI XVI) | Difluorophenacetyl]-L- | secretase |
| | alanyl-S-phenylglycine | |
| | Methyl Ester | |
| γ-secretase inhibitor | WPE-III-31C | Inhibition of Notch cleavage by γ- |
| XVII (GSI XVII) | | secretase |
| γ-secretase inhibitor | (2S,3R)-3-(3,4- | Inhibition of Notch cleavage by γ- |
| XIX (GSI XIX) | Difluorophenyl)-2-(4- | secretase |
| | fluorophenyl)-4-hydroxy-N- | |
| | ((3S)-2-oxo--5-phenyl-2,3- | |
| | dihydro-1H- | |
| | benzo[e][1,4]diazepin-3-yl)- | |
| | butyramide | |
| γ-secretase inhibitor XX | (Dibenzazepine (DBZ)), | Inhibition of Notch cleavage by γ- |
| (GSI XX) | (S,S)-2-[2-(3,5- | secretase |
| | Difluorophenyl)acetylamino]- | |
| | N-(5-methyl-6-oxo-6,7- | |
| | dihydro--5H- | |
| | dibenzo[b,d]azepin-7- | |
| | yl)propionamide | |
| γ-secretase inhibitor | (S,S)-2-[2-(3,5- | Inhibition of Notch cleavage by γ- |
| XXI (GSI XXI) | Difluorophenyl)- | secretase |
| | acetylamino]-N-(1-methyl-2- | |
| | oxo-5-phenyl-2-,3-dihydro- | |
| | 1H-benzo[e][1,4]diazepin-3- | |
| | yl)-propionamide | |
| Gamma40 secretase | N-trans-3,5- | Inhibition of Notch cleavage by γ- |
| inhibitor I | Dimethoxycinnamoyl-Ile- | secretase |
| | leucinal | |
| Gamma40 secretase | N-tert-Butyloxycarbonyl- | Inhibition of Notch cleavage by γ- |
| inhibitor II | Gly-Val-Valinal | secretase |
| | Isovaleryl-V V-Sta-A-Sta- | Inhibition of Notch cleavage by γ- |
| | OCH₃ | secretase |
| MK-0752 (Merck) | | Inhibition of Notch cleavage by γ- |
| | | secretase |
| LY450139 (Eli Lilly) | | Inhibition of Notch cleavage by γ- |
| | | secretase |
| RO4929097 | | Inhibition of Notch cleavage by γ- |
| | | secretase |
| PF-03084,014 | | Inhibition of Notch cleavage by γ- |
| | | secretase |
| BMS-708163 | | Inhibition of Notch cleavage by γ- |
| | | secretase |
| MPC-7869 (γ-secretase | | Inhibition of Notch cleavage by γ- |
| modifier) | | secretase |
| MAML-CSL-Notch | | Interference with Notch nuclear co- |
| | | activator MAML1 |
| Antennapedia/dominant- | | Interference with Notch nuclear co- |
| MAML | | activator MAML1 |
| OMP-21M18 (DLL4 | | Interference with Dll4 ligand- |
| antibody) | | receptor interaction |
| Notch soluble receptor | | |
| decoys | | |

As examples of the use of γ-secretase inhibitors, the γ-secretase inhibitor MK-0752 (Merck) has been administered to human subjects in single doses of 110 to 1000 mg (Rosen et al., 2006). MK-0752 is in Phase I clinical trials for patients with breast cancer tumors (ClinicalTrials.gov Identifier NCT00106145). The γ-secretase inhibitor LY450139 (Eli Lilly) has been administered to human subjects at doses ranging from 5 mg/day to 50 mg/day for 14 days (Seimers et al., 2005). A longer term study with LY450139 has been conducted at a dose of 60 mg/day for 2 weeks, followed by 100 mg/day for 6 weeks, followed by either 100 mg/day or 140 mg/day for another 6 weeks (Beals, 2007).

Combination Therapy

Therapeutic compositions can be administered in therapeutically effective dosages alone or in combination with adjuvant cancer therapy such as surgery, chemotherapy, radiotherapy, thermotherapy, and laser therapy, and may provide a beneficial effect, e.g. reducing tumor size, slowing rate of tumor growth, inhibiting metastasis, or otherwise improving overall clinical condition, without necessarily eradicating the cancer. Notch-targeted therapies appear to exert beneficial effects by targeting tumor vasculature. Cytostatic and cytotoxic agents that target the cancer cells are specifically contemplated for combination therapy. Likewise, agents that target angiogenesis or lymphangiogenesis are specifically contemplated for combination therapy.

A. Chemotherapy

As used herein, a “chemotherapeutic agent” is a chemical compound useful in the treatment of cancer. Examples of chemotherapeutic agents include: alkylating agents such as thiotepa and CYTOXAN® cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethiylenethiophosphoramide and tiimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; vinca alkaloids; epipodophyllotoxins; antibiotics such as the enediyne antibiotics (e.g., calicheamicin, especially calicheamicin gammall and calicheamicin omegall; L-asparaginase; anthracenedione substituted urea; methyl hydrazine derivatives; dynemicin, including dynemicin A; bisphosphonates, such as clodronate; an esperamicin; as well as neocarzinostatin chromophore and related chromoprotein enediyne antiobiotic chromophores), aclacinomysins, actinomycin, authramycin, azaserine, bleomycins, cactinomycin, carabicin, caminomycin, carzinophilin, chromomycinis, dactinomycin, daunorubicin, detorubicin, 6-diazo-5-oxo-L-norleucine, ADRIAMYCIN® doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogs such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitiaerine; pentostatin; phenamet; pirarubicin; losoxantione; podophyllinic acid; 2-ethylhydrazide; procarbazine; PSK® polysaccharide complex (JHS Natural Products, Eugene, Oreg.); razoxane; rhizoxin; sizofuran; spirogermanium; tenuazonic acid; triaziquone; 2,2 2″-trichlorotiiethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g., TAXOL® paclitaxel (Bristol-Myers Squibb Oncology, Princeton, N.J.), ABRAXANE™ Cremophor-free, albumin-engineered nanoparticle formulation of paclitaxel (American Pharmaceutical Partners, Schaumberg, Ill.), and TAXOTERE® docetaxel (Rhône-Poulenc Rorer, Antony, France); chloranbucil; GEMZAR® gemcitabine; 6-thioguanine; mercaptopurine; methotrexate; platinum coordination complexes such as cisplatin, oxaliplatin and carboplatin; vinblastine; platinum; etoposide (VP-16); ifosfamide; mitoxantrone; vincristine; NAVELBINE® vinorelbine; novantrone; teniposide; edatrexate; daunomycin; aminopterin; xeloda; ibandronate; irinotecan (e.g., CPT-11); topoisomerase inhibitor RFS 2000; difluoromethylornithine (DFMO); retinoids such as retinoic acid; capecitabine; leucovorin (LV); irenotecan; adrenocortical suppressant; adrenocorticosteroids; progestins; estrogens; androgens; gonadotropin-releasing hormone analogs; and pharmaceutically acceptable salts, acids or derivatives of any of the above. Also included in this definition are anti-hormonal agents that act to regulate or inhibit hormone action on tumors such as anti-estrogens and selective estrogen receptor modulators (SERMs), including, for example, tamoxifen (including NOLVADEX® tamoxifen), raloxifene, droloxifene, 4-hydroxytamoxifen, trioxifene, keoxifene, LY 117018, onapristone, and FARESTON-toremifene; aromatase inhibitors that inhibit the enzyme aromatase, which regulates estrogen production in the adrenal glands, such as, for example, 4(5)-imidazoles, aminoglutethimide, MEGASE® megestrol acetate, AROMASL® exemestane, formestanie, fadrozole, RIVISOR® vorozole, FEMARA® letrozole, and ARTMIDEX® anastrozole; and anti-androgens such as flutamide, nilutamide, bicalutamide, leuprolide, and goserelin; as well as troxacitabine (a 1,3-dioxolane nucleoside cytosine analog); antisense oligonucleotides, particularly those which inhibit expression of genes in signaling pathways implicated in abherant cell proliferation, such as, for example, PKC-alpha, Ralf and H-Ras; ribozymes such as a VEGF-A expression inhibitor (e.g., ANGIOZYME® ribozyme) and a HER2 expression inhibitor; vaccines such as gene therapy vaccines, for example, ALLOVECTIN® vaccine, LEUVECTIN® vaccine, and VAXID® vaccine; PROLEUKIN® rJL-2; LURTOTECAN® topoisomerase 1 inhibitor; ABARELLX® rmRH; and pharmaceutically acceptable salts, acids or derivatives of any of the above.

A “growth inhibitory agent” as used herein refers to a compound or composition which inhibits growth of a cell in vitro and/or in vivo. Thus, the growth inhibitory agent may be one which significantly reduces the percentage of cells in S phase. Examples of growth inhibitory agents include agents that block cell cycle progression (at a place other than S phase), such as agents that induce G1 arrest and M-phase arrest. Classical M-phase blockers include the vincas (vincristine and vinblastine), TAXOL®, and topo II inhibitors such as doxorubicin, epirubicin, daunorubicin, etoposide, and bleomycin. Those agents that arrest G1 also spill over into S-phase arrest, for example, DNA alkylating agents such as tamoxifen, prednisone, dacarbazine, mechlorethamine, cisplatin, methotrexate, 5-fluorouracil, and ara-C.

Also disclosed is a method of treating a human subject susceptible to or diagnosed with colorectal cancer, pancreatic cancer, prostate cancer or glioblastoma, comprising administering in combination to the subject effective amounts of Notch inhibitor described herein and a standard of care chemotherapy for colorectal cancer, pancreatic cancer, prostate cancer or glioblastoma cancer. The standard chemotherapy for colorectal cancer, pancreatic cancer, prostate cancer or glioblastoma comprises 5-FU, gemcitabine, docetaxel, and TMZ, respectively. The colorectal cancer, pancreatic cancer, prostate cancer or glioblastoma can be metastatic.

By way of example only, standard chemotherapy treatments for metastatic colorectal cancer are described herein below. The standard treatment for metastatic colorectal cancer in the United States has been until recently chemotherapy with 5-FU plus a biochemical modulator of 5-FU, leucovorin. The combination of 5-FU/leucovorin provides infrequent, transient shrinkage of colorectal tumors but has not been demonstrated to prolong survival compared with 5-FU alone, and 5-FU has not been demonstrated to prolong survival compared with an ineffective therapy plus best supportive care. The lack of a demonstrated survival benefit for 5-FU/leucovorin may be due in part to inadequately sized clinical trials. In a large randomized trial of subjects receiving adjuvant chemotherapy for resectable colorectal cancer, 5-FU/leucovorin demonstrated prolonged survival compared with lomustine (MeCCNU), vincristine, and 5-FU (MOF).

In the United States, 5-FU/leucovorin chemotherapy is commonly administered according to one of two schedules: the Mayo Clinic and Roswell Park regimens. The Mayo Clinic regimen consists of an intensive course of 5-FU plus low-dose leucovorin (425 mg/m² 5-FU plus 20 mg/m² leucovorin administered daily by intravenous (IV) push for 5 days, with courses repeated at 4- to 5-week intervals). The Roswell Park regimen consists of weekly 5-FU plus high-dose leucovorin (500-600 mg/m² 5-FU administered by IV push plus 500 mg/m² leucovorin administered as a 2-hour infusion weekly for 6 weeks, with courses repeated every 8 weeks). Clinical trials comparing the Mayo Clinic and Roswell Park regimens have not demonstrated a difference in efficacy, but have been underpowered to do so. The toxicity profiles of the two regimens are different, with the Mayo Clinic regimen resulting in more leukopenia and stomatitis and the Roswell Park regimen resulting in more frequent diarrhea. Subjects with newly diagnosed metastatic colorectal cancer receiving either regimen can expect a median time to disease progression of 4-5 months and a median survival of 12-14 months.

Recently, a new first-line therapy for metastatic colorectal cancer has emerged. Two randomized clinical trials, each with approximately 400 subjects, evaluated irinotecan in combination with 5-FU/leucovorin. In both studies, the combination of irinotecan/5-FU/leucovorin demonstrated statistically significant increases in survival (of 2.2 and 3.3 months), time to disease progression and response rates as compared with 5-FU/leucovorin alone. The benefits of irinotecan came at a price of increased toxicity: addition of irinotecan to 5-FU/leucovorin was associated with an increased incidence of National Cancer Institute Common Toxicity Criteria (NCI-CTC) Grade 3/4 diarrhea, Grade 3/4 vomiting, Grade 4 neutropenia, and asthenia compared with 5-FU/leucovorin alone. There is also evidence showing that single-agent irinotecan prolongs survival in the second-line setting. Two randomized studies have demonstrated that irinotecan prolongs survival in subjects who have progressed following 5-FU therapy. One study compared irinotecan to best supportive care and showed a 2.8-month prolongation of survival; the other study compared irinotecan with infusional 5-FU and showed a 2.2-month prolongation of survival. The question of whether irinotecan has more effect on survival in the first- or second-line setting has not been studied in a well-controlled fashion.

B. Anti-Angiogenesis Agents

In some embodiments, the methods described herein further comprises administering a molecule that inhibits angiogenesis, especially molecules that disrupt signaling between members of the VEGF/PDGF Family of growth factors and their cognate VEGF/PDGF receptors.

Exemplary molecules that inhibit angiogenesis include, but are not limited to, molecules that inhibit VEGFR-2 (i.e., VEGFR-2 inhibitor products). The “VEGFR-2 inhibitor product” can be any molecule that acts with specificity to reduce VEGF-C/VEGFR-2, VEGF-D/VEGFR-2 or VEGF/VEGFR-2 interactions, e.g., by blocking VEGF-C or VEGF-D binding to VEGFR-2, by blocking VEGF binding to VEGFR-2 or by reducing expression of VEGFR-2. In one embodiment, the VEGFR-2 inhibitor inhibits VEGF-C and VEGF-D binding to VEGFR-2. In another embodiment, the VEGFR-2 inhibitor inhibits binding of VEGF to VEGFR-2. The VEGFR-2 inhibitor can be a polypeptide comprising a soluble VEGFR-2 ECD fragment (amino acids 20-764 of SEQ ID NO: 22) that binds VEGF or VEGF-C or VEGF-D; VEGFR-2 anti-sense polynucleotides or short-interfering RNA (siRNA); anti-VEGFR-2 antibodies; a VEGFR-2 inhibitor polypeptide comprising an antigen-binding fragment of an anti-VEGFR-2 antibody that inhibits binding between VEGFR-2 and VEGF or VEGF-C or VEGF-D; an aptamer that inhibits binding between VEGFR-2 and VEGF; an aptamer that inhibits binding between VEGFR-2 and VEGF-C; an aptamer that inhibits binding between VEGFR-2 and VEGF-D; or a fusion protein comprising the soluble VEGFR-2 polypeptide fragment fused to an immunoglobulin constant region fragment (Fc). In some embodiments, a VEGFR-2 polypeptide fragment is fused to alkaline phosphatase (AP).

In other embodiments, the molecule that inhibits angiogenesis is Avastin (bevacizumab; Genetech/Roche). Bevacizumab was approved by the FDA in February 2004 for use in metastatic colorectal cancer when used with standard chemotherapy treatment (as first-line treatment) and with 5-fluorouracil-based therapy for second-line metastatic colorectal cancer. This recommendation was based on E3200 trial—addition of bevacizumab to oxaliplatin/5-FU/leucovorin (FOLFOX4) therapy. It was approved by the EMEA in January 2005 for use in colorectal cancer.

C. Inhibitors of VEGF-C/VEGFR-3 Signaling

In some embodiments, the methods described herein further comprise administering a molecule that inhibits VEGF-C/VEGFR-3 signaling in the treatment of the cancer. This ligand receptor pair has been shown to play a major role in lymphangiogenesis and also play a role in pathogenic angiogenesis, e.g., in some cancers. Suitable molecules include, but are not limited to, an antibody that binds VEGF-C and inhibits VEGF-C stimulation of VEGFR-3; or a fragment of said antibody that retains the inhibitory activity; an antibody that binds VEGFR-3 and inhibits VEGF-C stimulation of VEGFR-3; or a fragment of said antibody that retains the inhibitory activity; an antibody that binds VEGFR-2 and inhibits VEGF-C stimulation of VEGFR-2; or a fragment of said antibody that retains the inhibitory activity; a polypeptide that comprises a VEGFR-3 ECD fragment that binds VEGF-C and inhibits VEGF-C stimulation of VEGFR-3; an antisense or interfering nucleic acid (e.g., antisense oligonucleotide; micro-RNA, short interfering RNA) that inhibits VEGF-C expression; an antisense or interfering nucleic acid that inhibits VEGFR-3 expression; and a polypeptide that comprises a VEGFR-2 ECD fragment that binds VEGF-C and inhibits VEGF-C stimulation of VEGFR-3.

In some embodiments, the molecule comprises short interfering RNA (siRNA) that inhibits VEGF-C expression. RNA interference of the VEGF family of proteins and receptors is described in U.S. Patent application Publication Nos.: 2006/0217332, 2006/0025370, 2005/0233998, 2005/0222066 and 2005/0171039, the disclosure of which are incorporated herein by reference in their entireties. Interfering RNA directed to VEGF or VEGFR family members is described in U.S. Patent Publication No. 2006/0217332, incorporated herein by reference.

In some embodiments, the molecule comprises a zinc finger protein that inhibits VEGF-C expression.

Exemplary anti-VEGFR-3 antibodies and their production are described in U.S. Pat. Nos. 6,107,046 and 6,824,777; U.S. Patent Publication Nos. 2006/0269548 and 2006/0177901; and International Patent Application No. PCT/FI95/00337 (WO 95/33772), all incorporated herein by reference in their entireties.

Exemplary VEGF-C antibodies are described, for example, in International Patent Application Nos. PCT/FI1996/000427 (WO/1997/005250) and PCT/US1998/001973 (WO/1998/033917); and U.S. Patent Publication Nos. 2004/0147726, 2005/0232921, 2005/0192429, 2005/0059117, 2005/0282228, 2003/0176674, and 2006/0121025, 2006/0030000, and U.S. Pat. No. 6,403,088 all incorporated herein by reference.

Exemplary anti-VEGFR antibodies and other inhibitor compounds are described, for example, in U.S. Pat. Nos. 7,056,509; 7,052,693; 6,986,890; 6,897,294; 6,887,468; 6,878,720; 6,344,339; 5,955,311; 5,874,542; and 5,840,301, all incorporated herein by reference.

D. Small Molecule Inhibitors

Any chemical substance that can be safely administered as a therapeutic and that can be used to modulate biochemical pathway targets identified herein, such as VEGF-mediated stimulation of VEGF receptors, may be used to practice the invention. Small molecules that inhibit the interaction between VEGF-C and/or VEGFR-3 with VEGFR-3 are specifically contemplated. VEGF-C/VEGF-D inhibitors are disclosed in U.S. Pat. No. 7,045,133, incorporated herein by reference.

That patent describes, for example, monomeric monocyclic peptide inhibitors based on loop 1, 2 or 3 of VEGF-D. A preferred peptide interferes with at least the activity of VEGF-D and VEGF-C mediated by VEGF receptor-2 and VEGF receptor-3 (VEGFR-3). A particularly preferred peptide interferes with the activity of VEGF-D, VEGF-C and VEGF mediated by VEGFR-2 and the activity of VEGF-D and VEGF-C mediated by VEGFR-3. The patent also describes a dimeric bicyclic peptide inhibitor which comprises two monomeric monocyclic peptides, each individually based on loop 1, 2 or 3 of VEGF-D, linked together. Such dimeric bicyclic peptides may comprise two monomeric monocyclic peptides which are the same or different. (See, for example, Table 1-3 of the '133 patent.)

The VEGF receptors are receptor tyrosine kinases and intracellular signaling is initiated through receptor phosphorylation. Accordingly, one preferred class of molecules for practice of the invention is tyrosine kinase inhibitors, including those described in and Morin,

Oncogene, 19(56):6574-83, 2000, incorporated herein by reference. VEGFR-3 inhibitors are disclosed in U.S. Patent Publication No. 2002-0164667, incorporated herein by reference.

E. Tyrosine Kinase Inhibitors

In another embodiment, the methods described herein optionally further comprise administering a tyrosine kinase inhibitor that inhibits VEGFR-2 and/or VEGFR-3 activity.

Exemplary tyrosine kinase inhibitors for use in the methods described herein include, but are not limited to, AEE788 (TKI, VEGFR-2, EGFR: Novartis); ZD6474 (TKI, VEGFR-1, -2, -3, EGFR: Zactima: AstraZeneca); AZD2171 (TKI, VEGFR-1, -2: AstraZeneca); SU 11248 (TKI, VEGFR-1, -2, PDGFR: Sunitinib: Pfizer); AG13925 (TKI, VEGFR-1, -2: Pfizer); AG013736 (TKI, VEGFR-1, -2: Pfizer); CEP-7055 (TKI, VEGFR-1, -2, -3: Cephalon); CP-547,632 (TKI, VEGFR-1, -2: Pfizer); GW7S6024 (TKL VEGFR-1, -2, -3: GlaxoSmithKline); GW786034 (TKI, VEGFR-1, -2, -3: GlaxoSmithKline); sorafenib (TKI, Bay 43-9006, VEGFR-1, -2, PDGFR: Bayer/Onyx); SU4312 (TKI, VEGFR-2, PDGFR: Pfizer); AMG706 (TKI, VEGFR-1, -2, -3: Amgen); XL647 (TKI, EGFR, HER2, VEGFR, ErbB4: Exelixis); XL999 (TK1, FGFR, VEGFR, PDGFR, F11-3: Exelixis); PKC412 (TKI, KIT, PDGFR, PKC, FLT3, VEGFR-2: Novartis); AEE788 (TKI, EGFR, VEGFR2, VEGFR-1: Novartis): OSI-030 (TKI, c-kil, VEGFR: OSI Pharmaceuticals); OS1-817 (TKI c-kit, VEGFR: OSI Pharmaceuticals); DMPQ (TKI, ERGF, PDGFR, ErbB2. p56. pkA, pkC); MLN518 (TK1, Flt3, PDGFR, c-KIT (T53518: Millennium Pharmaceuticals); lestaurinib (TKI, FLT3, CEP-701, Cephalon); ZD 1839 (TKI, EGFR: gefitinib, Iressa: AstraZcneca); OSI-774 (TKI, EGFR: Erlotininb: Tarceva: OSI Pharmaceuticals); lapatinib (TKI, ErbB-2, EGFR, and GD-2016: Tykerb: GlaxoSmithKline).

In some embodiments, the methods described herein further comprise administering a tyrosine kinase inhibitor that inhibits angiogenesis to the subject. Exemplary anti-angiogenic tyrosine kinase inhibits and their targets are provided below in Table 2.

TABLE 2
Antiangiogenic tyrosine kinase receptor inhibitors and their targets

| Agent | VEGFR-1 | VEGFR-2 | VEGFR-3 | PDGFR | EGFR | Other targets |
|---|---|---|---|---|---|---|
| Vandetanib | | • | | | • | RET |
| Sunitinib | • | • | • | • | | KIT, FLT3, RET |
| Axitinib | • | • | • | | | |
| Sorafenib | • | • | • | • | | KIT, RAF, FLT3 |
| Vatalanib | • | • | • | • | | KIT |
| Cediranib | • | • | • | • | | KIT |
| Motesanib | • | • | • | • | | KIT, RET |
| Pazopanib | • | • | • | • | | KIT |
| BIBF 1120 | | • | | • | | FGFR |

Abbreviations:
FGFR, fibroblast-like growth factor receptor;
FLT3, FMS-like tyrosine kinase 3;
KIT, stem cell factor receptor;
RET, glial cell line-derived neurotrophic factor receptor;
VEGFR, vascular endothelial growth factor receptor.

F. Inhibitors of the PI3/AKT/mTOR Pathway

In some embodiments, the methods described herein further comprise administering an inhibitor of the PI3/AKT/mTOR pathway. In some embodiments, the inhibitor is a PI3 kinase inhibitor selected from the group consisting of wortmannin (Calbiochem), 3-methyladenine (3-MA, Sigma), LY294002 (Calbiochem) and chloroquine.

In some embodiments, the inhibitor is a mTOR (mammalian target of rapamycin) inhibitor. Exemplary mTOR inhibitors include, but are not limited to, the following:

| Brand Name or | | Patent (U.S. Unless Specified) |
|---|---|---|
| Product # | Generic Name | or Reference |
| Rapamune ® | rapamycin (sirolimus) | 3,929,992; 5,288,711; |
| RAD001 | everolimus; 40-O-(2- | 5,516,781 EP663916; |
| (Certican ®) | hydroxyethyl)-rapamycin | U.S. Pat. Appl. 20030170287 |
| CCI-779 | Rapamycin 42-ester with | 6,617,333; 5,362,718; |
| | 3-hydroxy-2- | 6,277,983 |
| | (hydroxymethyl)-2- | |
| | methylpropionic acid | |
| | Tumstatin and related | U.S. Pat. Appl. 20030144481 |
| | polypeptides | |
| ABT578 | | U.S. Pat. Appl. 20030073737 |
| “rapalogs,” | | U.S. Pat. Appl. 20030073737; |
| e.g., AP23573, | | WO01/02441; WO01/14387 |
| AP22594 | | |
| AP23841 | | ARIAD Pharmaceuticals |
| TAFA93 | | Isotechnika |

In addition to rapamycin and those derivatives of rapamycin listed in the above table those discussed in U.S. Pat. Appl. No. 20030170287 may also be used. See also WO 94/09010, and WO 96/41807. Rapamycin derivatives may also include without limitation “rapalogs,” e.g., as disclosed in WO 98/02441 and WO01/14387; deuterated rapamycin analogs, e.g., as disclosed in U.S. Pat. No. 6,503,921. Derivatives of other mTOR inhibitors are also contemplated.

G. Administration of the Combination Therapy

Combination therapy with one or more of the additional agents described herein may be achieved by administering to a subject a single composition or pharmacological formulation that includes the Notch inhibitor and the one or more additional agents, or by administering to the subject two (or more) distinct compositions or formulations, at the same time, wherein one composition includes a Notch inhibitor and the other includes a second agent.

Alternatively, the combination therapy employing a Notch inhibitor described herein may precede or follow the second agent treatment by intervals ranging from minutes to weeks. In embodiments where the second agent and the Notch inhibitor are administered separately, one would generally ensure that a significant period of time did not expire between the times of each delivery, such that the agent and the Notch inhibitor would still be able to exert an advantageously combined effect. In such instances, it is contemplated that one would administer both modalities within about 12-24 hours of each other and, more preferably, within about 6-12 hours of each other, with a delay time of only about 12 hours being most preferred. In some situations, it may be desirable to extend the time period for treatment significantly, however, where several days (2, 3, 4, 5, 6 or 7) to several weeks (1, 2, 3, 4, 5, 6, 7 or 8) lapse between the respective administrations. Repeated treatments with one or both agents is specifically contemplated.

### Pharmaceutical Compositions and Routes of Administration

Purified nucleic acids, antisense molecules, purified protein, antibodies, antagonists, or inhibitors may all be used as pharmaceutical compositions. Delivery of specific molecules for therapeutic purposes in this invention is further described below.

The active compositions of the present invention include classic pharmaceutical preparations. Administration of these compositions according to the present invention will be via any common route so long as the target tissue is available via that route. The pharmaceutical compositions may be introduced into the subject by any conventional method, e.g., by intravenous, intradermal, intramusclar, intramammary, intraperitoneal, intrathecal, intraocular, retrobulbar, intrapulmonary (e.g., term release); by oral, sublingual, nasal, anal, vaginal, or transdermal delivery, or by surgical implantation at a particular site, e.g., embedded under the splenic capsule, brain, or in the cornea. The treatment may consist of a single dose or a plurality of doses over a period of time.

For all protein-based therapeutics described herein (e.g., soluble receptors or antibodies) administration by the delivery of gene expression constructs are contemplated as one embodiment. Any suitable vector may be used to introduce a polynucleotide that encodes a protein-based therapeutic described herein, into the host. Exemplary vectors that have been described in the literature include replication deficient retroviral vectors, including but not limited to lentivirus vectors [Kim et al., 1998; Kingsman and Johnson, 1998]; adeno-associated viral (AAV) vectors [U.S. Pat. No. 5,474,935; U.S. Pat. No. 5,139,941; U.S. Pat. No. 5,622,856; U.S. Pat. No. 5,658,776; U.S. Pat. No. 5,773,289; U.S. Pat. No. 5,789,390; U.S. Pat. No. 5,834,441; U.S. Pat. No. 5,863,541; U.S. Pat. No. 5,851,521; U.S. Pat. No. 5,252,479; Gnatenko et al., 1997]; adenoviral (AV) vectors [See, e.g., U.S. Pat. No. 5,792,453; U.S. Pat. No. 5,824,544; U.S. Pat. No. 5,707,618; U.S. Pat. No. 5,693,509; U.S. Pat. No. 5,670,488; U.S. Pat. No. 5,585,362; Quantin et al., 1992; Stratford Perricadet et al., 1992; and Rosenfeld et al., 1992]; an adenoviral adenoassociated viral chimeric (see for example, U.S. Pat. No. 5,856,152) or a vaccinia viral or a herpesviral (see for example, U.S. Pat. No. 5,879,934; U.S. Pat. No. 5,849,571; U.S. Pat. No. 5,830,727; U.S. Pat. No. 5,661,033; U.S. Pat. No. 5,328,688; Lipofectin mediated gene transfer (BRL); liposomal vectors [See, e.g., U.S. Pat. No. 5,631,237 (Liposomes comprising Sendai virus proteins)]; and combinations thereof. All of the foregoing documents are incorporated herein by reference in their entirety. Replication deficient adenoviral vectors constitute a preferred embodiment.

The active compounds may be prepared for administration as solutions of free base or pharmacologically acceptable salts in water suitably mixed with a surfactant, such as hydroxypropylcellulose. Dispersions also can be prepared in glycerol, liquid polyethylene glycols, and mixtures thereof and in oils. Under ordinary conditions of storage and use, these preparations contain a preservative to prevent the growth of microorganisms.

The pharmaceutical forms suitable for injectable use include sterile aqueous solutions or dispersions and sterile powders for the extemporaneous preparation of sterile injectable solutions or dispersions. In all cases the form must be sterile and must be fluid to the extent that easy syringability exists. It must be stable under the conditions of manufacture and storage and must be preserved against the contaminating action of microorganisms, such as bacteria and fungi. The carrier can be a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

As used herein, “pharmaceutically acceptable carrier” includes any and all solvents, dispersion media, coatings, antibacterial and antifungal agents, isotonic and absorption delaying agents and the like. The use of such media and agents for pharmaceutical active substances is well known in the art. Except insofar as any conventional media or agent is incompatible with the active ingredient, its use in the therapeutic compositions is contemplated. Supplementary active ingredients also can be incorporated into the compositions.

For oral administration the active compositions may be incorporated with excipients and used in the form of non-ingestible mouthwashes and dentifrices. A mouthwash may be prepared incorporating the active ingredient in the required amount in an appropriate solvent, such as a sodium borate solution (Dobell's Solution). Alternatively, the active ingredient may be incorporated into an antiseptic wash containing sodium borate, glycerin and potassium bicarbonate. The active ingredient may also be dispersed in dentifrices, including: gels, pastes, powders and slurries. The active ingredient may be added in a therapeutically effective amount to a paste dentifrice that may include water, binders, abrasives, flavoring agents, foaming agents, and humectants.

The compositions of the present invention may be formulated in a neutral or salt form. Pharmaceutically-acceptable salts include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups also can be derived from inorganic bases such as, for example, sodium, potassium, ammonium, calcium, or ferric hydroxides, and such organic bases as isopropylamine, trimethylamine, histidine, procaine and the like.

The compositions described herein may be formulated in a neutral or salt form. Pharmaceutically-acceptable salts include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups also can be derived from inorganic bases such as, for example, sodium, potassium, ammonium, calcium, or ferric hydroxides, and such organic bases as isopropylamine, trimethylamine, histidine, procaine and the like.

Upon formulation, solutions will be administered in a manner compatible with the dosage formulation and in such amount as is therapeutically effective. The formulations are easily administered in a variety of dosage forms such as injectable solutions, drug release capsules and the like. For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous and intraperitoneal administration.

In the clinical setting an “effective amount” is an amount sufficient to effect beneficial or desired clinical results. An effective amount can be administered in one or more doses. In terms of treatment, an “effective amount” of a therapeutic agent described herein is an amount that results in amelioration of symptoms or a prolongation of survival in a subject. The effective amount is generally determined by the physician on a case-by-case basis and is within the skill of one in the art. Several factors are typically taken into account when determining, an appropriate dosage. These factors include age, sex and weight of the patient, the condition being treated, the severity of the condition. Determination of the effective amounts is well within the capability of those skilled in the art, especially in light of the detailed disclosure provided herein. For any therapeutic agent used in a method described herein, the therapeutically effective dose can be estimated initially from cell culture assays. For example, a dose can be formulated in animal models to achieve a circulating concentration range that includes the IC₅₀ as determined in cell culture (i.e., the concentration of the test compound which achieves a half-maximal inhibition of the C-proteinase activity). Such information can be used to more accurately determine useful doses in humans.

Toxicity and therapeutic efficacy of such compounds can be determined by standard pharmaceutical procedures in cell cultures or experimental animals, e.g., for determining the LD₅₀ (the dose lethal to 50% of the population) and the ED50 (the dose therapeutically effective in 50% of the population). The dose ratio between toxic and therapeutic effects is the therapeutic index and it can be expressed as the ratio between LD50 and ED50. Compounds which exhibit high therapeutic indices are preferred. The data obtained from these cell culture assays and animal studies can be used in formulating a range of dosage for use in human. The dosage of such compounds lies preferably within a range of circulating concentrations that include the ED₅₀ with little or no toxicity. The dosage may vary within this range depending upon the dosage form employed and the route of administration utilized. The exact formulation, route of administration and dosage can be chosen by the individual physician in view of the patient's condition. See, e.g., Fingl and Woodbury, 1975. Dosage amount and interval may be adjusted individually to provide plasma levels of the active moiety which are sufficient to maintain the C-proteinase inhibiting effects, or minimal effective concentration (MEC). The MEC will vary for each compound but can be estimated from in vitro data; for example, the concentration necessary to achieve 50-90% inhibition of the C-proteinase using the assays described herein. Dosages necessary to achieve the MEC will depend on individual characteristics and route of administration. However, HPLC assays or bioassays can be used to determine plasma concentrations.

Dosage intervals can also be determined using MEC value. Compounds should be administered using a regimen which maintains plasma levels above the MEC for 10-90% of the time, preferably between 30-90% and most preferably between 50-90%. In cases of local administration or selective uptake, the effective local concentration of the drug may not be related to plasma concentration. Refinement of the calculations necessary to determine the appropriate treatment dose is routinely made by those of ordinary skill in the art without undue experimentation, especially in light of the dosage information and assays disclosed herein as well as the pharmacokinetic data observed in animals or human clinical trials. As studies are conducted, further information will emerge regarding appropriate dosage levels and duration of treatment for specific diseases and conditions.

In a preferred embodiment, the present invention is directed at treatment of a cancer selected from the group consisting of colorectal cancer, breast cancer, lung cancer, gastric cancer, glioblastoma and pancreatic cancer, wherein the cancer expresses an elevated level of VEGF-C. A variety of different routes of administration are contemplated. For example, in the case of a tumor, the discrete tumor mass may be injected with a therapeutic agent described herein. The injections may be single or multiple; where multiple, injections are made at about 1 cm spacings across the accessible surface of the tumor. Alternatively, targeting the tumor vasculature by direct, local or regional intra-arterial injection are contemplated. The lymphatic systems, including regional lymph nodes, present another likely target for delivery. Further, systemic injection may be preferred.

It will be appreciated that the pharmaceutical compositions and treatment methods of the invention may be useful in fields of human medicine and veterinary medicine. Thus the subject to be treated may be a mammal, preferably human or other animal. For veterinary purposes, subjects include for example, farm animals including cows, sheep, pigs, horses and goats, companion animals such as dogs and cats, exotic and/or zoo animals, laboratory animals including mice rats, rabbits, guinea pigs and hamsters; and poultry such as chickens, turkey ducks and geese.

The compositions may, if desired, be presented in a pack or dispenser device which may contain one or more unit dosage forms containing the active ingredient. The pack may, for example, comprise metal or plastic foil, such as a blister pack. The pack or dispenser device may be accompanied by instructions for administration. Compositions comprising a compound of the invention formulated in a compatible pharmaceutical carrier may also be prepared, placed in an appropriate container, and labeled for treatment of an indicated condition.

Kits

In another embodiment, kits are provided which contain the necessary reagents to carry out the assays described herein. Specifically, the invention provides a compartment kit to receive, in close confinement, one or more containers which comprises: (a) a first container comprising one of the probes or antibodies of the present invention; and (b) one or more other containers comprising one or more of the following: wash reagents, reagents capable of detecting presence of a bound probe or antibody (i.e., a VEGF-C antibody).

In detail, a compartment kit includes any kit in which reagents are contained in separate containers. Such containers include small glass containers, plastic containers, or strips of plastic or paper. Such containers allow one to efficiently transfer reagents from one compartment to another compartment such that the biological sample and reagents are not cross-contaminated, and the agents or solutions of each container can be added in a quantitative fashion from one compartment to another. Such containers will include a container which will accept the test sample, a container which contains, for example, the antibodies used in the assay, containers which contain wash reagents (such as phosphate buffered saline, Tris-buffers, etc.), and containers which contain the reagents used to detect the bound antibody or probe. Types of detection reagents include labeled nucleic acid probes, labeled secondary antibodies, or in the alternative, if the primary antibody is labeled, the enzymatic, or antibody binding reagents which are capable of reacting with the labeled antibody. One skilled in the art will readily recognize that the disclosed probes and antibodies of the present invention can be readily incorporated into one of the established kit formats which are well known in the art.

In further detail, kits for use in detecting the presence of a mammalian VEGF-C protein can include an antibody or functional fragment thereof which binds to a mammalian VEGF-C protein or portion of this protein, as well as one or more ancillary reagents suitable for detecting the presence of a complex between the antibody or fragment and VEGF-C or portion thereof. The antibody compositions o can be provided in lyophilized form, either alone or in combination with additional antibodies specific for other epitopes. The antibodies, which can be labeled or unlabeled, can be included in the kits with adjunct ingredients. For example, the antibodies can be provided as a lyophilized mixture with the adjunct ingredients, or the adjunct ingredients can be separately provided for combination by the user. Generally these adjunct materials will be present in less than about 5% weight based on the amount of active antibody, and usually will be present in a total amount of at least about 0.001% weight based on antibody concentration. Where a second antibody capable of binding to the monoclonal antibody is employed, such antibody can be provided in the kit, for instance in a separate vial or container. The second antibody, if present, is typically labeled, and can be formulated in an analogous manner with the antibody formulations described above.

Diagnostic Systems

Another aspect of the invention is a system that is useful for carrying out a part or all of a method of the invention, or carrying out a variation of a method of the invention as described herein in greater detail. Exemplary systems include, as one or more components, computing systems, environments, and/or configurations that may be suitable for implementation of, or use with, the methods and include, but are not limited to, personal computers, server computers, hand-held or laptop devices, multiprocessor systems, microprocessor-based systems, set top boxes, programmable consumer electronics, network PCs, minicomputers, mainframe computers, distributed computing environments that include any of the above systems or devices, and the like. In some variations, a system of the invention includes one or more machines used for analysis of biological material (e.g., genetic material), as described herein. In some variations, this analysis of the biological material involves a chemical analysis and/or a nucleic acid amplification or hybridization.

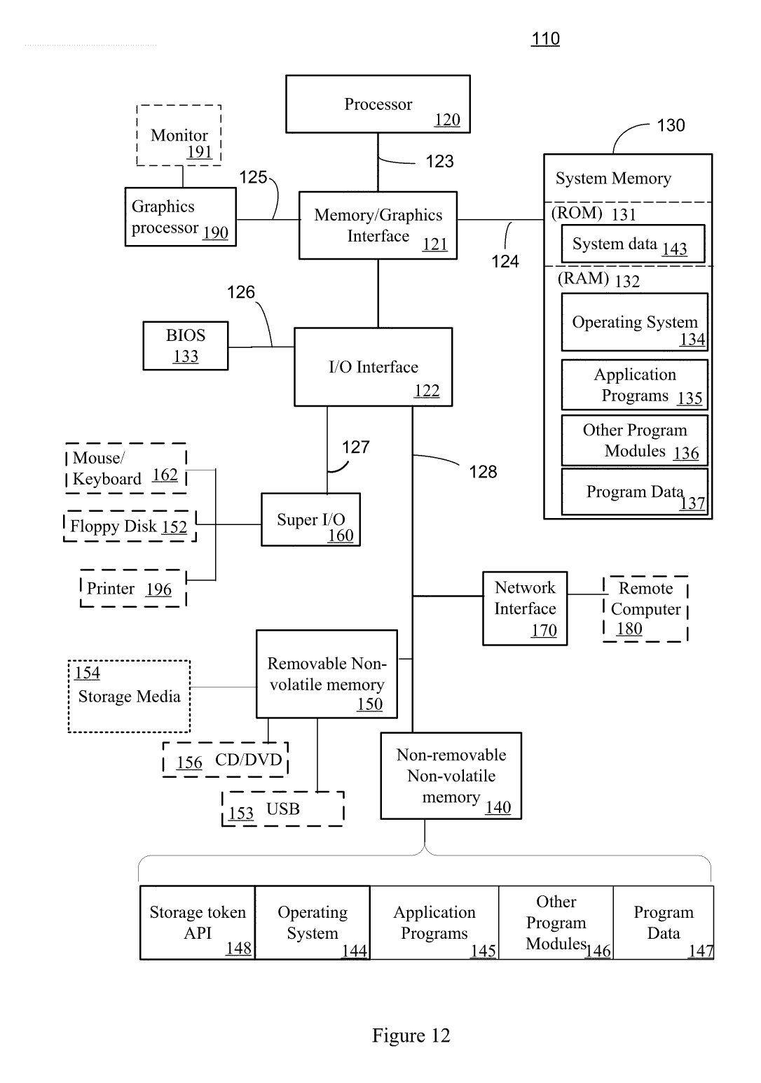

With reference to FIG. 12, an exemplary system of the invention, which may be used to implement one or more steps of methods of the invention, includes a computing device in the form of a computer 110. Components shown in dashed outline are not technically part of the computer 110, but are used to illustrate the exemplary embodiment of FIG. 12 Components of computer 110 may include, but are not limited to, a processor 120, a system memory 130, a memory/graphics interface 121, also known as a Northbridge chip, and an I/O interface 122, also known as a Southbridge chip. The system memory 130 and a graphics processor 190 may be coupled to the memory/graphics interface 121. A monitor 191 or other graphic output device may be coupled to the graphics processor 190.

A series of system busses may couple various system components including a high speed system bus 123 between the processor 120, the memory/graphics interface 121 and the I/O interface 122, a front-side bus 124 between the memory/graphics interface 121 and the system memory 130, and an advanced graphics processing (AGP) bus 125 between the memory/graphics interface 121 and the graphics processor 190. The system bus 123 may be any of several types of bus structures including, by way of example, and not limitation, such architectures include Industry Standard Architecture (USA) bus, Micro Channel Architecture (MCA) bus and Enhanced ISA (EISA) bus. As system architectures evolve, other bus architectures and chip sets may be used but often generally follow this pattern. For example, companies such as Intel and AMD support the Intel Hub Architecture (1HA) and the Hypertransport™ architecture, respectively.

The computer 110 typically includes a variety of computer-readable media. Computer-readable media can be any available media that can be accessed by computer 110 and includes both volatile and nonvolatile media, removable and non-removable media. By way of example, and not limitation, computer readable media may comprise computer storage media. Computer storage media includes both volatile and nonvolatile, removable and non-removable media implemented in any method or technology for storage of information such as computer readable instructions, data structures, program modules or other data. Computer storage media includes, but is not limited to, RAM, ROM, EEPROM, flash memory or other memory technology, CD-ROM, digital versatile disks (DVD) or other optical disk storage, magnetic cassettes, magnetic tape, magnetic disk storage or other magnetic storage devices, or any other physical medium which can be used to store the desired information and which can accessed by computer 110.

The system memory 130 includes computer storage media in the form of volatile and/or nonvolatile memory such as read only memory (ROM) 131 and random access memory (RAM) 132. The system ROM 131 may contain permanent system data 143, such as identifying and manufacturing information. In some embodiments, a basic input/output system (BIOS) may also be stored in system ROM 131. RAM 132 typically contains data and/or program modules that are immediately accessible to and/or presently being operated on by processor 120. By way of example, and not limitation, FIG. 12 illustrates operating system 134, application programs 135, other program modules 136, and program data 137.

The I/O interface 122 may couple the system bus 123 with a number of other busses 126, 127 and 128 that couple a variety of internal and external devices to the computer 110. A serial peripheral interface (SPI) bus 126 may connect to a basic input/output system (BIOS) memory 133 containing the basic routines that help to transfer information between elements within computer 110, such as during start-up.

A super input/output chip 160 may be used to connect to a number of ‘legacy’ peripherals, such as floppy disk 152, keyboard/mouse 162, and printer 196, as examples. The super I/O chip 160 may be connected to the I/O interface 122 with a bus 127, such as a low pin count (LPC) bus, in some embodiments. Various embodiments of the super I/O chip 160 are widely available in the commercial marketplace.

In one embodiment, bus 128 may be a Peripheral Component Interconnect (PCI) bus, or a variation thereof, may be used to connect higher speed peripherals to the I/O interface 122. A PCI bus may also be known as a Mezzanine bus. Variations of the PCI bus include the Peripheral Component Interconnect-Express (PCI-E) and the Peripheral Component Interconnect—Extended (PCI-X) busses, the former having a serial interface and the latter being a backward compatible parallel interface. In other embodiments, bus 128 may be an advanced technology attachment (ATA) bus, in the form of a serial ATA bus (SATA) or parallel ATA (PATA).

The computer 110 may also include other removable/non-removable, volatile/nonvolatile computer storage media. By way of example only, FIG. 12 illustrates a hard disk drive 140 that reads from or writes to non-removable, nonvolatile magnetic media. The hard disk drive 140 may be a conventional hard disk drive.

Removable media, such as a universal serial bus (USB) memory 153, firewire (IEEE 1394), or CD/DVD drive 156 may be connected to the PCI bus 128 directly or through an interface 150. A storage media 154 may coupled through interface 150. Other removable/non-removable, volatile/nonvolatile computer storage media that can be used in the exemplary operating environment include, but are not limited to, magnetic tape cassettes, flash memory cards, digital versatile disks, digital video tape, solid state RAM, solid state ROM, and the like.

The drives and their associated computer storage media discussed above and illustrated in FIG. 12, provide storage of computer readable instructions, data structures, program modules and other data for the computer 110. In FIG. 12, for example, hard disk drive 140 is illustrated as storing operating system 144, application programs 145, other program modules 146, and program data 147. Note that these components can either be the same as or different from operating system 134, application programs 135, other program modules 136, and program data 137. Operating system 144, application programs 145, other program modules 146, and program data 147 are given different numbers here to illustrate that, at a minimum, they are different copies. A user may enter commands and information into the computer 20 through input devices such as a mouse/keyboard 162 or other input device combination. Other input devices (not shown) may include a microphone, joystick, game pad, satellite dish, scanner, or the like. These and other input devices are often connected to the processor 120 through one of the I/O interface busses, such as the SPI 126, the LPC 127, or the PCI 128, but other busses may be used. In some embodiments, other devices may be coupled to parallel ports, infrared interfaces, game ports, and the like (not depicted), via the super I/O chip 160.

The computer 110 may operate in a networked environment using logical connections to one or more remote computers, such as a remote computer 180 via a network interface controller (NIC) 170. The remote computer 180 may be a personal computer, a server, a router, a network PC, a peer device or other common network node, and typically includes many or all of the elements described above relative to the computer 110. The logical connection between the NIC 170 and the remote computer 180 depicted in FIG. 12 may include a local area network (LAN), a wide area network (WAN), or both, but may also include other networks. Such networking environments are commonplace in offices, enterprise-wide computer networks, intranets, and the Internet. The remote computer 180 may also represent a web server supporting interactive sessions with the computer 110, or in the specific case of location-based applications may be a location server or an application server.

In some embodiments, the network interface may use a modem (not depicted) when a broadband connection is not available or is not used. It will be appreciated that the network connection shown is exemplary and other means of establishing a communications link between the computers may be used.

In some variations, the invention is a system for identifying a human subject with cancer for whom a Notch-targeted therapy will have efficacy. For example, in one variation, the system includes tools for performing at least one step, preferably two or more steps, and in some aspects all steps of a method of the invention, where the tools are operably linked to each other. Operable linkage describes a linkage through which components can function with each other to perform their purpose.

In some variations, a system of the invention is a system for identifying a human subject with cancer for whom a Notch-targeted therapy will have efficacy, the system comprising: (a) at least one processor; (b) at least one computer-readable medium; (c) a database operatively coupled to a computer-readable medium of the system and containing population information correlating the measurement of VEGF-C expression and efficacy of Notch-targeted therapy data in a population of humans with cancer; (d) a measurement tool that receives an input about the human subject and generates information from the input about the measurement of VEGF-C expression from the human subject; and (e) an analysis tool or routine that: (i) is operatively coupled to the database and the measurement tool, (ii) is stored on a computer-readable medium of the system, (iii) is adapted to be executed on a processor of the system, to compare the information about the human subject with the population information in the database and generate a conclusion with respect to a likelihood of efficacy of Notch-targeted therapy in the human subject.

Exemplary processors (processing units) include all variety of microprocessors and other processing units used in computing devices. Exemplary computer-readable media are described above. When two or more components of the system involve a processor or a computer-readable medium, the system generally can be created where a single processor and/or computer readable medium is dedicated to a single component of the system; or where two or more functions share a single processor and/or share a single computer readable medium, such that the system contains as few as one processor and/or one computer readable medium. In some variations, it is advantageous to use multiple processors or media, for example, where it is convenient to have components of the system at different locations. For instance, some components of a system may be located at a testing laboratory dedicated to laboratory or data analysis, whereas other components, including components (optional) for supplying input information or obtaining an output communication, may be located at a medical treatment or counseling facility (e.g., doctor's office, health clinic, HMO, pharmacist, geneticist, hospital) and/or at the home or business of the human subject (patient) for whom the testing service is performed.

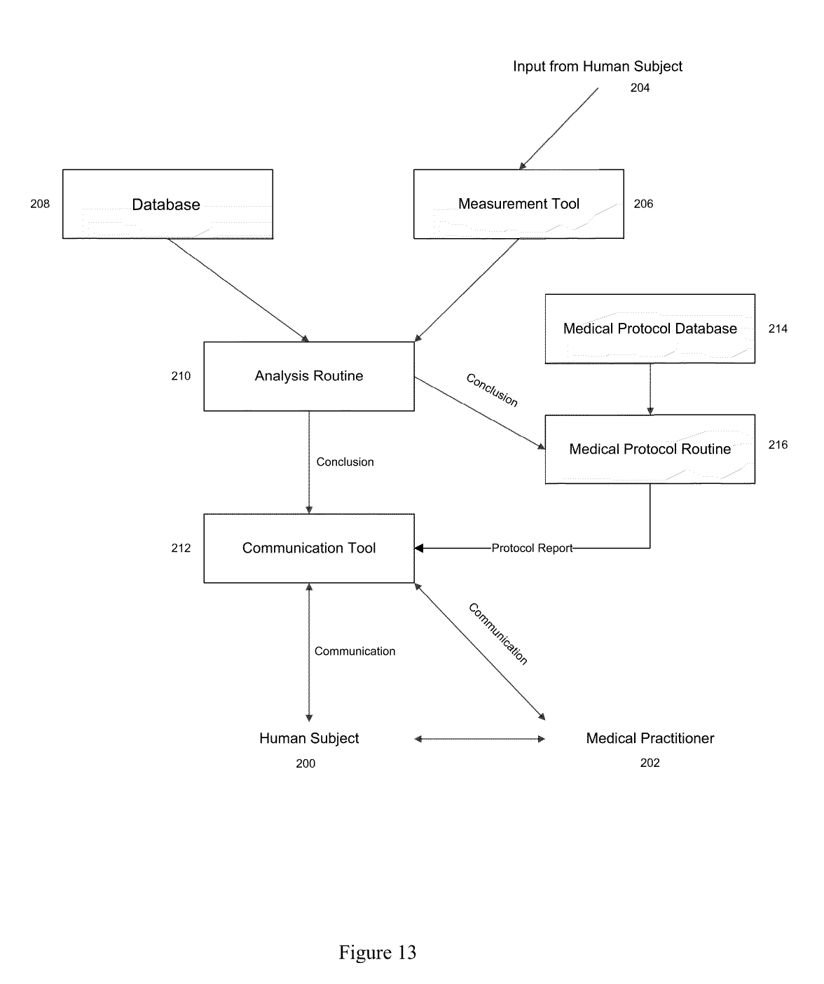

Referring to FIG. 13 an exemplary system includes a database 208 that is operatively coupled to a computer-readable medium of the system and that contains population information correlating the measurement of VEGF-C expression and efficacy of Notch-targeted therapy data in a population of humans. For example, for a statistically meaningful number of cancer subjects, information is collected as to whether the Notch-targeted therapy was effective in treating the cancer.

In a simple variation, the database contains 208 data relating to the level of VEGF-C expression observed in biological sample described herein of a population of humans with the cancer and information about the therapeutic efficacy of the Notch-targeted therapy. Such data provides an indication as to the likelihood of efficacy of Notch-targeted therapy in a human subject that is identified as having elevated VEGF-C expression. In still another variation, the database includes additional quantitative personal, medical, or genetic information about the individuals in the database with individuals having cancer that do not express elevated levels of VEGF-C (or control individuals free of the cancer). As the database becomes more populated with patient data, it becomes a more powerful statistical tool for comparing an input with respect to a subject and making a prediction as to the likelihood that Notch-targeted therapy would be an effective treatment. Such information includes, but is not limited to, information about parameters such as type of cancer, stage of cancer, age, sex, ethnicity, race, medical history, weight, diabetes status, blood pressure, family history of the cancer, smoking history, and alcohol use in humans. These more robust databases can be used by an analysis routine 210 to calculate a combined score with respect to potential for a cancer to metastasize.

In addition to the database 208, the system further includes a measurement tool 206 programmed to receive an input 204 from or about the human subject and generate an output that contains information about the measurement of VEGF-C expression. (The input 204 is not part of the system per se but is illustrated in the schematic FIG. 13.) Thus, the input 204 will contain a specimen or contain data from which a level of VEGF-C expression in a biological sample described herein can be directly read, or analytically determined.

In another variation, the input 204 from the human subject contains data that is unannotated or insufficiently annotated with respect to VEGF-C, requiring analysis by the measurement tool 206. For example, the input can be raw data measurements from experiments designed to evaluate the level of VEGF-C expression. In such variations, the measurement tool 206 comprises a tool, preferably stored on a computer-readable medium of the system and adapted to be executed on a processor of the system, to receive a data input about a subject and determine information about the level of VEGF-C expression from the data. For example, the measurement tool 206 contains instructions, preferably executable on a processor of the system, for analyzing the unannotated input data and determining the level of VEGF-C expression in the human subject.

In yet another variation, the input 204 from the human subject comprises a biological sample, such as a fluid (e.g., blood) or tissue sample, that contains one or more cancer cells or VEGF-C that can be analyzed to determine the expression level of the VEGF-C. In this variation, an exemplary measurement tool 206 includes laboratory equipment for processing and analyzing the sample to determine the level of VEGF-C expression in cancer cells of the human subject.

In some variations the measurement tool 206 includes: immunoassay reagents for measuring VEGF-C and measuring the level of VEGF-C expression from cells in the biological sample; and an analysis tool stored on a computer-readable medium of the system and adapted to be executed on a processor of the system, to determine the level of VEGF-C expression based on the immunoassay data.

In some variations, the measurement tool 206 further includes additional equipment and/or chemical reagents for processing the biological sample to purify VEGF-C from cells in a sample for further analysis using immunoassays, size separation tools, or other analytical equipment.

The exemplary system further includes an analysis tool or routine 210 that: is operatively coupled to the database 208 and operatively coupled to the measurement tool 206, is stored on a computer-readable medium of the system, is adapted to be executed on a processor of the system to compare the information about the human subject with the population information in the database 208 and generate a conclusion with respect to likelihood of efficacy of Notch-targeted therapy in the human subject. In simple terms, the analysis tool 210 looks at the level of VEGF-C expression identified by the measurement tool 206 for the human subject, and compares this information to the database 208, to determine likelihood of efficacy of Notch-targeted therapy in the subject. The susceptibility can be based on the single parameter (the level of VEGF-C expression), or can involve a calculation based on other genetic and non-genetic data, as described above, that is collected and included as part of the input 204 from the human subject, and that also is stored in the database 208 with respect to a population of other humans. Generally speaking, each parameter of interest is weighted to provide a conclusion with respect to the likelihood that Notch-targeted therapy would be effective. Such a conclusion is expressed in the conclusion in any statistically useful form, for example, as an odds ratio or a probability that the Notch-targeted therapy would be effective to treat the cancer.

In some variations, the system as just described further includes a communication tool 212. For example, the communication tool is operatively connected to the analysis routine 210 and comprises a routine stored on a computer-readable medium of the system and adapted to be executed on a processor of the system, to: generate a communication containing the conclusion; and to transmit the communication to the human subject 200 or the medical practitioner 202, and/or enable the subject or medical practitioner to access the communication. (The subject and medical practitioner are depicted in the schematic FIG. 17, but are not part of the system per se, though they may be considered users of the system. The communication tool 212 provides an interface for communicating to the subject, or to a medical practitioner for the subject (e.g., doctor, nurse, genetic counselor), the conclusion generated by the analysis tool 210 with respect to probability that the Notch-targeted therapy would be effective to treat the cancer in the subject. Usually, if the communication is obtained by or delivered to the medical practitioner 202, the medical practitioner will share the communication with the human subject 200 and/or counsel the human subject about the medical significance of the communication. In some variations, the communication is provided in a tangible form, such as a printed report or report stored on a computer readable medium such as a flash drive or optical disk. In some variations, the communication is provided electronically with an output that is visible on a video display or audio output (e.g., speaker). In some variations, the communication is transmitted to the subject or the medical practitioner, e.g., electronically or through the mail. In some variations, the system is designed to permit the subject or medical practitioner to access the communication, e.g., by telephone or computer. For instance, the system may include software residing on a memory and executed by a processor of a computer used by the human subject or the medical practitioner, with which the subject or practitioner can access the communication, preferably securely, over the internet or other network connection. In some variations of the system, this computer will be located remotely from other components of the system, e.g., at a location of the human subject's or medical practitioner's choosing.

In some variations, the system as described (including embodiments with or without the communication tool) further includes components that add a treatment or prophylaxis utility to the system. For instance, value is added to a determination of the likelihood that Notch-targeted therapy would be effective to treat the cancer when a medical practitioner can prescribe a Notch-targeted therapy and/or administer a standard of care to facilitate early treatment. Exemplary medicinal and surgical intervention protocols include administration of pharmaceutical agents for prophylaxis; and surgery, including in extreme cases surgery to remove a tissue or organ before the cancer within the organ metastasizes. Exemplary diagnostic protocols include non-invasive and invasive imaging; monitoring metabolic biomarkers; and biopsy screening.

For example, in some variations, the system further includes a medical protocol database 214 operatively connected to a computer-readable medium of the system and containing information correlating the level of VEGF-C expression and medical protocols for human subjects with the cancer. Such medical protocols include any variety of medicines, lifestyle changes, diagnostic tests, increased frequencies of diagnostic tests, and the like that are designed to achieve effective therapy with minimum side effects. The information correlating the level of VEGF-C expression with protocols could include, for example, information about the success of the Notch-targeted therapy, if a subject has a level of VEGF-C expression and follows a protocol.

By way of example, in some variations, a system that analyzes inputs of VEGF-C expression or assessing the therapeutic potential of the Notch-targeted therapy could generate a variety of treatment protocols, including for a subject with a cancer characterized by elevated VEGF-C expression, prescribing and/or administering a therapeutic regimen that includes Notch-targeted therapy; a therapeutic regimen that includes a molecule that suppresses expression or activity of Notch (e.g., a Notch inhibitor described herein).

The system of this embodiment further includes a medical protocol tool or routine 216, operatively connected to the medical protocol database 214 and to the analysis tool or routine 210. The medical protocol tool or routine 216 preferably is stored on a computer-readable medium of the system, and adapted to be executed on a processor of the system, to: (i) compare (or correlate) the conclusion that is obtained from the analysis routine 210 (with respect to metastatic potential of the cancer in the subject) and the medical protocol database 214, and (ii) generate a protocol report with respect to the probability that one or more medical protocols in the medical protocol database will achieve one or more of the goals of eliminating the cancer, slowing cancer growth or metastasis, and minimizing deleterious side effects of cancer treatment.

Some variations of the system just described include the communication tool 212. In some examples, the communication tool generates a communication that includes the protocol report in addition to, or instead of, the conclusion with respect to metastatic potential.

Information about the level of VEGF-C expression not only can provide useful information about identifying or quantifying the likelihood of therapeutic efficacy with Notch-targeted therapy; it can also provide useful information about possible causative factors for a human subject identified with a cancer, and useful information about therapies for the cancer patient. In some variations, systems of the invention are useful for these purposes.

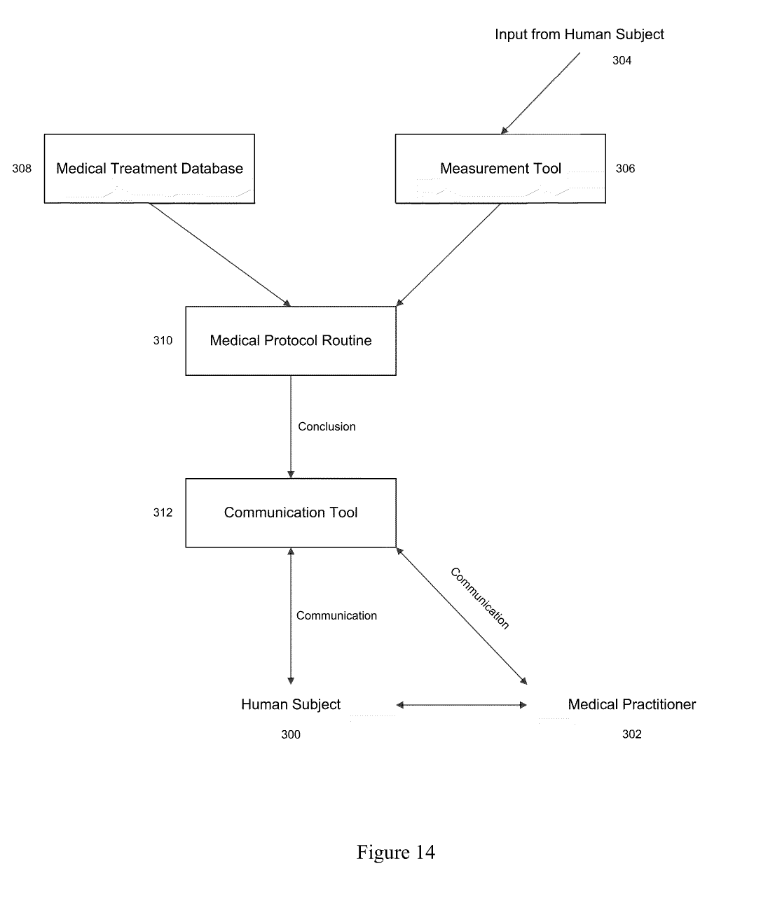

For instance, in some variations the invention is a system for assessing or selecting a treatment protocol for a subject diagnosed with a cancer. An exemplary system, schematically depicted in FIG. 14, comprises: (a) at least one processor; (b) at least one computer-readable medium; (c) a medical treatment database 308 operatively connected to a computer-readable medium of the system and containing information correlating the correlating the measurement of VEGF-C expression and efficacy of Notch-targeted therapy data in a population of humans with cancer; (d) a measurement tool 306 to receive an input (304, depicted in FIG. 14 but not part of the system per se) about the human subject and generate information from the input 304 about the correlating the measurement of VEGF-C expression and efficacy of Notch-targeted therapy data in a human subject diagnosed with the cancer; and (e) a medical protocol routine or tool 310 operatively coupled to the medical treatment database 308 and the measurement tool 306, stored on a computer-readable medium of the system, and adapted to be executed on a processor of the system, to compare the information with respect to the level of VEGF-C expression in a biological sample of the subject and the medical treatment database, and generate a conclusion with respect to at least one of (i) the probability that one or more medical treatments will be efficacious for treatment of the cancer for the patient; and (ii) which of two or more medical treatments for the cancer will be more efficacious for the patient.

Preferably, such a system further includes a communication tool 312 operatively connected to the medical protocol tool or routine 310 for communicating the conclusion to the subject 300, or to a medical practitioner for the subject 302 (both depicted in the schematic of FIG. 17, but not part of the system per se). An exemplary communication tool comprises a routine stored on a computer-readable medium of the system and adapted to be executed on a processor of the system, to generate a communication containing the conclusion; and transmit the communication to the subject or the medical practitioner, or enable the subject or medical practitioner to access the communication.

The invention may be more readily understood by reference to the following examples, which are given to illustrate the invention and not in any way to limit its scope.

### EXAMPLES

### Example 1

### VEGF-C as a Biomarker for Notch Inhibitor Sensitivity

The following Example demonstrates that tumors expressing an elevated level of VEGF-C sensitizes the tumor to treatment with a Notch inhibitor.

### Materials and Methods

Cell Line, Cell Culture and Viral Transduction:

SW480R(R18), a subline of SW480, is a human colon adenocarcinoma cell line as previously used and described (Petrova et al., 2008). NCI-H460-LNM35 (LNM35), a subline of LNM35-H460-N15, is a human lung cancer cell line as previously used and characterized (Kozaki et al., 2000). SK-MeI-103 is a human melanoma cell line (gifted by Dr Maria Soengas, Centro Nacional de Investigaciones Oncologicas, Madrid, Spain—obtained December 2010 and used in passages below 20). LNM35 cells were maintained in RPMI-1640 medium supplemented with 2 mM L-glutamine, penicillin (100 U/mL), streptomycin (100 μg/mL), and 10% fetal calf serum (FCS). SW480R and SK-MeI-103 cells were maintained in Dulbecco's Modified Eagles' Medium (DMEM) supplemented with 2 mM L-glutamine, penicillin (100 U/mL), streptomycin (100 μg/mL) and 10% FCS. The SW480R cell lines that stably express VEGF-C(SW-VEGF-C) or luciferase (SW-luc) were established by infection with a lentivirus encoding full length human VEGF-C or firefly luciferase (the viruses were generous gifts from Dr. Karin Joss, Cell Genesys, Calif., USA). The SK-MeI-103 cells which express VEGFR-3(D1-3)-Fc were established by retrovirus transfection. The VEGFR-3(D1-3)-Fc is a fusion protein of the immunoglobulin-like domains 1-3 of the extracellular part of human VEGFR-3 and the Fc part of human immunoglobulin G1 (Mäkinen at al., 2001). The VEGFR-3(D1-3)-Fc was amplified by PCR and cloned into HindIII-NotI site of the pMXs vector. The retrovirus was made as previously described (Zheng et al., 2011). The HDMECs, BECs and LECs were purchased from PromoCell, maintained on fibronectin-coated dishes in Endothelial Cell Basal Medium MV (EBCM; PromoCell) with growth supplements provided by the manufacturer and used in passages 2-6. VEGF-C (100 ng/mL) was added to the LEC growth medium (Karpanen et al., 2006).

Sprout-Like Structure in 2D Culture of LECs and BECs:

1:1 Mixtures of BECs and LECs were grown on cover slips for 48 hours without VEGF-C to allow them to make LECs islands surrounded by BECs. The cells were then incubated for a further 48 hours with 200 ng/ml VEGF-C (produced by Dr Michael Jeltsch, University of Helsinki) with or without 10 μg/ml recombinant human Dll4-Fc protein (R&D Systems). After fixation with 1% PFA, the samples were immunostained for PECAM-1, a blood vascular endothelial marker, and podoplanin, a lymphatic endothelial marker, to identify BECs and LECs. The number of sprout-like projections of BECs on LECs islands was then counted under a fluorescence microscope.

3D Spheroid Sprouting Assay:

BECs were cultured in round-bottom 96-well plates (ThermoScientific) precoated with 0.8% agarose for one day for spheroid formation. The spheroids were collected and embedded in 20% Matrigel-containing (BD Biosciences) fibrin gels (3 mg/ml fibrinogen, 2 U/ml thrombin and 200 μg/ml aprotinin), treated for 48 hours with hIgG (Sigma-Aldrich), Dll4-Fc, VEGF-C or their combinations in EBCM containing 1% FCS at final concentrations indicated in figure legends for 48 h. The spheroids were fixed in 4% PFA for 1 hour at room temperature.

AAV Vector Production:

The recombinant AAV serotype 9 (AAV9) was produced as described previously (Anisimov et al., 2009) with the modification that p5e18-VP2/9 serotype helper plasmid was used instead of the p5e18-VP2/8 plasmid. The AAV9 was packaged in 293T cells and the AAVs purified by ultracentrifugation with iodixanol gradient. The concentration of virus was quantified by real-time qPCR with primers specific for virus genome (5′-TTGTTGTTA ACTTGTTTATTGCAGC-3′ (SEQ ID NO: 23) and 5′-TGAGTTTGGACAAACCACAAC-3′ (SEQ ID NO: 24)). For the production of AAV9-Dll4-Fc, the Dll4-Fc coding sequence was cloned into psubCMV-WPRE vector for AAV9 production (FIG. 1A) (Zheng et al., 2011). Dll4-Fc is a fusion protein of mouse Dll4 ECD and human immunoglobulin G Fc region dimerized by the disulphide bond between the Fc domains (FIG. 1B).

Tumor Implantation:

Six- to eight-week old female nu/nu BALB/c mice were purchased from Harlan Laboratories (Venray, The Netherlands). The National Board for Animal Experiments at the Provincial State Office of Southern Finland approved all the experiments, which were performed in accordance with Finnish legislation regarding the humane care and use of laboratory animals. To induce the systemic overexpression of Dll4-Fc, or human serum albumin (HSA) as a negative control, the mice were anesthetized with ketamine (Ketalar, Pfizer) and xylaxine (Rompun vet, Bayer Healthcare) and the AAV9s encoding Dll4-Fc or HSA were injected intravenously at the dose of 1×10¹² vector genomes/mouse. 1 or 7 days after the injections, the tumor cells (5×10⁶) were implanted into the abdominal flank of the mice. Once the tumors became visible during the first few days, the size of the tumor was measured by a digital caliper every second day. The tumor volume was calculated using the formula: (length)×(width)×(depth)×0.52.

Antibodies, Immunocytochemistry, and Immunohistochemistry:

Cells were grown on coverslips, fixed in 1% PFA, permeabilized with 0.1% Triton X-100 and blocked with 5% donkey serum. The tumor tissues were fixed with 4% PFA, incubated in 25% sucrose, embedded into OCT compound (Sakura Finetek Europe), and cut into 10 μm sections. For immunostaining, the following primary antibodies were used:—rat anti-endomucin (Santa Cruz Biotechnology Inc.), anti-Lyve-1 antiserum (He et al., 2005), mouse anti-PECAM-1 (DAKO), rabbit anti-podoplanin (gift from Dr Kerjascki, Vienna, Austria), rabbit anti-cleaved Notch intracellular domain (Cell Signaling Technology Inc.), goat anti-human Prox-1 (R&D Systems) and rabbit anti-VEGF-C (serum #6; Saaristo et al., 2000). BEC spheroids were blocked in PBS containing 5% donkey serum, 0.2% BSA, 0.05% sodium azide and 0.3% Triton-X-100 for 30 min prior to incubation with mouse anti-human PECAM-1 antibody (DAKO) overnight. Alexa fluorochrome-conjugated secondary antibodies (Invitrogen) were used for signal detection.

BaF3/VEGFR-3 Assay:

The BaF3/VEGFR-3 cells are derived from the interleukin-3 (IL-3) dependent mouse pro-B cell line, BaF3 (Bernardi et al., 1987). The cells express a chimeric receptor consisting of the ECD of human VEGFR-3 fused in-frame to the transmembrane and intracellular domain of mouse erythropoietin receptor (Achen et al., 2000). In IL-3 deficient cell media, the cells can be rescued by the addition of VEGF-C. The serial dilutions of supernatants (50 μl each), as well as positive and negative controls were applied in the wells of 96-well plates in quadruplicate. Subsequently, 20,000 BaF3/VEGFR-3 cells in 50 μl were added and the cells incubated at 37° C. for 48 hours. MTT substrate was added and the cells incubated at 37° C. for 2 hours. Lysis buffer (10% SDS, 10 mM HCl) was added and the plates incubated at 37° C. overnight for colour development. Quantification was done by absorbance at 540 nm.

In Vitro Evaluation of Protein Expression by Transfected Cells:

To label proteins produced by 293T cells and cancer cell lines, the cells were starved for 30 min in methionine- and cysteine-free Eagle's Minimum Essential Medium, and metabolically labeled in this medium supplemented with EasyTag ³⁵S Protein Labeling Mix (Perkin Elmer Life Sciences) for 24 hours. Conditioned medium was then harvested and cleared of particulate materials by centrifugation. VEGF-C was precipitated with a human VEGFR-3(D1-3)-Fc protein (5 μg) and the complexes collected with protein A sepharose (Amersham Biosceinces and GE Healthcare). Dll4-Fc was directly precipitated with protein A sepharose (Amersham Biosciences). The precipitates were subjected to SDS-PAGE gel electrophoresis and visualized with autoradiography.

Dll4-Fc ELISA: To determine the Dll4-Fc concentration in serum at indicated time points after vector administration, a modified ELISA was carried out. Goat anti-mouse Dll4 antibody (R&D Systems) was coated on Maxisorp 96-well plates (Nunc) overnight at 4° C. The serum samples, taken from the tail vein, were applied in triplicate, and after washes, the horseradish peroxidase-conjugated anti-human immunoglobulin antibody (DAKO) was used as a detection antibody. The color was developed with SureBlue TMB Microwell Peroxidase Substrate (KPL). The reaction was stopped with an equal amount of 1M HCl and the optical density measured at 450 nm with a Multiscan Ascent plate reader.

Image Analysis:

The immunofluorescent images were captured with a Zeiss digital Axiocam camera connected to a Zeiss AxioPlan 2 microscope. Confocal images were captured using laser scanning confocal microscope Zeiss LSM 510 Meta. At least five tumors were examined in each experimental group, and images from at least three distinct areas characterized by the mean representative density of vessels were captured from each tumor under evaluation. The image quantification was carried out using ImageJ software (NIH, Bethesda, Md.).

Statistical Analysis:

Values are expressed as means±standard errors of the mean. Statistical analysis was performed with the unpaired t-test or with one-way analysis of variance (ANOVA) followed post-hoc analysis with Turkey's test or Games-Howell's test. PASWStatistics 18 (SPSS) software was used for the statistical analysis. Differences were considered statistically significantly at p<0.05. One asterisk (*) denotes for p<0.05 and two asterisks (**) for p<0.01.

### Results

VEGF-C Enhances Dll4-Fc-Induced Hypersprouting in BECs In Vitro:

Previous reports have indicated that Notch pathway inhibition induces excessive sprouting of blood vascular endothelial cells (Phng and Gerhardt, 2009). We evaluated if VEGF-C facilitates the Dll4-Fc-induced hypersprouting phenotype. To this end, an AAV serotype 9 vector encoding Dll4 ECD fused with the Fc part of the immunoglobulin G (AAV9-Dll4-Fc) was constructed. The expression construct for the Dll4-Fc is shown in FIG. 1A and FIG. 1B. The Dll4-Fc protein was secreted into the culture medium of AAV9-transduced 293T cells (FIG. 1C). For further in vitro assays, the Dll4-Fc protein was purified by protein A-sepharose affinity chromatography.

To determine if Dll4-Fc can efficiently bind to the endothelial cell surface, HDMECs, which represent a mixed population of BECs and LECs, were incubated with purified Dll4-Fc and stained for the Fc part. Co-staining for the LEC marker Prox-1 indicated that Dll4-Fc bound to both LECs and BECs (FIG. 1D). In the same experiment, Dll4-Fc decreased significantly the expression of the Notch downstream genes, Hes1, Hey2 and NRARP) (FIG. 1E), confirming the inhibitory potential of Dll4-Fc. To identify the cell type in which the Notch signal was inhibited, the cells for the cleaved/activated Notch intracellular domain (NICD) and Prox-1 were stained. The intensity of NICD was decreased in both LECs and BECs treated with Dll4-Fc or with the Notch cleavage (7-secretase) inhibitor, N—[N-(3,5-difluorophenacetyl)-1-alanyl]-S-phenyl-glycine t-butyl ester (DAPT) (FIG. 1F and FIG. 1G).

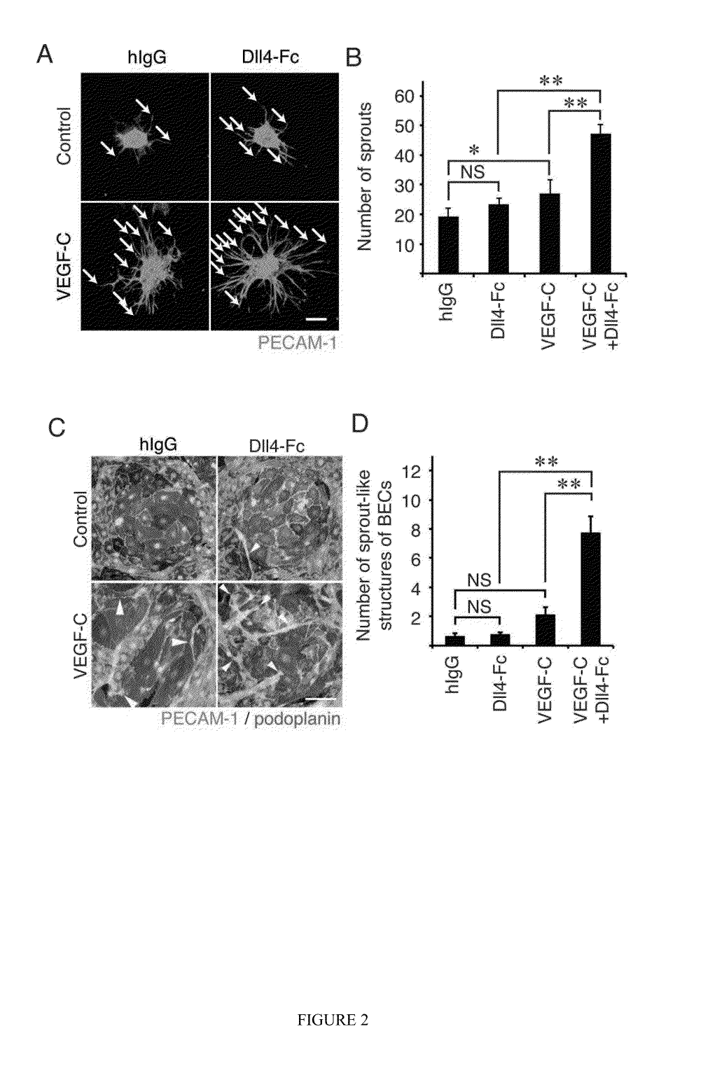

In order to evaluate the effects of Dll4-Fc on the BECs in vitro, a BEC spheroid sprouting assay was performed. The results indicated that a combination of Dll4-Fc and VEGF-C induced significantly more sprouts than Dll4-Fc or VEGF-C alone (FIG. 2A and FIG. 2B). In a mixed population of BECs and LECs in monolayer culture, homotypic cell adhesion produces islands of LECs surrounded by BECs. Supplementing these cultures with VEGF-C and/or Dll4-Fc induced sprout-like structure of BECs extending over the LECs island (FIG. 2C). VEGF-C tended to increase the sprout-like structure, but the difference was not statistically significant. However, a combination of VEGF-C and Dll4-Fc significantly increased the frequency of sprout-like structures (FIG. 2D). These results demonstrate that VEGF-C facilitates BEC sprouting induced by Dll4-Fc.

Dll4-Fc Strongly Suppresses the Growth of the SW480R Colon Carcinoma Tumors Overexpressing VEGF-C:

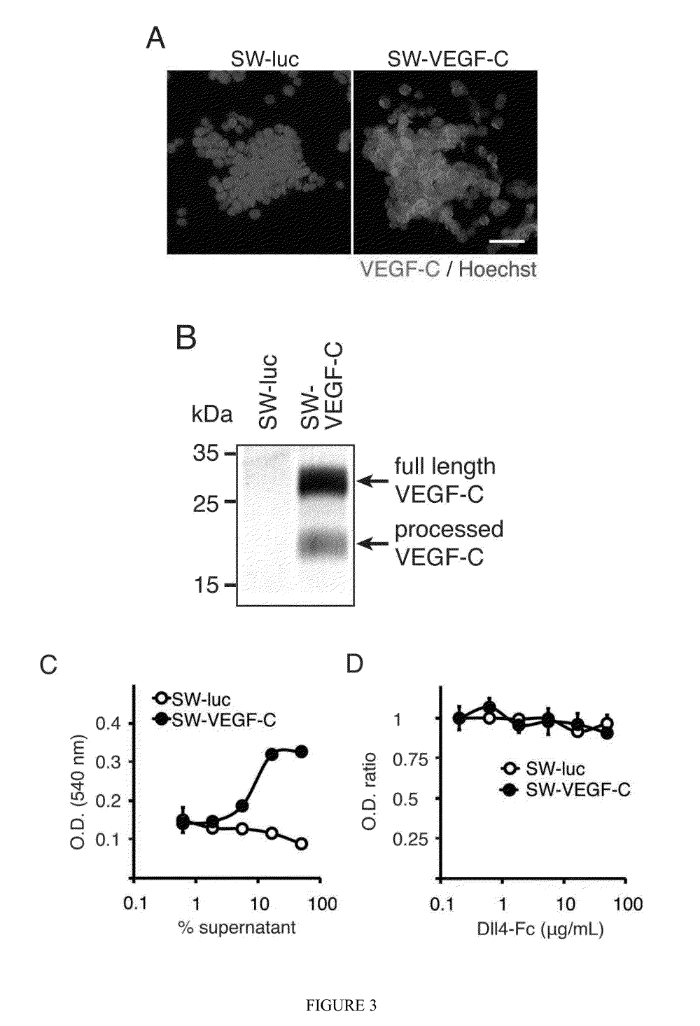

The effects of Dll4-Fc on the tumor vasculature in established tumor models were analyzed using tumor cell lines lentivirally transfected to overexpress VEGF-C. The SW480R human colon adenocarcinoma cell line was used which expresses trace amounts of VEGF-C and appears resistant to VEGFR-3 blocking antibody (Laakkonen et at., 2007). SW480R cells were transfected with lentiviruses to generate a VEGF-C overexpressing line (SW-VEGF-C) and a control cell line expressing luciferase (SW-Luc). The SW-Luc cells were confirmed to be negative for VEGF-C expression by immunofluorescent staining (FIG. 3A and FIG. 3B). In contrast, almost all of the SW-VEGF-C cells were positive for VEGF-C. The biological activity of VEGF-C secreted into the culture medium was quantified in vitro using the VEGF-C-dependent BaF3/VEGFR-3 survival/growth assay (FIG. 3C). The proliferation of SW480R cells was not affected by Dll4-Fc treatment even when used at high concentrations of up to 50 μg/ml (FIG. 3D), which suggests that the in vivo effects of Dll4-Fc cannot be explained by a direct anti-tumor effect of the transgenic protein.

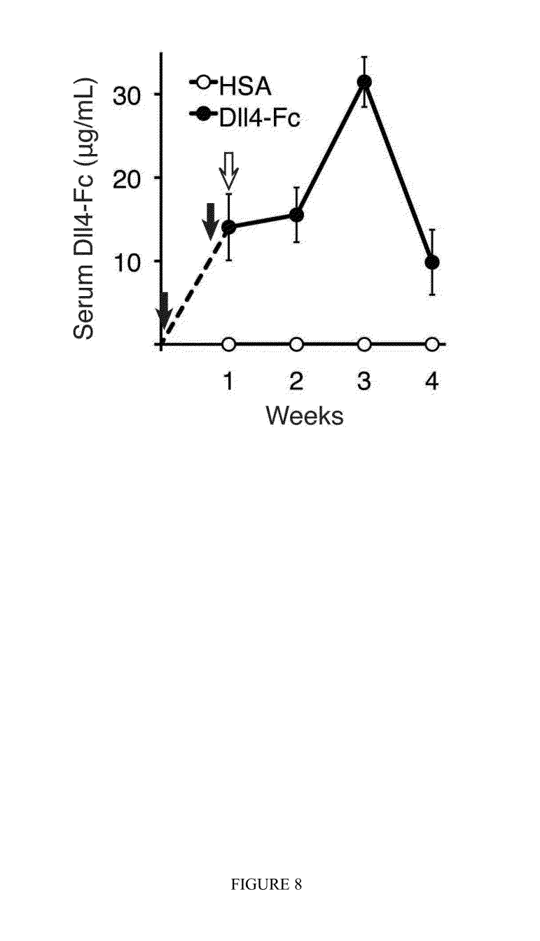

In order to test the effects of Dll4-Fc on tumor growth in vivo, the tumor cells were implanted into nu/nu mice treated by intravenous injection of AAV9-Dll4-Fc, or AAV9-HSA as a negative control, before tumor implantation. The serum concentration of Dll4-Fc was monitored by ELISA. The concentration was about 10 μg/ml one week after gene transduction, and it increased significantly after the second and third week (FIG. 8).

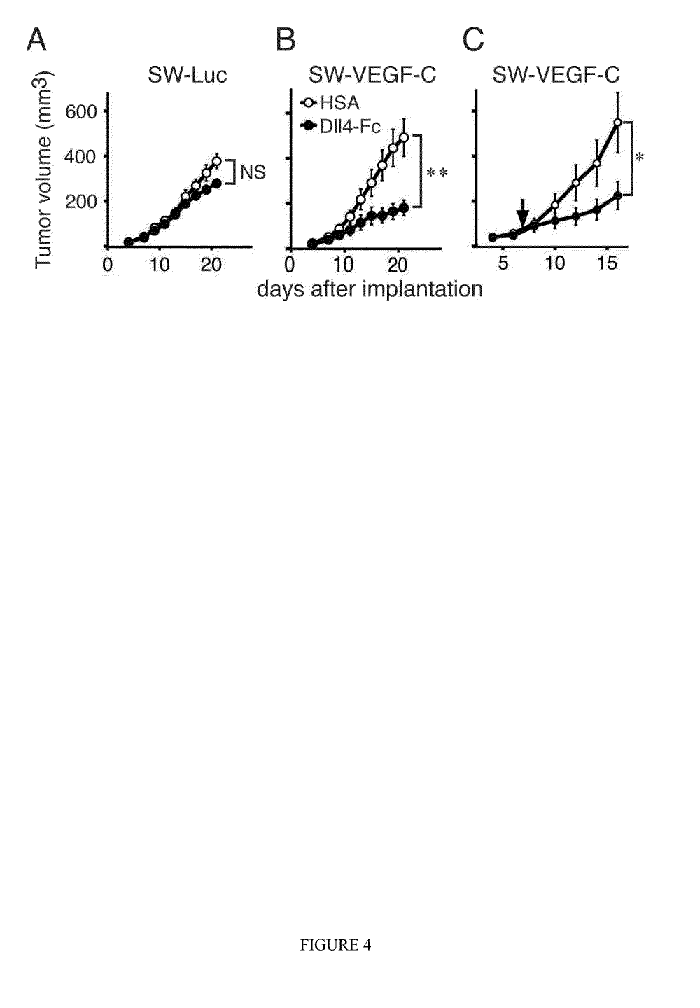

The AAV9-Dll4-Fc treatment resulted in a trend for inhibition of SW-Luc tumor growth (FIG. 4A). However, the extent of the tumor growth inhibition by the systemic Dll4-Fc therapy was significantly stronger in the tumors overexpressing VEGF-C (FIG. 4B). To study the interaction of Dll4-Fc and VEGF-C on tumor growth in a more clinically relevant setting, the virus was injected when the tumor growth was already evident 7 days after the tumor inoculation. A growth delay of VEGF-C overexpressing tumors was apparent starting 3 days after the AAV9-Dll4-Fc injection and a statistically significant difference was observed 16 days after implantation (FIG. 4C).

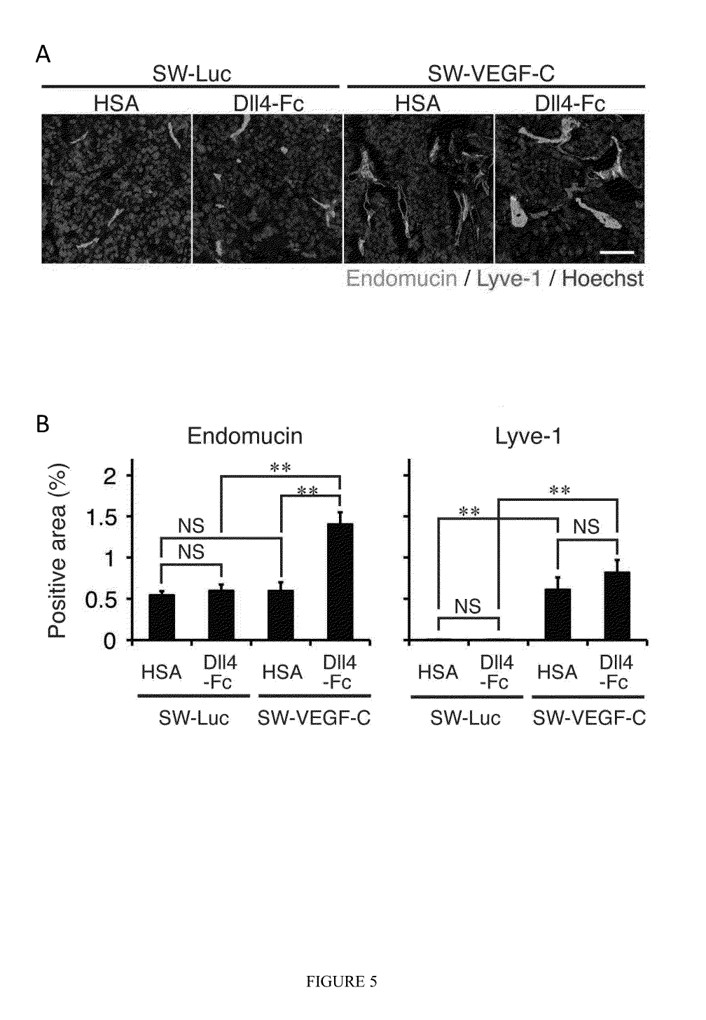

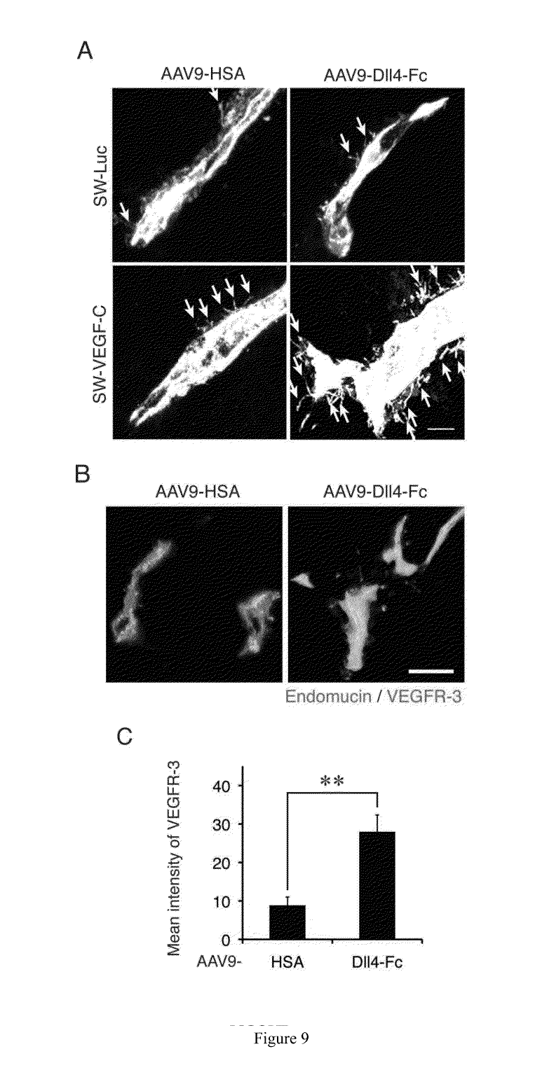

In order to elucidate the mechanism of growth suppression, tumor sections were stained for endomucin, a marker of blood vascular endothelial cells, and Lyve-1, a marker of lymphatic endothelial cells. In the SW-Luc tumors, the Dll4-Fc did not make any difference to the density of the blood and lymphatic vessels (FIG. 5A and FIG. 5B). In the SW-VEGF-C tumors, AAV9-Dll4-Fc increased blood vessel density when compared to AAV9-HSA, whereas lymphatic vessel density was not significantly affected. In contrast, no difference was found between the SW-Luc tumors growing in AAV9-HSA or AAV9-Dll4-Fc transfected mice (FIG. 5A and FIG. 5B). Analysis at high magnification indicated that the Dll4-Fc-treated SW-VEGF-C tumors contained more vessel sprouts than any tumors in other groups (FIG. 9). These results imply that the overexpression of VEGF-C sensitizes the SW480R tumor to the inhibitory effects of Dll4-Fc treatment by enhancing the non-productive endothelial sprouting.

Because it had been reported that tumor vessels were poorly covered with pericytes where Notch signaling was inhibited, the pericyte coverage was evaluated by α-SMA. There was no difference in the pericyte coverage between the tumors (data not shown). It had been reported that Prox-1 promotes malignant progression via disruption of cell polarity and adhesion in the SW480 cells (Skobe et al., 2001) and activation of Notch signaling down-regulates Prox-1 expression in the LECs (Laakkonen et al., 2007). Since it is possible that inhibition of Notch up-regulates Prox-1 in the SW480R cells and renders the cells more aggressive phenotype, we evaluated the expression of Prox-1 in the tumors. However, no difference in Prox-1 expression was found.

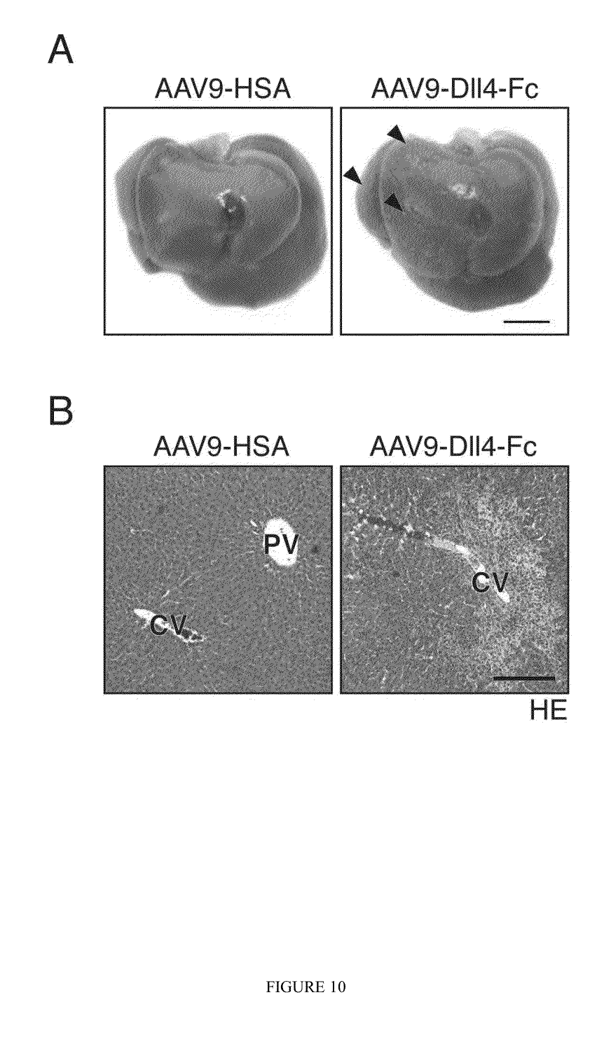

Yan (2010) reported that chronic Dll4 blockage induces histological changes in liver (centrilobular dilatation of sinusoids and atrophy of hepatocytes), elevation of liver enzymes, and tumors supposed to be vascular origin in the subcutaneous tissues, lungs and heart. In our study, dilation of centrilobular sinusoid and atrophy of the liver was observed (FIG. 10), whereas no tumors other than those from the implanted cells were found in any organs including the subcutaneous tissues, lungs and heart (data not shown).

Dll4-Fc Inhibits the Growth of Tumors that Express Endogenous VEGF-C:

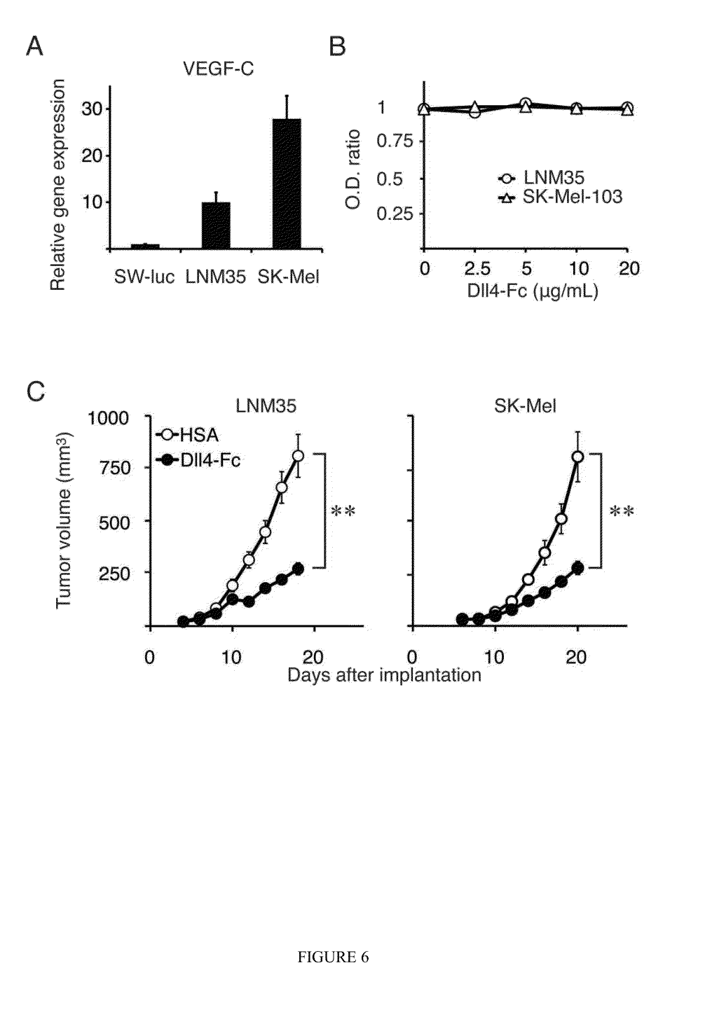

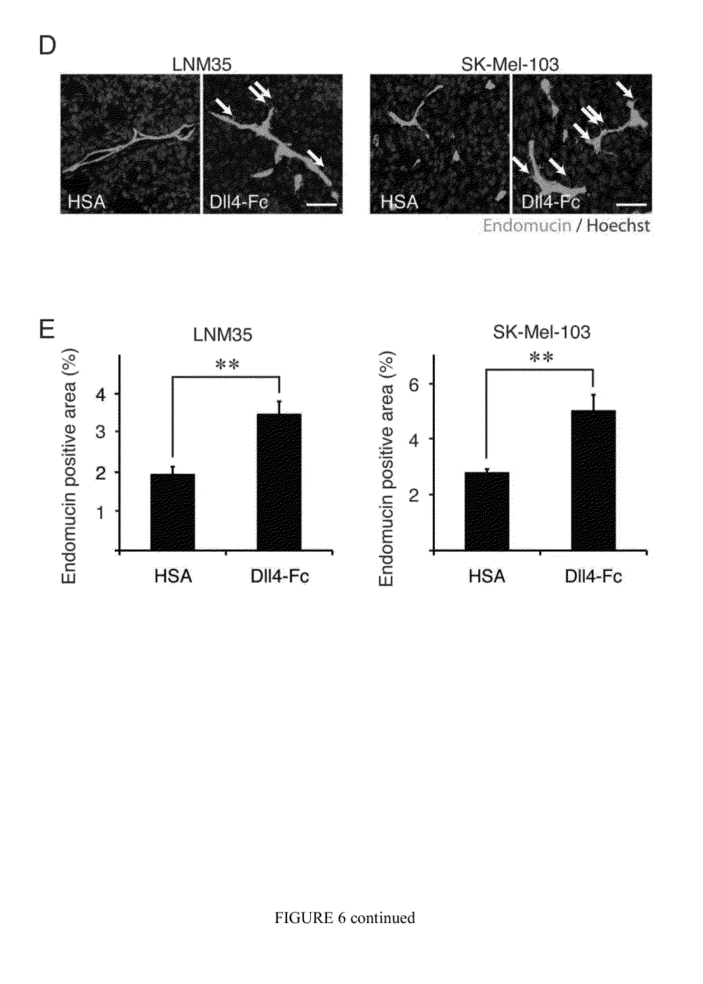

Tumors that express abundant endogenous VEGF-C were tested to see if they are responsive to Dll4-Fc. Analysis of steady-state mRNA levels indicated that LNM35 and SK-MeI-103 cells express 10 and 30 times more VEGF-C mRNA than SW-Luc cells, respectively (FIG. 6A). Although Dll4-Fc had no effect on the proliferation of these cells in vitro, the growth of tumor xenotransplants of LNM35 and SK-MeI-103 cells was strongly suppressed by Dll4-Fc (FIG. 6B and FIG. 6C). Tumor blood vessel sprouting and density were increased by Dll4-Fc in these tumors (FIG. 6D and FIG. 6E). Furthermore, Dll4-Fc increased the expression of VEGFR-3 in the tumor blood vessels, consistent with published data (Tammela et al., 2008) (FIG. 9). These results indicate that Dll4-Fc increases vessel sprouting in tumors, which express high levels of endogenous VEGF-C, and suppresses their growth in vivo.

Neutralization of VEGF-C/D Desensitizes Tumor Cells to Dll4-Fc:

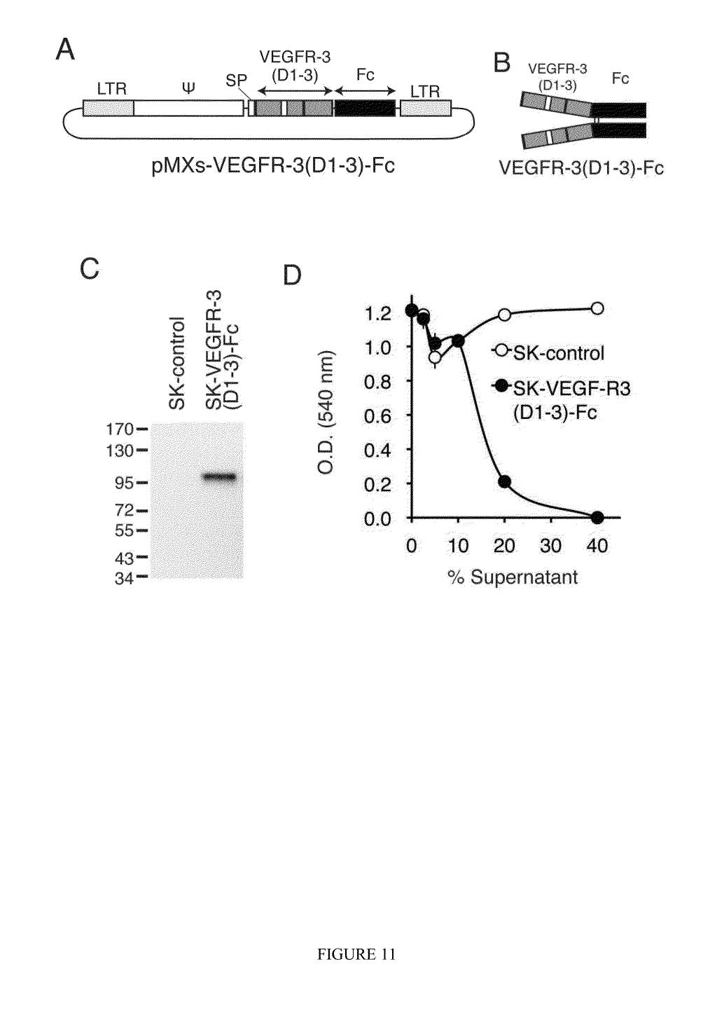

We tested if suppression of the VEGF-C/D-VEGFR-3 signaling desensitizes tumor cells to Dll4-Fc. SK-MeI-103 cells that express the extracellular domains 1-3 of VEGFR-3 fused to Fc part of human IgG (VEGFR-3(D1-3)-Fc) (FIG. 11A and FIG. 11B) were generated by retrovirus infection. VEGFR-3(D1-3)-Fc is a smaller molecule than VEGFR-3-Ig used in earlier studies (He et al., 2005) and can efficiently capture VEGF-C/D, thus serving as a VEGF-C/D trap (Mäkinen et al., 2001). It was confirmed that VEGFR-3(D1-3)-Fc produced by the retroviral vector suppresses BaF3/VEGFR-3 cell proliferation induced by VEGF-C (FIG. 5C and FIG. 5D).

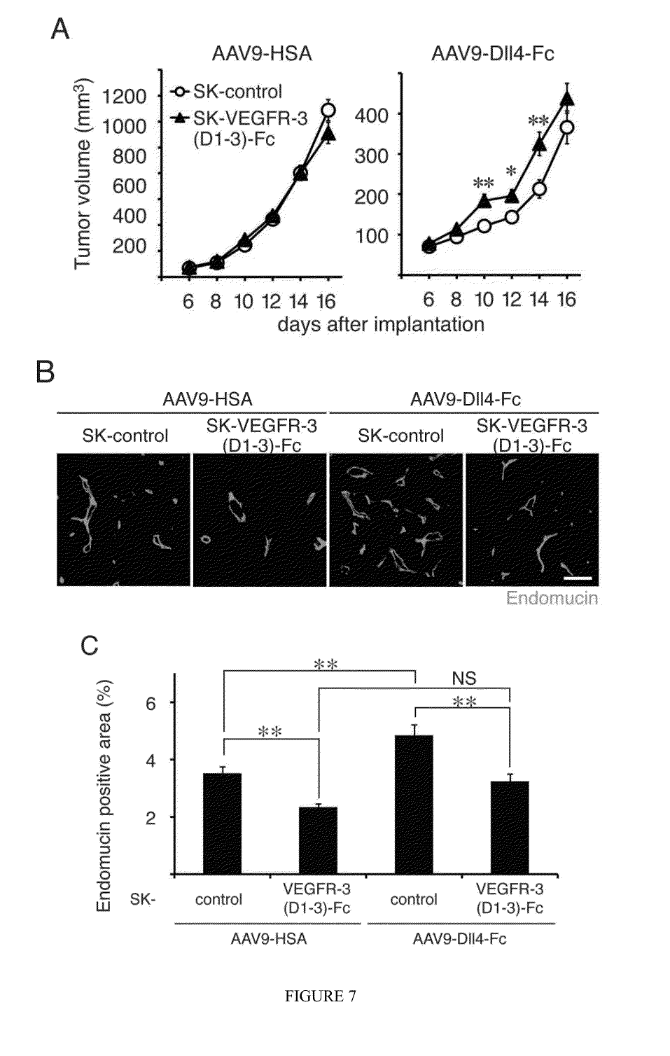

The SK-Mel-103 cells were transfected by an empty retrovirus (control) or by a retrovirus encoding VEGFR-3(D1-3)-Fc and then subcutaneously inoculated into nu/nu mice treated with the AAV9-HSA or the AAV9-Dll4-Fc as described above. In both control and VEGFR-3(D1-3)-Fc expressing tumor groups, Dll4-Fc suppressed tumor growth significantly. Furthermore, AAV9-Dll4-Fc provided stronger tumor growth inhibition in tumors expressing endogenous VEGF-C (transfected with empty retrovirus), than in VEGFR-3(D1-3)-Fc expressing tumors (FIG. 7A). This difference was statistically significant when measured 10, 12 and 14 days after tumor inoculation. The inhibition, however, was less than in VEGF-C overexpressing SW480R tumor, probably due to lower endogenous VEGF-C level in the SK-MEL-103 cells. No difference in growth rates was found between tumors transfected with HSA and the VEGFR-3(D1-3)-Fc or empty retrovirus vectors. Thus, the SK-MeI-103 tumors were not sensitive to growth inhibition by the VEGF-C/D trap. Instead, the neutralization of VEGF-C/D by the trap decreased growth suppression by Dll4-Fc, consistent with the results described above. Analysis of vascular density in the tumors indicated that the Notch inhibitor Dll4-Fc plus VEGF-C expression increased vessel density, while the VEGF-C/D trap downregulated endothelial proliferation (consistent with published data).

### Discussion

Inhibition of Notch signaling has become a mechanism by which cancer is targeted. Notch inhibition induces disorganized, poorly-perfused blood vessels were induced by Notch inhibition, which result in suppression of tumor growth. But, it is not well known what kind of tumors are sensitive to the therapy. In this study, we have shown that VEGF-C overexpressing tumors are more sensitive than non-overexpressing tumors in the mouse xenograft model. Overexpression of VEGF-C in tumors can enhance the non-productive angiogenesis induced by blocking Dll4-Notch signaling, thus resulting in a significant additive inhibition of tumor growth. Consistent results were obtained using tumor cell lines expressing high endogenous VEGF-C levels, which was blocked by tumor specific expression of soluble VEGF-C trap.

Our results implicate VEGF-C as a useful marker to select patients for Notch inhibition therapy.

When considering an indication of cancer chemotherapy, it would be nice to know to which chemotherapeutic drugs a cancer is sensitive. Scehnet et al. (2007) showed that HM7, Colo205, and Calu6 are very sensitive to anti-Dll4 antibody, but MV-522 human lung adenocarcinoma cell line is less sensitive than these cell lines. As shown in our study, in xenograft implantation model, the SW-luc cell line was not sensitive to AAV9-induced Dll4-Fc. However, SW-VEGF-C was very sensitive to Dll4-Fc. The results indicate that tumors overexpressing VEGF-C are much more sensitive to the Notch-targeting therapy. the results further indicate that clinicians can select a patient suitable for Notch-targeting therapy by checking VEGF-C expression in a biopsy or surgical specimen before the chemotherapy starts.

The main target of anti-Notch signal therapies is thought to be blood vessels induced by a tumor, rather than tumor cells themselves. In our experiments, Dll4-Fc did not have any effects on the proliferation of tumor cells in vitro, but Dll4-Fc did have the effects in vivo. In the SW-luc cells, Dll4-Fc had no effects on the blood vessels, however the Dll4-Fc increased blood vessels in the SW-VEGF-C cells. The results suggest that a tumor with low concentration of VEGF-C is not sensitive to the Notch inhibition by Dll4-Fc, but a tumor with high VEGF-C is sensitive to the inhibition. The VEGF-C induces both blood vessels and lymphatic vessels in the tumor. The Dll4-Fc had no effect on the lymphatics, but did have an effect on the blood vessels. The increase of blood vessels induced by Notch inhibition is reported to be accompanied by compromised perfusion (Noguera-Troise et al., 2006; Ridgway et al., 2006). The main reason for the effectiveness of the Dll4-Fc on the SW-VEGF-C is thought to be the effect on the blood vessels. Tammela et al. (2008) reported that inhibition of Notch upregulates VEGFR-3 in the endothelial cells and makes them sensitive to VEGF-C, and the signal from VEGF-C is important for angiogenesis. Our results support their data.

Hoey et al. (2009) reported that inhibition of Notch signaling reduced the population of cancer stem cells. Pretreatment with Dll4-Fc might reduce the cancer stem cell in the SW480R cells and affect the effectiveness of tumor inoculation. However, in our study, the AAV9-Dll4-Fc was also effective on the SW-VEGF-C cells even when it was injected after the tumors were implanted (FIG. 4C). This result indicates that the effects of Dll4-Fc in this setting mainly affect the development of the tumor vasculature and the Notch-targeting therapy is effective even after the tumor was detected and the expression of VEGF-C was evaluated by immunohistochemistry from the biopsy or the surgically excised samples.

The therapies targeting Notch signaling seem to be promising, but which tumors are sensitive to the therapies are not known. Our results identify VEGF-C as a useful marker to predict sensitivity of a cancer to anti-Notch signaling therapies.

The foregoing examples are intended to be illustrative of the invention and not intended to limit the claims which define the invention. All patent, journal, and other literature cited herein is incorporated herein by reference in their entireties.

### REFERENCES

Achen M. G., Roufail S., Domagala T., et al. (2000). Monoclonal antibodies to vascular endothelial growth factor-D block its interactions with both VEGF receptor-2 and VEGF receptor-3. Eur. J. Biochem., 267(9), 2505-2515.
Achen M. G., McColl B. K. & Stacker S. A. (2005). Focus on lymphangiogenesis in tumor metastasis. Cancer Cell; 7(2), 121-127.
Alitalo K. (2011). Fifteen years of molecular lymphangiogenesis—an interview with Kari Alitalo by Tatiana Petrova. Int. J. Dev. Biol., 55(4-5), 389-394.
Anisimov A., Alitalo K., Korpisalo P., et al. (2009). Activated forms of VEGF-C and VEGF-D provide improved vascular function in skeletal muscle. Circ Res., 104(11), 1302-1312.
Artavanis-Tsakonas S., Rand M. D. & Lake RJ. (1999). Notch signaling: Cell fate control and signal integration in development. Science, 284(5415), 770-776.
Beals J. K. (2007). Reporting on press briefing by Dr. Siemers at Alzheimer's Association International Conference on Prevention of Dementia: Abstract HT-005. Presented Jun. 11, 2007-Medscape Medical News.
Benedito R., Rocha S. F., Woeste M., et al. (2012). Notch-dependent VEGFR3 upregulation allows angiogenesis without VEGF-VEGFR2 signalling. Nature, 484(7392), 110-114.
Bernardi P., Patel V. P., Lodish H. F. (1987). Lymphoid precursor cells adhere to two different sites on fibronectin. J. Cell Biol., 105(1), 489-498.
BrouC., Logeat F., Gupta N., Bessia C., LeBail 0., Doedens J. R., Cumano A., Roux P., Black R. A. & Israel A. (2000). A novel proteolytic cleavage involved in Notch signaling: the role of the disintegrin-metalloprotease TACE. Mol. Cell, 5(2), 207-216.
Brummelkamp T. R., Bernards R. & Agami R. (2002). A system for stable expression of short interfering RNAs in mammalian cells. Science, 296(5567), 550-553.
Bullock, G. R. et al., Techniques in Immunocytochemistry, Academic Press, Orlando, Fla. Vol. 1 (1982), Vol. 2 (1983), Vol. 3 (1985)
Chard, T. (1986). An Introduction to Radioimmunoassay and Related Techniques, Elsevier Science Publishers, Amsterdam, The Netherlands
Choo Y., Castellanos A., Garcia-Hernandez B., Sanchez-Garcia I. & Klug A. (1997). Promoter-specific activation of gene expression directed by bacteriophage-selected zinc fingers. J. Mol. Biol., 273(3), 525-532.
Chowrira B. M. & Burke J. M. (1992). Extensive phosphorothioate substitution yields highly active and nuclease-resistant hairpin ribozymes. Nucleic Acids Res., 20(11), 2835-2840.
Cole S. P. C., Kozbor D. & Roder, J. C. (1985). The EBV-hybridoma technique and its application to human lung cancer. In, Monoclonal Antibodies and Cancer Therapy. (eds. R. A. Reisfeld and S. Sell), Alan R Liss Inc, New York N.Y., pp 77-96.
Cote R. J., Morrissey D. M., Houghton A. N., Beattie E. J. Jr, Oettgen H. F. & Old L. J. (1983). Generation of human monoclonal antibodies reactive with cellular antigens. Proc. Natl. Acad. Sci. USA., 80(7), 2026-2030
Crooke S. & Lebleu, B. eds. (1983). Antisense Research and Applications, CRC Press, Boca Raton, Fla.
Daubendiek S. L. & Kool E. T. (1997). Generation of catalytic RNAs by rolling transcription of synthetic DNA nanocircles. Nat. Biotechnol., 15(3), 273-277.
De Strooper B., Annaert W., Cupers P., Saftig P., Craessaerts K., Mumm J. S., Schroeter E. H., Schrijvers V., Wolfe M. S., Ray W. J., Goate A. & Kopan R. (1999). A presenilin-1-dependent gamma-secretase-like protease mediates release of Notch intracellular domain. Nature, 398(6727), 518-522.
Elbashir S. M., Martinez J., Patkaniowska A., Lendeckel W. & Tuschl T. (2001). Functional anatomy of siRNAs for mediating efficient RNAi in Drosophila melanogaster embryo lysate. EMBO J., 20(23), 6877-6888.
Elbashir S. M., Harborth J., Lendeckel W., Yalcin A., Weber K. & Tuschl T. (2001). Duplexes of 21-nucleotide RNAs mediate RNA interference in cultured mammalian cells. Nature, 411(6836). 494-498.
Elbashir S. M., Lendenckel I. W. & Tuschl T. (2011). RNA interference is mediated by 21- and 22-nucleotide RNAs. Genes Dev., 15(2), 188-200.
Fingl E. & Woodbury D. M. (1975). Chatper 1 in “The Pharmacological Basis of Therapeutics”, eds. Goodman L. S, and Gilmam A. 5^{th} Edition. NY MacMillan.
Fishwild D. M., O'Donnell S. L., Bengoechea T., Hudson D. V., Harding F., Bernhard S. L., Jones D., Kay R. M., Higgins K. M., Schramm S. R. & Lonberg N. (1996). High-avidity human IgG kappa monoclonal antibodies from a novel strain of minilocus transgenic mice. Nature Biotechnology, 14(7), 845-851.
Fire A., Xu S., Montgomery M. K., Kostas S. A., Driver S. E. & Mello C. C. (1998). Potent and specific genetic interference by double-stranded RNA in Caenorhabditis elegans. Nature, 391(6669), 806-811.
Funahashi Y., Hernandez S. L., Das I., Ahn A., Huang J., Vorontchikhina M., Sharma A., Kanamaru E., Borisenko V., Desilva D. M., Suzuki A., Wang X., Shawber C. J., Kandel J. J., Yamashiro D. J. & Kitajewski J. (2008). A notchl ectodomain construct inhibits endothelial notch signaling, tumor growth, and angiogenesis. Cancer Res., 68(12), 4727-4735.
Gnatenko D., Arnold T. E., Zolotukhin S., Nuovo G. J., Muzyczka N. & Bahou W. F. (1997). Characterization of recombinant adeno-associated virus-2 as a vehicle for gene delivery and expression into vascular cells. J. Invest. Med., 45(2), 87-98.
Gordon W. R., Vardar-Ulu D., Histen G., Sanchez-Irizarry C., Aster J. C. & Blacklow S. C. (2007). Structural basis for autoinhibition of Notch. Nat. Struct. Mol. Biol., 14(4), 295-300.
Greisman H. A. & Pabo C. O. (1997). A general strategy for selecting high-affinity zinc finger proteins for diverse DNA target sites. Science, 275(5300), 657-661.
Gupta-Rossi N., Six E., LeBail 0., Logeat F., Chastagner P., Olry A., Israel A. & Brou C. (2004). Monoubiquitination and endocytosis direct gamma-secretase cleavage of activated Notch receptor. J. Cell Biol., 166(1), 73-83
Harlow E. & Lane D. (1988). Antibodies: A Laboratory manual. Cold Spring Harbor Laboratory, Cold Spring Harbor, N.Y.
He Y., Kozaki K., Karpanen T., et al. (2002). Suppression of tumor lymphangiogenesis and lymph node metastasis by blocking vascular endothelial growth factor receptor 3 signaling. Natl Cancer Inst., 94(11), 819-825.
He Y., Rajantie I., Pajusola K., et al. (2005). Vascular endothelial cell growth factor receptor 3-mediated activation of lymphatic endothelium is crucial for tumor cell entry and spread via lymphatic vessels. Cancer Res., 65(11), 4739-4746.
Helene C. (1991). The anti-gene strategy: control of gene expression by triplex-forming-oligonucleotides. Anticancer Drug Des., 6(6), 569-584.
Helene C., Thuong N. T. & Harel-Bellan A. (1992). Control of gene expression by triple helix-forming oligonucleotides. The antigene strategy. Ann, N.Y. Acad. Sci., 660, 27-36.
Hoey T., Yen W., Axelrod F., et al. (2009). DLL4 blockade inhibits tumor growth and reduces tumor-initiating cell frequency. Cell Stem Cell, 5(2), 168-77.
Hoogenboom H. R. & Winter G. (1992). By-passing immunisation. Human antibodies from synthetic repertoires of germline VH gene segments rearranged in vitro. J. Mol. Biol., 227(2), 381-388.
Hutvagner G. & Zamore P. D. (2002). A microRNA in a multiple-turnover RNAi enzyme complex. Science, 297(5589), 2056-2060.
Ilagan M. X. & Kopan R. (2007). SnapShot: notch signaling pathway, Cell 128(6), 1246.
Jarriault S., Brou C., Logeat F., Schroeter E. H., Kopan R & Israel A. (1995). Signalling downstream of activated mammalian Notch. Nature, 377, 355-358.
Jones L. J., Yue S. T., Cheung C. Y. & Singer V. L. (1998). RNA quantitation by fluorescence-based solution assay: RiboGreen reagent characterization. Anal. Biochem., 265(2), 368-374.
Kadambi A., Mouta Carreira C., Yun C. O., et al. (2001). Vascular endothelial growth factor (VEGF)-C differentially affects tumor vascular function and leukocyte recruitment: role of VEGF-receptor 2 and host VEGF-A. Cancer Res., 61(6), 2404-2408.
Kang J., Yoo J., Lee S., et al. (2010). An exquisite cross-control mechanism among endothelial cell fate regulators directs the plasticity and heterogeneity of lymphatic endothelial cells. Blood, 1-30.
Karpanen T., Egeblad M., Karkkainen M. J., et al. (2001). Vascular endothelial growth factor C promotes tumor lymphangiogenesis and intralymphatic tumor growth. Cancer Res., 61(5), 1786-1790.
Karpanen T., Heckman C. A., Keskitalo S., et al. (2006). Functional interaction of VEGF-C and VEGF-D with neuropilin receptors. FASEB J., 20(9), 1462-1472.
Kawasake H. & Taira K. (2003). He Hes1 is a target of microRNA-23 during retinoic-acid-induced neuronal differentiation of NT2 cells. Nature, 423(6942), 838-842.
Kim J. S., Kim J., Cepek K. L., Sharp P. A. & Pabo C. O. (1997). Design of TATA box-binding protein/zinc finger fusions for targeted regulation of gene expression. Proc. Natl. Acad. Sci. USA, 94(8), 3616-3620.
Kim V. N., Mitrophanous K., Kingsman S. M. & Kingsman A. J. (1998). Minimal requirement for a lentivirus vector based on human immunodeficiency virus type 1. J. Virol., 72(1), 811-816.
Kingsman & Johnson, Scrip Magazine, October, 1998, 43 46.
Koehler G. & Milstein C. (1975). Continuous cultures of fused cells secreting antibody of predefined specificity. Nature, 256(5517), 495-497.
Koizumi M, Hayase Y., Iwai S., Kamiya H., Inoue H. & Ohtsuka E. (1989). Design of RNA enzymes distinguishing a single base mutation in RNA. Nucleic Acid Res., 17(17), 7059-7071.
Kopan R. & Ilagan M. X. (2004). Gamma-secretase: proteasome of the membrane? Nat. Rev. Mol. Cell. Biol., 5(6), 499-504.
Kosbor et al., (1983). The production of monoclonal antibodies from human lymphocytes. Immunol. Today, 4, 72-79.
Kozaki K., Miyaishi O., Tsukamoto T., et al. (2000). Establishment and characterization of a human lung cancer cell line NCI-H460-LNM35 with consistent lymphogenous metastasis via both subcutaneous and orthotopic propagation. Cancer Res., 60(9), 2535-2540.
Kurreck J., Wyszko E., Gillen C. & Erdmann V. A. (2002). Design of antisense oligonucleotides stabilized by locked nucleic acids. Nucleic Acids Res., 30(9), 1911-1918.
Laakkonen P., Waltari M., Holopainen T., et al. (2007). Vascular endothelial growth factor receptor 3 is involved in tumor angiogenesis and growth. Cancer Res., 67(2), 593-9.
Lee N. S., Dohjima T., Bauer G., Li H., Li M. J., Ehsani A., Salvaterra P. & Rossi J. (2002). Expression of small interfering RNAs targeted against HIV-1 rev transcripts in human cells. Nat. Biotechnol., 20(5), 500-505.
Lewis C. E., De Palma M. & Naldini L. (2007). Tie2-expressing monocytes and tumor angiogenesis: regulation by hypoxia and angiopoietin-2. Cancer Res., 67(18), 8429-8432.
Li J., Sainson R. C. A., Shi W, et al. (2007). Delta-like 4 notch ligand regulates tumor angiogenesis, improves tumor vascular function, and promotes tumor growth in vivo. Cancer Res, 67(23), 11244-53.
Li K., Li Y., Wu W., Gordon W R., Chang D W., Lu M., Scoggin S., Fu T., Vien L., Histen G., Zheng J., Martin-Hollister R., Duensing T., Singh S., Blacklow S. C., Yao Z., Aster J. C. & Zhou B. B. (2008). Modulation of Notch signaling by antibodies specific for the extracellular negative regulatory region of NOTCH3. J. Biol. Chem., 283(12), 8046-8054.
Lima W. F., Brown-Driver V., Fox M., Hanecak R. & Bruice T. W. (1997). Combinatorial screening and rational optimization for hybridization to folded hepatitis C virus RNA of oligonucleotides with biological antisense activity. J. Biol. Chem., 272(1), 626-638.
Liu Q., Segal D. J., Ghiara J. B. & Barbas C. F. (1997). Design of polydactyl zinc-finger proteins for unique addressing within complex genomes. Proc. Natl. Acad. Sci. USA, 94(11), 5525-5530.
Lohela M., Helotera H., Haiko P., Dumont D. J. & Alitalo K. (2008). Transgenic induction of vascular endothelial growth factor-C is strongly angiogenic in mouse embryos but leads to persistent lymphatic hyperplasia in adult tissues. Am. J. Pathol., 173(6), 1891-1901.
Lonberg N., Taylor L. D., Harding F. A., Trounstine M., Higgins K. M., Schramm S. R., Kuo C. C., Mashayekh R., Wymore K., McCabe J. G., et al. (1994). Antigen-specific human antibodies from mice comprising four distinct genetic modifications. Nature, 368(6474), 856-859.
Lonberg N. & Huszar D. (1995). Human antibodies from transgenic mice. Intern. Rev. Immunol., 13(1), 65-93.
Long D. M. & Uhlenbeck O. C. (1993). Self-cleaving catalytic RNA. FASEB J., 7(1), 25-30.
Mäkinen T., Veikkola T., Mustjoki S., et al. (2001). Isolated lymphatic endothelial cells transduce growth, survival and migratory signals via the VEGF-C/D receptor VEGFR-3. EMBO J., 20(17), 4762-4773.
Mäkinen T., Jussila L., Veikkola T., et al. (2001). Inhibition of lymphangiogenesis with resulting lymphedema in transgenic mice expressing soluble VEGF receptor-3. Nature Medicine, 7(2), 199-205.
Marks J. D., Hoogenboom H. R., Bonnert T. P., McCafferty J., Griffiths A. D. & Winter G. (1991). By-passing immunization. Human antibodies from V-gene libraries displayed on phage. J. Mol. Biol., 222(3), 581-597.
Marks J. D., Griffiths A. D., Malmqvist M., Clackson T. P., Bye J. M. & Winter G. (1992). By-passing immunization: building high affinity human antibodies by chain shuffling. BioTechnology, 10(7), 779-783.
Maher, L. J. (1992). DNA triple-helix formation: an approach to artificial gene repressors? Bioassays, 14(12), 807-815.
Martinez J., Patkaniowska A., Urlaub H., Liihrmann R. & Tuschl T. (2002). Single-stranded antisense siRNAs guide target RNA cleavage in RNAi. Cell, 110(5), 563-574.
Mattila MM-T., Ruohola J. K., Karpanen T., Jackson D. G., Alitalo K. & Harkonen P. L. (2002). VEGF-C induced lymphangiogenesis is associated with lymph node metastasis in orthotopic MCF-7 tumors. Int. J. Cancer, 98(6), 946-951.
McColl D. J., Honchell C. D. & Frankel A. D. (1999). Structure-based design of an RNA-binding zinc finger. Proc. Natl. Acad. Sci. USA, 96(17), 9521-9526.
Melton D. A. ed. (1988). Antisense RNA and DNA (1988) Cold Spring Harbor Laboratory Cold Spring Harbor, N.Y.
Miele L. (2006). Notch signaling. Clin. Cancer Res., 12(4), 1074-1079.
Morrison S. L., Johnson M. J., Herzenberg L. A. & Oi V. T. (1984). Chimeric human antibody molecules: mouse antigen-binding domains with human constant region domains. Proc. Natl. Acad. Sci. USA, 81(21), 6851-6855.
Morrison S. L. (1994). Immunology. Success in specification. Nature, 368(6474), 812-813.
Moss M. L., Stoeck A., Yan W. & Dempsey P. J. (2008). ADAM10 as a target for anti-cancer therapy. Curr Pharm Biotechnol., 9(1), 2-8.
Nam Y., Sliz P., Pear W. S., Aster J. C. & Blacklow S. C. (2007). Cooperative assembly of higher-order Notch complexes functions as a switch to induce transcription. Proc. Natl. Acad. Sci. USA, 104(7), 2103-2108.
Nam Y., Sliz P., Song L., Aster J. C. & Blacklow S. C. (2006). Structural basis for cooperativity in recruitment of MAML coactivators to Notch transcription complexes. Cell, 124(5), 973-983
Neuberger M. S., Williams G. T. & Fox R. O. (1984). Recombinant antibodies possessing novel effector functions. Nature 312(5995), 604-608
Neuberger M. (1996). Generating high-avidity human Mabs in mice. Nature Biotechnology, 14(7), 826
Nickoloff B. J., Qin J. Z., Chaturvedi V., Denning M. F., Bonish B. & Miele L. (2002). Jagged-1 mediated activation of notch signaling induces complete maturation of human keratinocytes through NF-kappaB and PPARgamma. Cell Death Differ., 9(8), 842-855.
Noguera-Troise I., Daly C., Papadopoulos N. J., et al. (2006). Blockade of Dll4 inhibits tumour growth by promoting non-productive angiogenesis. Nature, 444(7122), 1032-1037.
Nykanen A., Haley B. & Zamore P. D. (2001). ATP requirements and small interfering RNA structure in the RNA interference pathway. Cell, 107(3), 309-321.
Okajima T. & Irvine K. D. (2002). Regulation of notch signaling by O-linked fucose. Cell, 111(6), 893-904.
Ojwang J. O., Hampel A., Looney D. J., Wong-Staal F. & Rappaport J. (1992). Inhibition of human immunodeficiency virus type 1 expression by a hairpin ribozyme. Proc. Natl. Acad. Sci. (USA), 89(22), 10802-10806.
Orlandi et al (Proc Natl Acad Sci 86: 3833-3837; 1989), and Winter G and Milstein C (Nature 349: 293-299, 1991).
Perrotta A. T. & Been M. D. (1992). Cleavage of oligoribonucleotides by a ribozyme derived from the hepatitis delta virus RNA sequence. Biochem., 31(1), 16-21.
Petrova T. V., Nykanen A., Norrmen C., et al. (2008). Transcription factor PROX1 induces colon cancer progression by promoting the transition from benign to highly dysplastic phenotype. Cancer Cell, 13(5), 407-419.
Phng L. & Gerhardt H. (2009). Angiogenesis: A team effort coordinated by notch. Developmental Cell, 16(2), 196-208.
Pitsouli C. & Delidakis C. (2005). The interplay between DSL proteins and ubiquitin ligases in Notch signaling. Development, 132(18), 4041-4050.
Quantin B., Perricaudet L. D., Tajbakhsh S. & Mandel J. L. (1992). Adenovirus as an expression vector in muscle cells in vivo. Proc. Natl. Acad. Sci. USA, 89(7), 2581-2584.
Ridgway J., Zhang G., Wu Y., et al. (2006). Inhibition of Dll4 signalling inhibits tumour growth by deregulating angiogenesis. Nature, 444(7122), 1083-1087.
Rizzo P., Osipo C., Foreman K., Golde T., Osborne B. & Meile L. (2008). Rational targeting of Notch signaling in cancer. Oncogene, 27(38), 5124-5131.
Rosen L. B., Stone J. A., Plump A., Yuan J., Harrison T., Flynn M., Dallob, A., Matthews C., Stevenson D., Schmidt D., Palmieri T., Leibowitz M., Jhee S., Ereshefsky L., Salomon R., Winchell G., Shearman M. S., Murphy M. G. & Gottesdiener K. M. (2006). The gamma secretase inhibitor MK-0752 acutely and significantly reduces CSF Abeta40 concentrations in human. Alzheimer's & Dementia, The Journal of the Alzheimer's Association, 2(3), Supplement S79.
Rosenfeld M. A., Yoshimura K., Trapnell B. C., Yoneyama K., Rosenthal E. R., Dalemans W., Fukayama M., Bargon J., Stier L. E., Stratford-Perricaudet L. et al. (1992). In vivo transfer of the human cystic fibrosis transmembrane conductance regulator gene to the airway epithelium. Cell, 68(1), 143 155.
Saaristo A., Partanen T. A., Arola J., et al. (2000). Vascular endothelial growth factor-C and its receptor VEGFR-3 in the nasal mucosa and in nasopharyngeal tumors. Am. J. Pathol., 157(1), 7-14.
Salven P., Lymboussaki A., Heikkila P., et al. (1998). Vascular endothelial growth factors VEGF-B and VEGF-C are expressed in human tumors. Am. J. Pathol., 153(1), 103-108.
Sambrook J., Fritsch E. F. & Maniatis T. (1989). Molecular cloning: a laboratory manual. Cold Spring Harbor Laboratory, Cold Spring Harbor, N.Y.
Scehnet J. S., Jiang W., Kumar S. R., et al. (2007). Inhibition of Dll4-mediated signaling induces proliferation of immature vessels and results in poor tissue perfusion. Blood, 109(11), 4753-4760.
Schweisguth, F. (2004). Regulation of notch signaling activity, Curr. Biol., 14(3), R129-138.
Segal D. J., Dreier B., Beerli R. R. & Barbas C. F. (1999). Toward controlling gene expression at will: selection and design of zinc finger domains recognizing each of the 5′-GNN-3′ DNA target sequences. Proc. Natl. Acad. Sci. USA., 96(6), 2758-2763.
Siemers E., Skinner M., Dean R. A., Gonzales C., Satterwhite J., Farlow M., Ness D. & May P. C. (2005). Safety, tolerability, and changes in amyloid beta concentrations after administration of a gamma-secretase inhibitor in volunteers. Clin Neuropharmacol., 28(3), 126-132.
Siironen P., Ristimaki A., Narko K., et al. (2006). VEGF-C and COX-2 expression in papillary thyroid cancer. Endocr. Relat. Cancer, 13(2), 465-473.
Skobe M., Hawighorst T., Jackson D. G., et al. (2001). Induction of tumor lymph-angiogenesis by VEGF-C promotes breast cancer metastasis. Nat. Med., 7(2), 192-198.
Smithies O., Gregg R. G., Boggs S. S., Koralewski M. A. & Kucherlapati R. S. (1985). Insertion of DNA sequences into the human chromosomal beta-globin locus by homologous recombination. Nature, 317(6034), 230-234.
Stacker S. A., Achen M. G., Jussila L., Baldwin M. E. & Alitalo K. (2002). Lymphangiogenesis and cancer metastasis. Nat. Rev. Cancer, 2(8), 573-583.
Stratford-Perricaudet L. D., Makeh I., Perricaudet M. & Briand P. (1992). Widespread long-term gene transfer to mouse skeletal muscles and heart. J. Clin. Invest., 90(2), 626 630.
Symons R. H. (1992). Small catalytic RNAs. Ann. Rev. Biochem., 61, 641-671.
Takeda S., Naito T., Hama K., Noma T. & Honjo T. (1985). Construction of chimaeric processed immunoglobulin genes containing mouse variable and human constant region sequences. Nature, 314(6010), 452-454.
Tammela T., Zarkada G., Wallgard E., et al. (2008). Blocking VEGFR-3 suppresses angiogenic sprouting and vascular network formation. Nature, 454(7204), 656-660.
Tammela T., Zarkada G., Nurmi H., et al. (2011). VEGFR-3 controls tip to stalk conversion at vessel fusion sites by reinforcing Notch signalling. Nat. Cell Biol., 13(10), 1202-1213.
Thomas K. R. & Capecchi M. R. (1987). Site-directed mutagenesis by gene targeting in mouse embryo-derived stem cells. Cell, 51(3), 503-512.
Thompson S., Clarke A. R., Pow A. M., Hooper M. L. & Melton D. W. (1989). Germ line transmission and expression of a corrected HPRT gene produced by gene targeting in embryonic stem cells. Cell, 56(2), 313-321.
Tijssen, P., Practice and Theory of immunoassays: Laboratory Techniques in Biochemistry and Molecular Biology, Elsevier Science Publishers, Amsterdam, The Netherlands (1985).
Tuschl T. (2002). Expanding small RNA interference. Nat. Biotechnol., 20(5), 446-448.
Usman N., Beigelman L. & McSwiggen J. A. (1993). Hammerhead ribozyme engineering. Current Opin. Struct. Biol., 6(4), 527-533.
Wu H., Yang W. P. & Barbas C. F. (1995). Building zinc fingers by selection: toward a therapeutic application. Proc. Natl. Acad. Sci. USA, 92(2), 344-348.
Yan M., Callahan C. A., Beyer J. C., et al. (2010). Chronic DLL4 blockade induces vascular neoplasms. Nature, 463(7282), E6-7.
Yu J. Y., DeRuiter S. L. & Turner, D. L. (2002). RNA interference by expression of short-interfering RNAs and hairpin RNAs in mammalian cells. Proc. Natl. Acad. Sci. USA., 99(9), 6047-6052.
Zeng Y., Wagner E. J. & Cullen B. R. (2002). Both natural and designed micro RNAs can inhibit the expression of cognate mRNAs when expressed in human cells. Mol. Cell, 9(6), 1327-1333.
Zheng W., Tammela T., Yamamoto M., et al. (2011). Notch restricts lymphatic vessel sprouting induced by vascular endothelial growth factor. Blood, 118(4), 1154-1162.

1. A method of screening for a mammalian subject with cancer to identify a subject for whom a Notch-targeted therapy will have efficacy, the method comprising:
measuring Vascular Endothelial Growth Factor-C (VEGF-C) expression in a biological sample from a mammalian subject with cancer, and
identifying or selecting a subject as one for whom a Notch-targeted therapy will have efficacy from the measurement of VEGF-C, wherein elevated VEGF-C expression in the sample identifies the subject as a subject for whom a Notch-targeted therapy will have efficacy.

2. The method of claim 1, wherein the cancer is a solid tumor.

3. The method of claim 2, wherein the biological sample comprises a tumor biopsy, and the VEGF-C is measured in the tumor.

4. The method according to claim 3, comprising comparing VEGF-C expression in the tumor with VEGF-C expression in healthy tissue of the same tissue type as the tumor, wherein elevated VEGF-C expression in the tumor compared to the healthy tissue identifies the subject as a subject for whom a Notch-targeted therapy will have efficacy.

5. The method of claim 2, wherein the sample includes tumor blood or lymphatic vessel tissue, and the VEGF-C is measured in vessel tissue.

6. The method according to claim 5, comprising comparing VEGF-C expression in the vessel tissue with VEGF-C expression in healthy vessel tissue of from the same tissue type as the tumor, wherein elevated VEGF-C expression in the vessel tissue from the tumor compared to the vessel tissue from healthy tissue identifies the subject as a subject for whom a Notch-targeted therapy will have efficacy.

7. The method of claim 2, wherein the sample includes fluid from the tumor, and the VEGF-C is measured in the fluid.

8. The method according to claim 7, comprising comparing VEGF-C expression in the tumor fluid with VEGF-C expression in fluid from healthy tissue of the same tissue type as the tumor, wherein elevated VEGF-C expression in the fluid from the tumor compared to the fluid from the healthy tissue identifies the subject as a subject for whom a Notch-targeted therapy will have efficacy.

9. The method of claim 1, wherein the biological sample comprises blood, and the VEGF-C is measured in the blood, or in plasma or serum from the blood.

10. The method according to claim 1, wherein a VEGF-C measurement that is at least 1.0 standard deviation, or at least 1.5 standard deviations, or at least 2.0 standard deviations, or at least 2.5 standard deviations, or at least 3 standard deviations greater than a median VEGF-C measurement in corresponding healthy tissue is scored as elevated VEGF-C expression.

11. The method according to claim 1, wherein a VEGF-C measurement that is statistically significantly greater than VEGF-C measurements in corresponding healthy tissue, with a p-value less than 0.1, or less than 0.05, or less than 0.01, or less than 0.005, or less than 0.001 is scored as elevated VEGF-C expression.

12. The method according to claim 1, wherein the identifying or selecting a subject comprises comparing the measurement of VEGF-C to a reference measurement of VEGF-C, and scoring the VEGF-C measurement from the sample as elevated based on statistical analysis or a ratio relative to the reference measurement.

13. The method according to claim 12, wherein the reference measurement comprises at least one of the following:
(a) a measurement of VEGF-C from healthy tissue of the subject of the same tissue type as the sample;
(b) a database containing multiple VEGF-C measurements from healthy or cancerous tissues from other subjects; and
(c) a reference value calculated from multiple VEGF-C measurements from healthy or cancerous tissues from other subjects, optionally further including statistical distribution information for the multiple measurements, such as standard deviation.

14. The method according to claim 1, further comprising a step, prior to said measuring step, of obtaining the biological sample from a mammalian subject.

15. The method according to claim 1, wherein the tumor is a tumor of a tissue or organ selected from the group consisting of colon, rectum, intestine, breast, ovary, lung, stomach, brain, pancreas, ovary, prostate, kidney, liver, and head and neck

16. The method according to claim 1, wherein the tumor is selected from the group consisting of colorectal cancer, breast cancer, lung cancer, gastric cancer, glioblastoma, and pancreatic cancer.

17. The method according to claim 1, wherein the measuring comprises measuring VEGF-C protein in the biological sample.

18. The method according to claim 17, wherein the measuring comprises an immunohistochemical assay.

19. The method of claim 17, wherein the measuring comprises contacting the biological sample with a VEGF-C antibody or antigen-binding fragment thereof, and measuring the amount of VEGF-C antibody complex formed.

20. The method according to claim 19, wherein the antibody comprises a label.

21. The method of claim 17, wherein the measuring comprises contacting the biological sample with a polypeptide comprising an extracellular domain fragment of VEGFR-3 that binds VEGF-C, and measuring the amount of VEGF-C/VEGFR-3 complex formed.

22. The method of any claim 1, wherein the measuring comprises measuring VEGF-C mRNA in the biological sample.

23. The method of claim 22, wherein the measuring comprises in situ hybridization to measure the quantity and/or distribution of VEGF-C mRNA in the biological sample.

24. The method of claim 22, wherein the measuring comprises steps of isolating mRNA from the biological sample and measuring VEGF-C mRNA in the isolated mRNA.

25. The method according to claim 23, wherein the measuring comprises polymerase chain reaction (PCR) to quantify VEGF-C mRNA in the biological sample relative to VEGF-C mRNA in a corresponding healthy biological sample.

26. The method according to claim 25, wherein the PCR includes a procedure selected from reverse transcriptase PCR, real time PCR, and quantitative PCR.

27. A method according to claim 1, further comprising measuring expression of at least one additional marker selected from the group consisting of HES1, HES4, HES5, HESL, HEY-2, DTX1, MYC, NRARP, PTCRA, SHQ1, and HeyL (hairy/enhancer-of-split related with YRPW motif-like) in the biological sample, and
identifying the subject as a subject for whom a Notch-targeted therapy will be effective based on the measurements of VEGF-C and the at least one additional marker, wherein elevated VEGF-C expression and elevated expression of the at least one additional marker indicates the presence of a cancer for which Notch-targeted therapy will be effective.

28. The method according to claim 1, wherein the mammalian subject is a human.

29. The method according to claim 1, further comprising a step of prescribing for or administering to subject identified as having the elevated VEGF-C expression in the biological sample a composition comprising a molecule that suppresses expression or downstream signaling activity of Notch (“Notch inhibitor”).

30. A method of treatment comprising:
obtaining a tumor or tumor biopsy from a human subject;
determining that the tumor or tumor biopsy has elevated expression of VEGF-C; and
prescribing for or administering to the subject a composition comprising a molecule that suppresses expression or signaling activity of Notch (“Notch inhibitor”).

31. The method of claim 30, wherein the determining step comprises ordering a laboratory test that measures VEGF-C in the tumor or tumor biopsy and learning the measurement from a report from the laboratory.

32. The method of claim 30, wherein the determining step comprises measuring VEGF-C mRNA or VEGF-C protein in the tumor or tumor biopsy.

33-34. (canceled)

35. The method according to claim 30, wherein the composition further comprises a pharmaceutically acceptable diluent, adjuvant, or carrier medium.

36. The method according to claim 30, wherein the Notch inhibitor is selected from the group consisting of:
(a) an antibody that binds a Notch protein and inhibits ligand-mediated stimulation of Notch signaling;
(b) a soluble polypeptide that comprises an extracellular domain fragment of a Notch polypeptide that binds a Notch ligand and inhibits the ligand from stimulation of Notch signaling;
(c) an antisense or interfering nucleic acid (e.g., antisense oligonucleotide; micro-RNA, short interfering RNA) that inhibits Notch expression;
(d) an antibody that binds a Notch ligand protein and inhibits the ligand from stimulation of Notch signaling;
(e) a soluble notch ligand polypeptide that comprises an extracellular domain fragment of a Notch ligand that binds Notch and inhibits stimulation of Notch by ligand expressed by a cell;
(f) an antisense or interfering nucleic acid (e.g., antisense oligonucleotide; micro-RNA, short interfering RNA) that inhibits expression of a Notch ligand;
(g) a small molecule that inhibits Notch expression or signaling;
(h) a molecule that inhibits proteolytic cleavage-activation of Notch; and
(i) a molecule that inhibits Notch NICD peptide from binding core binding factor-1 (CBF-1) or activating transcription of one or more genes selected from HES, Myc, and p21.

37. The method according to claim 36, wherein the Notich inhibitor is selected from the group consisting of:
(a) an antibody that binds a Notch protein and inhibits delta-like ligand 4 (Dll4) stimulation of Notch signaling;
(b) a soluble polypeptide that comprises an extracellular domain fragment of a Notch polypeptide that binds Dll4 and inhibits Dll4 stimulation of Notch signaling;
(c) an antisense or interfering nucleic acid (e.g., antisense oligonucleotide; micro-RNA, short interfering RNA) that inhibits Notch expression;
(d) an antibody that binds a Dll4 protein and inhibits Dll4 stimulation of Notch signaling;
(e) a soluble Dll4 polypeptide that comprises an extracellular domain fragment of Dll4 that binds Notch and inhibits stimulation of Notch by cellular Dll4; and
(f) an antisense or interfering nucleic acid (e.g., antisense oligonucleotide; micro-RNA, short interfering RNA) that inhibits Dll4 expression.

38. The method according to claim 36, wherein the Notch inhibitor is an inhibitor of Dll4 stimulation of Notch4 signaling.

39. The method according to claim 35, wherein the Notch inhibitor comprises the extracellular domain fragment of the Notch polypeptide or Notch ligand fused to an immunoglobulin constant domain fragment (Fc).

40. The method according to claim 30, wherein the Notch inhibitor is selected from the group consisting of: inhibitors of the TNFα converting enzymes (TACE inhibitors), such as ADAM10 and ADAM17; and inhibitors of gamma-secretase.

41. The method according to claim 30, wherein the Notch inhibitor is a gamma secretase inhibitor.

42. The method according to claim 30, wherein the Notch inhibitor is selected from the inhibitors set for in Table 1A.

43. The method according to claim 30, further comprising administering to the subject a composition comprising a standard-of-care cancer therapeutic for the subject's cancer.

44. (canceled)

45. The method according to claim 1, wherein the measuring or determining of VEGF-C occurs after cancer diagnosis and prior to initiation of a chemotherapy.

46. The method according to claim 1, wherein the measuring or determining of VEGF-C occurs after a cancer has become resistant to a chemotherapy.

47. A system for identifying a human subject with cancer as a subject for whom a Notch-targeted therapy will have efficacy, the system comprising:
(a) at least one processor;
(b) at least one computer-readable medium;
(c) a database operatively coupled to a computer-readable medium of the system and containing population information correlating the measurement of VEGF-C expression and efficacy of Notch-targeted therapy data in a population of humans with cancer;
(d) a measurement tool that receives an input about the human subject and generates information from the input about the measurement of VEGF-C expression from the human subject; and
(e) an analysis tool or routine that:
(i) is operatively coupled to the database and the measurement tool,
(ii) is stored on a computer-readable medium of the system,
(iii) is adapted to be executed on a processor of the system, to compare the information about the human subject with the population information in the database and generate a conclusion with respect to a likelihood of efficacy of Notch-targeted therapy in the human subject.


SEQUENCE LISTING (24 sequences)

SEQ ID NO: 1 - DNA, 2076 bp, Homo sapiens
cggggaaggggagggaggagggggacgagggctctggcgggtttggaggggctgaacatcgcggggtgttctggtgtcccccgccccgcctctccaaaaagctacaccgacgcggaccgcggcggcgtcctccctcgccctcgcttcacctcgcgggctccgaatgcggggagctcggatgtccggtttcctgtgaggcttttacctgacacccgccgcctttccccggcactggctgggagggcgccctgcaaagttgggaacgcggagccccggacccgctcccgccgcctccggctcgcccagggggggtcgccgggaggagcccgggggagagggaccaggaggggcccgcggcctcgcaggggcgcccgcgcccccacccctgcccccgccagcggaccggtcccccacccccggtccttccaccatgcacttgctgggcttcttctctgtggcgtgttctctgctcgccgctgcgctgctcccgggtcctcgcgaggcgcccgccgccgccgccgccttcgagtccggactcgacctctcggacgcggagcccgacgcgggcgaggccacggcttatgcaagcaaagatctggaggagcagttacggtctgtgtccagtgtagatgaactcatgactgtactctacccagaatattggaaaatgtacaagtgtcagctaaggaaaggaggctggcaacataacagagaacaggccaacctcaactcaaggacagaagagactataaaatttgctgcagcacattataatacagagatcttgaaaagtattgataatgagtggagaaagactcaatgcatgccacgggaggtgtgtatagatgtggggaaggagtttggagtcgcgacaaacaccttctttaaacctccatgtgtgtccgtctacagatgtgggggttgctgcaatagtgaggggctgcagtgcatgaacaccagcacgagctacctcagcaagacgttatttgaaattacagtgcctctctctcaaggccccaaaccagtaacaatcagttttgccaatcacacttcctgccgatgcatgtctaaactggatgtttacagacaagttcattccattattagacgttccctgccagcaacactaccacagtgtcaggcagcgaacaagacctgccccaccaattacatgtggaataatcacatctgcagatgcctggctcaggaagattttatgttttcctcggatgctggagatgactcaacagatggattccatgacatctgtggaccaaacaaggagctggatgaagagacctgtcagtgtgtctgcagagcggggcttcggcctgccagctgtggaccccacaaagaactagacagaaactcatgccagtgtgtctgtaaaaacaaactcttccccagccaatgtggggccaaccgagaatttgatgaaaacacatgccagtgtgtatgtaaaagaacctgccccagaaatcaacccctaaatcctggaaaatgtgcctgtgaatgtacagaaagtccacagaaatgcttgttaaaaggaaagaagttccaccaccaaacatgcagctgttacagacggccatgtacgaaccgccagaaggcttgtgagccaggattttcatatagtgaagaagtgtgtcgttgtgtcccttcatattggaaaagaccacaaatgagctaagattgtactgttttccagttcatcgattttctattatggaaaactgtgttgccacagtagaactgtctgtgaacagagagacccttgtgggtccatgctaacaaagacaaaagtctgtctttcctgaaccatgtggataactttacagaaatggactggagctcatctgcaaaaggcctcttgtaaagactggttttctgccaatgaccaaacagccaagattttcctcttgtgatttctttaaaagaatgactatataatttatttccactaaaaatattgtttctgcattcatttttatagcaacaacaattggtaaaactcactgtgatcaatatttttatatcatgcaaaatatgtttaaaataaaatgaaaattgtattat

SEQ ID NO: 2 - protein, 419 aa, Homo sapiens
MHLLGFFSVACSLLAAALLPGPREAPAAAAAFESGLDLSDAEPDAGEATAYASKDLEEQLRSVSSVDELMTVLYPEYWKMYKCQLRKGGWQHNREQANLNSRTEETIKFAAAHYNTEILKSIDNEWRKTQCMPREVCIDVGKEFGVATNTFFKPPCVSVYRCGGCCNSEGLQCMNTSTSYLSKTLFEITVPLSQGPKPVTISFANHTSCRCMSKLDVYRQVHSIIRRSLPATLPQCQAANKTCPTNYMWNNHICRCLAQEDFMFSSDAGDDSTDGFHDICGPNKELDEETCQCVCRAGLRPASCGPHKELDRNSCQCVCKNKLFPSQCGANREFDENTCQCVCKRTCPRNQPLNPGKCACECTESPQKCLLKGKKFHHQTCSCYRRPCTNRQKACEPGFSYSEEVCRCVPSYWKRPQMS

SEQ ID NO: 3 - DNA, 7693 bp, Homo sapiens
atgccgccgctcctggcgcccctgctctgcctggcgctgctgcccgcgctcgccgcacgaggcccgcgatgctcccagcccggtgagacctgcctgaatggcgggaagtgtgaagcggccaatggcacggaggcctgcgtctgtggcggggccttcgtgggcccgcgatgccaggaccccaacccgtgcctcagcaccccctgcaagaacgccgggacatgccacgtggtggaccgcagaggcgtggcagactatgcctgcagctgtgccctgggcttctctgggcccctctgcctgacacccctggacaacgcctgcctcaccaacccctgccgcaacgggggcacctgcgacctgctcacgctgacggagtacaagtgccgctgcccgcccggctggtcagggaaatcgtgccagcaggctgacccgtgcgcctccaacccctgcgccaacggtggccagtgcctgcccttcgaggcctcctacatctgccactgcccacccagcttccatggccccacctgccggcaggatgtcaacgagtgtggccagaagcccaggctttgccgccacggaggcacctgccacaacgaggtcggctcctaccgctgcgtctgccgcgccacccacactggccccaactgcgagcggccctacgtgccctgcagcccctcgccctgccagaacgggggcacctgccgccccacgggcgacgtcacccacgagtgtgcctgcctgccaggcttcaccggccagaactgtgaggaaaatatcgacgattgtccaggaaacaactgcaagaacgggggtgcctgtgtggacggcgtgaacacctacaactgcccgtgcccgccagagtggacaggtcagtactgtaccgaggatgtggacgagtgccagctgatgccaaatgcctgccagaacggcgggacctgccacaacacccacggtggctacaactgcgtgtgtgtcaacggctggactggtgaggactgcagcgagaacattgatgactgtgccagcgccgcctgcttccacggcgccacctgccatgaccgtgtggcctccttttactgcgagtgtccccatggccgcacaggtctgctgtgccacctcaacgacgcatgcatcagcaacccctgtaacgagggctccaactgcgacaccaaccctgtcaatggcaaggccatctgcacctgcccctcggggtacacgggcccggcctgcagccaggacgtggatgagtgctcgctgggtgccaacccctgcgagcatgcgggcaagtgcatcaacacgctgggctccttcgagtgccagtgtctgcagggctacacgggcccccgatgcgagatcgacgtcaacgagtgcgtctcgaacccgtgccagaacgacgccacctgcctggaccagattggggagttccagtgcatgtgcatgcccggctacgagggtgtgcactgcgaggtcaacacagacgagtgtgccagcagcccctgcctgcacaatggccgctgcctggacaagatcaatgagttccagtgcgagtgccccacgggcttcactgggcatctgtgccagtacgatgtggacgagtgtgccagcaccccctgcaagaatggtgccaagtgcctggacggacccaacacttacacctgtgtgtgcacggaagggtacacggggacgcactgcgaggtggacatcgatgagtgcgaccccgacccctgccactacggctcctgcaaggacggcgtcgccaccttcacctgcctctgccgcccaggctacacgggccaccactgcgagaccaacatcaacgagtgctccagccagccctgccgcctacggggcacctgccaggacccggacaacgcctacctctgcttctgcctgaaggggaccacaggacccaactgcgagatcaacctggatgactgtgccagcagcccctgcgactcgggcacctgtctggacaagatcgatggctacgagtgtgcctgtgagccgggctacacagggagcatgtgtaacagcaacatcgatgagtgtgcgggcaacccctgccacaacgggggcacctgcgaggacggcatcaatggcttcacctgccgctgccccgagggctaccacgaccccacctgcctgtctgaggtcaatgagtgcaacagcaacccctgcgtccacggggcctgccgggacagcctcaacgggtacaagtgcgactgtgaccctgggtggagtgggaccaactgtgacatcaacaacaacgagtgtgaatccaacccttgtgtcaacggcggcacctgcaaagacatgaccagtggcatcgtgtgcacctgccgggagggcttcagcggtcccaactgccagaccaacatcaacgagtgtgcgtccaacccatgtctgaacaagggcacgtgtattgacgacgttgccgggtacaagtgcaactgcctgctgccctacacaggtgccacgtgtgaggtggtgctggccccgtgtgcccccagcccctgcagaaacggcggggagtgcaggcaatccgaggactatgagagcttctcctgtgtctgccccacggctggggccaaagggcagacctgtgaggtcgacatcaacgagtgcgttctgagcccgtgccggcacggcgcatcctgccagaacacccacggcgsstaccgctgccactgccaggccggctacagtgggcgcaactgcgagaccgacatcgacgactgccggcccaacccgtgtcacaacgggggctcctgcacagacggcatcaacacggccttctgcgactgcctgcccggcttccggggcactttctgtgaggaggacatcaacgagtgtgccagtgacccctgccgcaacggggccaactgcacggactgcgtggacagctacacgtgcacctgccccgcaggcttcagcgggatccactgtgagaacaacacgcctgactgcacagagagctcctgcttcaacggtggcacctgcgtggacggcatcaactcgttcacctgcctgtgtccacccggcttcacgggcagctactgccagcacgtagtcaatgagtgcgactcacgaccctgcctgctaggcggcacctgtcaggacggtcgcggtctccacaggtgcacctgcccccagggctacactggccccaactgccagaaccttgtgcactggtgtgactcctcgccctgcaagaacggcggcaaatgctggcagacccacacccagtaccgctgcgagtgccccagcggctggaccggcctttactgcgacgtgcccagcgtgtcctgtgaggtggctgcgcagcgacaaggtgttgacgttgcccgcctgtgccagcatggagggctctgtgtggacgcgggcaacacgcaccactgccgctgccaggcgggctacacaggcagctactgtgaggacctggtggacgagtgctcacccagcccctgccagaacggggccacctgcacggactacctgggcggctactcctgcaagtgcgtggccggctaccacggggtgaactgctctgaggagatcgacgagtgcctctcccacccctgccagaacgggggcacctgcctcgacctccccaacacctacaagtgctcctgcccacggggcactcagggtgtgcactgtgagatcaacgtggacgactgcaatccccccgttgaccccgtgtcccggagccccaagtgctttaacaacggcacctgcgtggaccaggtgggcggctacagctgcacctgcccgccgggcttcgtgggtgagcgctgtgagggggatgtcaacgagtgcctgtccaatccctgcgacgcccgtggcacccagaactgcgtgcagcgcgtcaatgacttccactgcgagtgccgtgctggtcacaccgggcgccgctgcgagtccgtcatcaatggctgcaaaggcaagccctgcaagaatgggggcacctgcgccgtggcctccaacaccgcccgcgggttcatctgcaagtgccctgcgggcttcgagggcgccacgtgtgagaatgacgctcgtacctgcggcagcctgcgctgcctcaacggcggcacatgcatctccggcccgcgcagccccacctgcctgtgcctgggccccttcacgggccccgaatgccagttcccggccagcagcccctgcctgggcggcaacccctgctacaaccaggggacctgtgagcccacatccgagagccccttctaccgttgcctgtgccccgccaaattcaacgggctcttgtgccacatcctggactacagcttcgggggtggggccgggcgcgacatccccccgccgctgatcgaggaggcgtgcgagctgcccgagtgccaggaggacgcgggcaacaaggtctgcagcctgcagtgcaacaaccacgcgtgcggctgggacggcggtgactgctccctcaacttcaatgacccctggaagaactgcacgcagtctctgcagtgctggaagtacttcagtgacggccactgtgacagccagtgcaactcagccggctgcctcttcgacggctttgactgccagcgtgcggaaggccagtgcaaccccctgtacgaccagtactgcaaggaccacttcagcgacgggcactgcgaccagggctgcaacagcgcggagtgcgagtgggacgggctggactgtgcggagcatgtacccgagaggctggcggccggcacgctggtggtggtggtgctgatgccgccggagcagctgcgcaacagctccttccacttcctgcgggagctcagccgcgtgctgcacaccaacgtggtcttcaagcgtgacgcacacggccagcagatgatcttcccctactacggccgcgaggaggagctgcgcaagcaccccatcaagcgtgccgccgagggctgggccgcacctgacgccctgctgggccaggtgaaggcctcgctgctccctggtggcagcgagggtgggcggcggcggagggagctggaccccatggacgtccgcggctccatcgtctacctggagattgacaaccggcagtgtgtgcaggcctcctcgcagtgcttccagagtgccaccgacgtggccgcattcctgggagcgctcgcctcgctgggcagcctcaacatcccctacaagatcgaggccgtgcagagtgagaccgtggagccgcccccgccggcgcagctgcacttcatgtacgtggcggcggccgcctttgtgcttctgttcttcgtgggctgcggggtgctgctgtcccgcaagcgccggcggcagcatggccagctctggttccctgagggcttcaaagtgtctgaggccagcaagaagaagcggcgggagcccctcggcgaggactccgtgggcctcaagcccctgaagaacgcttcagacggtgccctcatggacgacaaccagaatgagtggggggacgaggacctggagaccaagaagttccggttcgaggagcccgtggttctgcctgacctggacgaccagacagaccaccggcagtggactcagcagcacctggatgccgctgacctgcgcatgtctgccatggcccccacaccgccccagggtgaggttgacgccgactgcatggacgtcaatgtccgcgggcctgatggcttcaccccgctcatgatcgcctcctgcagcgggggcggcctggagacgggcaacagcgaggaagaggaggacgcgccggccgtcatctccgacttcatctaccagggcgccagcctgcacaaccagacagaccgcacgggcgagaccgccttgcacctggccgcccgctactcacgctctgatgccgccaagcgcctgctggaggccagcgcagatgccaacatccaggacaacatgggccgcaccccgctgcatgcggctgtgtctgccgacgcacaaggtgtcttccagatcctgatccggaaccgagccacagacctggatgcccgcatgcatgatggcacgacgccactgatcctggctgcccgcctggccgtggagggcatgctggaggacctcatcaactcacacgccgacgtcaacgccgtagatgacctgggcaagtccgccctgcactgggccgccgccgtgaacaatgtggatgccgcagttgtgctcctgaagaacggggctaacaaagatatgcagaacaacagggaggagacacccctgtttctggccgcccgggagggcagctacgagaccgccaaggtgctgctggaccactttgccaaccgggacatcacggatcatatggaccgcctgccgcgcgacatcgcacaggagcgcatgcatcacgacatcgtgaggctgctggacgagtacaacctggtgcgcagcccgcagctgcacggagccccgctggggggcacgcccaccctgtcgcccccgctctgctcgcccaacggctacctgggcagcctcaagcccggcgtgcagggcaagaaggtccgcaagcccagcagcaaaggcctggcctgtggaagcaaggaggccaaggacctcaaggcacggaggaagaagtcccaggatggcaagggctgcctgctggacagctccggcatgctctcgcccgtggactccctggagtcaccccatggctacctgtcagacgtggcctcgccgccactgctgccctccccgttccagcagtctccgtccgtgcccctcaaccacctgcctgggatgcccgacacccacctgggcatcgggcacctgaacgtggcggccaagcccgagatggcggcgctgggtgggggcggccggctggcctttgagactggcccacctcgtctctcccacctgcctgtggcctctggcaccagcaccgtcctgggctccagcagcggaggggccctgaatttcactgtgggcgggtccaccagtttgaatggtcaatgcgagtggctgtcccggctgcagagcggcatggtgccgaaccaatacaaccctctgcgggggagtgtggcaccaggccccctgagcacacaggccccctccctgcagcatggcatggtaggcccgctgcacagtagccttgctgccagcgccctgtcccagatgatgagctaccagggcctgcccagcacccggctggccacccagcctcacctggtgcagacccagcaggtgcagccacaaaacttacagatgcagcagcagaacctgcagccagcaaacatccagcagcagcaaagcctgcagccgccaccaccaccaccacagccgcaccttggcgtgagctcagcagccagcggccacctgggccggagcttcctgagtggagagccgagccaggcagacgtgcagccactgggccccagcagcctggcggtgcacactattctgccccaggagagccccgccctgcccacgtcgctgccatcctcgctggtcccacccgtgaccgcagcccagttcctgacgcccccctcgcagcacagctactcctcgcctgtggacaacacccccagccaccagctacaggtgcctgagcaccccttcctgaccccttcgccggagtcgcccgaccaatggtcgtcctcgtcgccgcactctaatgtgtctgactggtctgagggcgtgtcgtcgcccccgacctccatgcagtcccagatcgcgcgcatcccggaggcgttcaagtaatagctcgaggtgccagcagctc

SEQ ID NO: 4 - protein, 2555 aa, Homo sapiens
MPPLLAPLLCLALLPALAARGPRCSQPGETCLNGGKCEAANGTEACVCGGAFVGPRCQDPNPCLSTPCKNAGTCHVVDRRGVADYACSCALGFSGPLCLTPLDNACLTNPCRNGGTCDLLTLTEYKCRCPPGWSGKSCQQADPCASNPCANGGQCLPFEASYICHCPPSFHGPTCRQDVNECGQKPGLCRHGGTCHNEVGSYRCVCRATHTGPNCERPYVPCSPSPCQNGGTCRPTGDVTHECACLPGFTGQNCEENIDDCPGNNCKNGGACVDGVNTYNCRCPPEWTGQYCTEDVDECQLMPNACQNGGTCHNTHGGYNCVCVNGWTGEDCSENIDDCASAACFHGATCHDRVASFYCECPHGRTGLLCHLNDACISNPCNEGSNCDTNPVNGKAICTCPSGYTGPACSQDVDECSLGANPCEHAGKCINTLGSFECQCLQGYTGPRCEIDVNECVSNPCQNDATCLDQIGEFQCICMPGYEGVHCEVNTDECASSPCLHNGRCLDKINEFQCECPTGFTGHLCQYDVDECASTPCKNGAKCLDGPNTYTCVCTEGYTGTHCEVDIDECDPDPCHYGSCKDGVATFTCLCRPGYTGHHCETNINECSSQPCRHGGTCQDRDNAYLCFCLKGTTGPNCEINLDDCASSPCDSGTCLDKIDGYECACEPGYTGSMCNINIDECAGNPCHNGGTCEDGINGFTCRCPEGYHDPTCLSEVNECNSNPCVHGACRDSLNGYKCDCDPGWSGTNCDINNNECESNPCVNGGTCKDMTSGYVCTCREGFSGPNCQTNINECASNPCLNQGTCIDDVAGYKCNCLLPYTGATCEVVLAPCAPSPCRNGGECRQSEDYESFSCVCPTGWQGQTCEVDINECVLSPCRHGASCQNTHGGYRCHCQAGYSGRNCETDIDDCRPNPCHNGGSCTDGINTAFCDCLPGFRGTFCEEDINECASDPCRNGANCTDCVDSYTCTCPAGFSGIHCENNTPDCTESSCFNGGTCVDGINSFTCLCPPGFTGSYCQHDVNECDSQPCLHGGTCQDGCGSYRCTCPQGYTGPNCQNLVHWCDSSPCKNGGKCWQTHTQYRCECPSGWTGLYCDVPSVSCEVAAQRQGVDVARLCQHGGLCVDAGNTHHCRCQAGYTGSYCEDLVDECSPSPCQNGATCTDYLGGYSCKCVAGYHGVNCSEEIDECLSHPCQNGGTCLDLPNTYKCSCPRGTQGVHCEINVDDCNPPVDPVSRSPKCFNNGTCVDQVGGYSCTCPPGFVGERCEGDVNECLSNPCDARGTQNCVQRVNDFHCECRAGHTGRRCESVINGCKGKPCKNGGTCAVASNTARGFICKCPAGFEGATCENDARTCGSLRCLNGGTCISGPRSPTCLCLGPFTGPECQFPASSPCLGGNPCYNQGTCEPTSESPFYRCLCPAKFNGLLCHILDYSFGGGAGRDIPPPLIEEACELPECQEDAGNKVCSLQCNNHACGWDGGDCSLNFNDPWKNCTQSLQCWKYFSDGHCDSQCNSAGCLFDGFDCQRAEGQCNPLYDQYCKDHFSDGHCDQGCNSAECEWDGLDCAEHVPERLAAGTLVVVVLMPPEQLRNSSFHFLRELSRVLHTNVVFKRDAHGQQMIFPYYGREEELRKHPIKRAAEGWAAPDALLGQVKASLLPGGSEGGRRRRELDPMDVRGSIVYLEIDNRQCVQASSQCFQSATDVAAFLGALASLGSLNIPYKIEAVQSETVEPPPPAQLHFMYVAAAAFVLLFFVGCGVLLSRKRRRQHGQLWFPEGFKVSEASKKKRREPLGEDSVGLKPLKNASDGALMDDNQNEWGDEDLETKKFRFEEPVVLPDLDDQTDHRQWTQQHLDAADLRMSAMAPTPPQGEVDADCMDVNVRGPDGFTPLMIASCSGGGLETGNSEEEEDAPAVISDFIYQGASLHNQTDRTGETALHLAARYSRSDAAKRLLEASADANIQDNMGRTPLHAAVSADAQGVFQILIRNRATDLDARMHDGTTPLILAARLAVEGMLEDLINSHADVNAVDDLGKSALHWAAAVNNVDAAVVLLKNGANKDMQNNREETPLFLAAREGSYETAKVLLDHFANRDITDHMDRLPRDIAQERMHHDIVRLLDEYNLVRSPQLHGAPLGGTPTLSPPLCSPNGYLGSLKPGVQGKKVRKPSSKGLACGSKEAKDLKARRKKSQDGKGCLLDSSGMLSPVDSLESPHGYLSDVASPPLLPSPFQQSPSVPLNHLPGMPDTHLGIGHLNVAAKPEMAALGGGGRLAFETGPPRLSHLPVASGTSTVLGSSSGGALNFTVGGSTSLNGQCEWLSRLQSGMVPNQYNPLRGSVAPGPLSTQAPSLQHGMVGPLHSSLAASALSQMMSYQGLPSTRLATQPHLVQTQQVQPQNLQMQQQNLQPANIQQQQSLQPPPPPPQPHLGVSSAASGHLGRSFLSGEPSQADVQPLGPSSLAVHTILPQESPALPTSLPSSLVPPVTAAQFLTPPSQHSYSSPVDNTPSHQLQVPEHPFLTPSPESPDQWSSSSPHSNVSDWSEGVSSPPTSMQSQIARIPEAFK

SEQ ID NO: 5 - DNA, 9722 bp, Homo sapiens
tcatctggaattatgcccgccctgcgccccgctctgctgtgggcgctgctggcgctctggctgtgctgcgcggcccccgcgcatgcattgcagtgtcgagatggctatgaaccctgtgtaaatgaaggaatgtgtgttacctaccacaatggcacaggatactgcaaatgtccagaaggcttcttgggggaatattgtcaacatcgagacccctgtgagaagaaccgctgccagaatggtgggacttgtgtggcccaggccatgctggggaaagccacgtgccgatgtgcctcagggtttacaggagaggactgccagtactcaacatctcatccatgctttgtgtctcgaccctgcctgaatggcggcacatgccatatgctcagccgggatacctatgagtgcacctgtcaagtcgggtttacaggtaaggagtgccaatggacggatgcctgcctgtctcatccctgtgcaaatggaagtacctgtaccactgtggccaaccagttctcctgcaaatgcctcacaggcttcacagggcagaaatgtgagactgatgtcaatgagtgtgacattccaggacactgccagcatggtggcacctgcctcaacctgcctggttcctaccagtgccagtgccctcagggcttcacaggccagtactgtgacagcctgtatgtgccctgtgcaccctcaccttgtgtcaatggaggcacctgtcggcagactggtgacttcacttttgagtgcaactgccttccaggttttgaagggagcacctgtgagaggaatattgatgactgccctaaccacaggtgtcagaatggaggggtttgtgtggatggggtcaacacttacaactgccgctgtcccccacaatggacaggacagttctgcacagaggatgtggatgaatgcctgctgcagcccaatgcctgtcaaaatgggggcacctgtgccaaccgcaatggaggctatggctgtgtatgtgtcaacggctggagtggagatgactgcagtgagaacattgatgattgtgccttcgcctcctgtactccaggctccacctgcatcgaccgtgtggcctccttctcttgcatgtgcccagaggggaaggcaggtctcctgtgtcatctggatgatgcatgcatcagcaatccttgccacaagggggcactgtgtgacaccaaccccctaaatgggcaatatatttgcacctgcccacaaggctacaaaggggctgactgcacagaagatgtggatgaatgtgccatggccaatagcaatccttgtgagcatgcaggaaaatgtgtgaacacggatggcgccttccactgtgagtgtctgaagggttatgcaggacctcgttgtgagatggacatcaatgagtgccattcagacccctgccagaatgatgctacctgtctggataagattggaggcttcacatgtctgtgcatgccaggtttcaaaggtgtgcattgtgaattagaaataaatgaatgtcagagcaacccttgtgtgaacaatgggcagtgtgtggataaagtcaatcgtttccagtgcctgtgtcctcctggtttcactgggccagtttgccagattgatattgatgactgttccagtactccgtgtctgaatggggcaaagtgtatcgatcacccgaatggctatgaatgccagtgtgccacaggtttcactggtgtgttgtgtgaggagaacattgacaactgtgaccccgatccttgccaccatggtcagtgtcaggatggtattgattcctacacctgcatctgcaatcccgggtacatgggcgccatctgcagtgaccagattgatgaatgttacagcagcccttgcctgaacgatggtcgctgcattgacctggtcaatggctaccagtgcaactgccagccaggcacgtcaggggttaattgtgaaattaattttgatgactgtgcaagtaacccttgtatccatggaatctgtatggatggcattaatcgctacagttgtgtctgctcaccaggattcacagggcagagatgtaacattgacattgatgagtgtgcctccaatccctgtcgcaagggtgcaacatgtatcaacggtgtgaatggtttccgctgtatatgccccgagggaccccatcaccccagctgctactcacaggtgaacgaatgcctgagcaatccctgcatccatggaaactgtactggaggtctcagtggatataagtgtctctgtgatgcaggctgggttggcatcaactgtgaagtggacaaaaatgaatgcctttcgaatccatgccagaatggaggaacttgtgacaatctggtgaatggatacaggtgtacttgcaagaagggctttaaaggctataactgccaggtgaatattgatgaatgtgcctcaaatccatgcctgaaccaaggaacctgctttgatgacataagtggctacacttgccactgtgtgctgccatacacaggcaagaattgtcagacagtattggctccctgttccccaaacccttgtgagaatgctgctgtttgcaaagagtcaccaaattttgagagttatacttgcttgtgtgctcctggctggcaaggtcagcggtgtaccattgacattgacgagtgtatctccaagccctgcatgaaccatggtctctgccataacacccagggcagctacatgtgtgaatgtccaccaggcttcagtggtatggactgtgaggaggacattgatgactgccttgccaatccttgccagaatggaggttcctgtatggatggagtgaatactttctcctgcctctgccttccgggtttcactggggataagtgccagacagacatgaatgagtgtctgagtgaaccctgtaagaatggagggacctgctctgactacgtcaacagttacacttgcaagtgccaggcaggatttgatggagtccattgtgagaacaacatcaatgagtgcactgagagctcctgtttcaatggtggcacatgtgttgatgggattaactccttctcttgcttgtgccctgtgggtttcactggatccttctgcctccatgagatcaatgaatgcagctctcatccatgcctgaatgagggaacgtgtgttgatggcctgggtacctaccgctgcagctgccccctgggctacactgggaaaaactgtcagaccctggtgaatctctgcagtcggtctccatgtaaaaacaaaggtacttgtgttcagaaaaaagcagagtcccagtgcctatgtccatctggatgggctggtgcctattgtgacgtgcccaatgtctcttgtgacatagcagcctccaggagaggtgtgcttgttgaacacttgtgccagcactcaggtgtctgcatcaatgctggcaacacgcattactgtcagtgccccctgggctatactgggagctactgtgaggagcaactcgatgagtgtgcgtccaacccctgccagcacggggcaacatgcagtgacttcattggtggatacagatgcgagtgtgtcccaggctatcagggtgtcaactgtgagtatgaagtggatgagtgccagaatcagccctgccagaatggaggcacctgtattgaccttgtgaaccatttcaagtgctcttgcccaccaggcactcggggcctactctgtgaagagaacattgatgactgtgcccggggtccccattgccttaatggtggtcagtgcatggataggattggaggctacagttgtcgctgcttgcctggctttgctggggagcgttgtgagggagacatcaacgagtgcctctccaacccctgcagctctgagggcagcctggactgtatacagctcaccaatgactacctgtgtgtttgccgtagtgcctttactggccggcactgtgaaaccttcgtcgatgtgtgtccccagatgccctgcctgaatggagggacttgtgctgtggccagtaacatgcctgatggtttcatttgccgttgtcccccgggattttccggggcaaggtgccagagcagctgtggacaagtgaaatgtaggaagggggagcagtgtgtgcacaccgcctctggaccccgctgcttctgccccagtccccgggactgcgagtcaggctgtgccagtagcccctgccagcacgggggcagctgccaccctcagcgccagcctccttattactcctgccagtgtgccccaccattctcgggtagccgctgtgaactctacacggcaccccccagcacccctcctgccacctgtctgagccagtattgtgccgacaaagctcgggatggcgtctgtgatgaggcctgcaacagccatgcctgccagtgggatgggggtgactgttctctcaccatggagaacccctgggccaactgctcctccccacttccctgctgggattatatcaacaaccagtgtgatgagctgtgcaacacggtcgagtgcctgtttgacaactttgaatgccaggggaacagcaagacatgcaagtatgacaaatactgtgcagaccacttcaaagacaaccactgtaaccaggggtgcaacagtgaggagtgtggttgggatgggctggactgtgctgctgaccaacctgagaacctggcagaaggtaccctggttattgtggtattgatgccacctgaacaactgctccaggatgctcgcagcttcttgcgggcactgggtaccctgctccacaccaacctgcgcattaagcgggactcccagggggaactcatggtgtacccctattatggtgagaagtcagctgctatgaagaaacagaggatgacacgcagatcccttcctggtgaacaagaacaggaggtggctggctctaaagtctttctggaaattgacaaccgccagtgtgttcaagactcagaccactgcttcaagaacacggatgcagcagcagctctcctggcctctcacgccatacaggggaccctgtcataccctcttgtgtctgtcgtcagtgaatccctgactccagaacgcactcagctcctctatctccttgctgttgctgttgtcatcattctgtttattattctgctgggggtaatcatggcaaaacgaaagcgtaagcatggctctctctggctgcctgaaggtttcactcttcgccgagatgcaagcaatcacaagcgtcgtgagccagtgggacaggatgctgtggggctgaaaaatctctcagtgcaagtctcagaagctaacctaattggtactggaacaagtgaacactgggtcgatgatgaagggccccagccaaagaaagtaaaggctgaagatgaggccttactctcagaagaagatgaccccattgatcgacggccatggacacagcagcaccttgaagctgcagacatccgtaggacaccatcgctggctctcacccctcctcaggcagagcaggaggtggatgtgttagatgtgaatgtccgtggcccagatggctgcaccccattgatgttggcttctctccgaggaggcagctcagatttgagtgatgaagatgaagatgcagaggactcttctgctaacatcatcacagacttggtctaccagggtgccagcctccaggcccagacagaccggactggtgagatggccctgcaccttgcagcccgctactcacgggctgatgctgccaagcgtctcctggatgcaggtgcagatgccaatgcccaggacaacatgggccgctgtccactccatgctgcagtggcagctgatgcccaaggtgtcttccagattctgattcgcaaccgagtaactgatctagatgccaggatgaatgatggtactacacccctgatcctggctgcccgcctggctgtggagggaatggtggcagaactgatcaactgccaagcggatgtgaatgcagtggatgaccatggaaaatctgctcttcactgggcagctgctgtcaataatgtggaggcaactcttttgttgttgaaaaatggggccaaccgagacatgcaggacaacaaggaagagacacctctgtttcttgctgcccgggaggggagctatgaagcagccaagatcctgttagaccattttgccaatcgagacatcacagaccatatggatcgtcttccccgggatgtggctcgggatcgcatgcaccatgacattgtgcgccttctggatgaatacaatgtgaccccaagccctccaggcaccgtgttgacttctgctctctcacctgtcatctgtgggcccaacagatctttcctcagcctgaagcacaccccaatgggcaagaagtctagacggcccagtgccaagagtaccatgcctactagcctccctaaccttgccaaggaggcaaaggatgccaagggtagtaggaggaagaagtctctgagtgagaaggtccaactgtctgagagttcagtaactttatcccctgttgattccctagaatctcctcacacgtatgtttccgacaccacatcctctccaatgattacatcccctgggatcttacaggcctcacccaaccctatgttggccactgccgcccctcctgccccagtccatgcccagcatgcactatctttttctaaccttcatgaaatgcagcctttggcacatggggccagcactgtgcttccctcagtgagccagttgctatcccaccaccacattgtgtctccaggcagtggcagtgctggaagcttgagtaggctccatccagtcccagtcccagcagattggatgaaccgcatggaggtgaatgagacccagtacaatgagatgtttggtatggtcctggctccagctgagggcacccatcctggcatagctccccagagcaggccacctgaagggaagcacataaccacccctcgggagcccttgccccccattgtgactttccagctcatccctaaaggcagtattgcccaaccagcgggggctccccagcctcagtccacctgccctccagctgttgcgggccccctgcccaccatgtaccagattccagaaatggcccgtttgcccagtgtggctttccccactgccatgatgccccagcaggacgggcaggtagctcagaccattctcccagcctatcatcctttcccagcctctgtgggcaagtaccccacacccccttcacagcacagttatgcttcctcaaatgctgctgagcgaacacccagtcacagtggtcacctccagggtgagcatccctacctgacaccatccccagagtctcctgaccagtggtcaagttcatcaccccactctgcttctgactggtcagatgtgaccaccagccctacccctgggggtgctggaggaggtcagcggggacctgggacacacatgtctgagccaccacacaacaacatgcaggtttatgcgtgagagagtccacctccagtgtagagacataactgacttttgtaaatgctgctgaggaacaaatgaaggtcatccgggagagaaatgaagaaatctctggagccagcttctagaggtaggaaagagaagatgttcttattcagataatgcaagagaagcaattcgtcagtttcactgggtatctgcaaggcttattgattattctaatctaataagacaagtttgtggaaatgcaagatgaatacaagccttgggtccatgtttactctcttctatttggagaataagatggatgcttattgaagcccagacattcttgcagcttggactgcattttaagccctgcaggcttctgccatatccatgagaagattctacactagcgtcctgttgggaattatgccctggaattctgcctgaattgacctacgcatctcctcctccttggacattcttttgtcttcatttggtgcttttggttttgcacctctccgtgattgtagccctaccagcatgttatagggcaagacctttgtgcttttgatcattctggcccatgaaagcaactttggtctcctttcccctcctgtcttcccggtatcccttggagtctcacaaggtttactttggtatggttctcagcacaaacctttcaagtatgttgtttctttggaaaatggacatactgtattgtgttctcctgcatatatcattcctggagagagaaggggagaagaatacttttcttcaacaaattttgggggcaggagatcccttcaagaggctgcaccttaatttttcttgtctgtgtgcaggtcttcatataaactttaccaggaagaagggtgtgagtttgttgtttttctgtgtatgggcctggtcagtgtaaagttttatccttgatagtctagttactatgaccctccccacttttttaaaaccagaaaaaggtttggaatgttggaatgaccaagagacaagttaactcgtgcaagagccagttacccacccacaggtccccctacttcctgccaagcattccattgactgcctgtatggaacacatttgtcccagatctgagcattctaggcctgtttcactcactcacccagcatatgaaactagtcttaactgttgagcctttcctttcatatccacagaagacactgtctcaaatgttgtacccttgccatttaggactgaactttccttagcccaagggacccagtgacagttgtcttccgtttgtcagatgatcagtctctactgattatcttgctgcttaaaggcctgctcaccaatctttctttcacaccgtgtggtccgtgttactggtatacccagtatgttctcactgaagacatggactttatatgttcaagtgcaggaattggaaagttggacttgttttctatgatccaaaacagccctataagaaggttggaaaaggaggaactatatagcagcctttgctattttctgctaccatttcttttcctctgaagcggccatgacattccctttggcaactaacgtagaaactcaacagaacattttcctttcctagagtcaccttttagatgataatggacaactatagacttgctcattgttcagactgattgcccctcacctgaatccactctctgtattcatgctcttggcaatttctttgactttcttttaagggcagaagcattttagttaattgtagataaagaatagttttcttcctcttctccttgggccagttaataattggtccatggctacactgcaacttccgtccagtgctgtgatgcccatgacacctgcaaaataagttctgcctgggcattttgtagatattaacaggtgaattcccgactcttttggtttgaatgacagttctcattccttctatggctgcaagtatgcatcagtgcttcccacttacctgatttgtctgtcggtggccccatatggaaaccctgcgtgtctgttggcataatagtttacaaatggttttttcagtcctatccaaatttattgaaccaacaaaaataattacttctgccctgagataagcagattaagtttgttcattctctgctttattctctccatgtggcaacattctgtcagcctctttcatagtgtgcaaacattttatcattctaaatggtgactctctgcccttggacccatttattattcacagatggggagaacctatctgcatggaccctcaccatcctctgtgcagcacacacagtgcagggagccagtggcgatggcgatgactttcttcccctgg

SEQ ID NO: 6 - protein, 2471 aa, Homo sapiens
MPALRPALLWALLALWLCCAAPAHALQCRDGYEPCVNEGMCVTYHNGTGYCKCPEGFLGEYCQHRDPCEKNRCQNGGTCVAQAMLGKATCRCASGFTGEDCQYSTSHPCFVSRPCLNGGTCHMLSRDTYECTCQVGFTGKECQWTDACLSHPCANGSTCTTVANQFSCKCLTGFTGQKCETDVNECDIPGHCQHGGTCLNLPGSYQCQCPQGFTGQYCDSLYVPCAPSPCVNGGTCRQTGDFTFECNCLPGFEGSTCERNIDDCPNHRCQNGGVCVDGVNTYNCRCPPQWTGQFCTEDVDECLLQPNACQNGGTCANRNGGYGCVCVNGWSGDDCSENIDDCAFASCTPGSTCIDRVASFSCMCPEGKAGLLCHLDDACISNPCHKGALCDTNPLNGQYICTCPQGYKGADCTEDVDECAMANSNPCEHAGKCVNTDGAFHCECLKGYAGPRCEMDINECHSDPCQNDATCLDKIGGFTCLCMPGFKGVHCELEINECQSNPCVNNGQCVDKVNRFQCLCPPGFTGPVCQIDIDDCSSTPCLNGAKCIDHPNGYECQCATGFTGVLCEENIDNCDPDPCHHGQCQDGIDSYTCICNPGYMGAICSDQIDECYSSPCLNDGRCIDLVNGYQCNCQPGTSGVNCEINFDDCASNPCIHGICMDGINRYSCVCSPGFTGQRCNIDIDECASNPCRKGATCINGVNGFRCICPEGPHHPSCYSQVNECLSNPCIHGNCTGGLSGYKCLCDAGWVGINCEVDKNECLSNPCQNGGTCDNLVNGYRCTCKKGFKGYNCQVNIDECASNPCLNQGTCFDDISGYTCHCVLPYTGKNCQTVLAPCSPNPCENAAVCKESPNFESYTCLCAPGWQGQRCTIDIDECISKPCMNHGLCHNTQGSYMCECPPGFSGMDCEEDIDDCLANPCQNGGSCMDGVNTFSCLCLPGFTGDKCQTDMNECLSEPCKNGGTCSDYVNSYTCKCQAGFDGVHCENNINECTESSCFNGGTCVDGINSFSCLCPVGFTGSFCLHEINECSSHPCLNEGTCVDGLGTYRCSCPLGYTGKNCQTLVNLCSRSPCKNKGTCVQKKAESQCLCPSGWAGAYCDVPNVSCDIAASRRGVLVEHLCQHSGVCINAGNTHYCQCPLGYTGSYCEEQLDECASNPCQHGATCSDFIGGYRCECVPGYQGVNCEYEVDECQNQPCQNGGTCIDLVNHFKCSCPPGTRGLLCEENIDDCARGPHCLNGGQCMDRIGGYSCRCLPGFAGERCEGDINECLSNPCSSEGSLDCIQLTNDYLCVCRSAFTGRHCETFVDVCPQMPCLNGGTCAVASNMPDGFICRCPPGFSGARCQSSCGQVKCRKGEQCVHTASGPRCFCPSPRDCESGCASSPCQHGGSCHPQRQPPYYSCQCAPPFSGSRCELYTAPPSTPPATCLSQYCADKARDGVCDEACNSHACQWDGGDCSLTMENPWANCSSPLPCWDYINNQCDELCNTVECLFDNFECQGNSKTCKYDKYCADHFKDNHCDQGCNSEECGWDGLDCAADQPENLAEGTLVIVVLMPPEQLLQDARSFLRALGTLLHTNLRIKRDSQGELMVYPYYGEKSAAMKKQRMTRRSLPGEQEQEVAGSKVFLEIDNRQCVQDSDHCFKNTDAAAALLASHAIQGTLSYPLVSVVSESLTPERTQLLYLLAVAVVIILFIILLGVIMAKRKRKHGSLWLPEGFTLRRDASNHKRREPVGQDAVGLKNLSVQVSEANLIGTGTSEHWVDDEGPQPKKVKAEDEALLSEEDDPIDRRPWTQQHLEAADIRRTPSLALTPPQAEQEVDVLDVNVRGPDGCTPLMLASLRGGSSDLSDEDEDAEDSSANIITDLVYQGASLQAQTDRTGEMALHLAARYSRADAAKRLLDAGADANAQDNMGRCPLHAAVAADAQGVFQILIRNRVTDLDARMNDGTTPLILAARLAVEGMVAELINCQADVNAVDDHGKSALHWAAAVNNVEATLLLLKNGANRDMQDNKEETPLFLAAREGSYEAAKILLDHFANRDITDHMDRLPRDVARDRMHHDIVRLLDEYNVTPSPPGTVLTSALSPVICGPNRSFLSLKHTPMGKKSRRPSAKSTMPTSLPNLAKEAKDAKGSRRKKSLSEKVQLSESSVTLSPVDSLESPHTYVSDTTSSPMITSPGILQASPNPMLATAAPPAPVHAQHALSFSNLHEMQPLAHGASTVLPSVSQLLSHHHIVSPGSGSAGSLSRLHPVPVPADWMNRMEVNETQYNEMFGMVLAPAEGTHPGIAPQSRPPEGKHITTPREPLPPIVTFQLIPKGSIAQPAGAPQPQSTCPPAVAGPLPTMYQIPEMARLPSVAFPTAMMPQQDGQVAQTILPAYHPFPASVGKYPTPPSQHSYASSNAAERTPSHSGHLQGEHPYLTPSPESPDQWSSSSPHSASDWSDVTTSPTPGGAGGGQRGPGTHMSEPPHNNMQVYA

SEQ ID NO: 7 - DNA, 8091 bp, Homo sapiens
acgcggcgcggaggctggcccgggacgcgcccggagcccagggaaggagggaggaggggagggtcgcggccggccgccatggggccgggggcccgtggccgccgccgccgccgtcgcccgatgtcgccgccaccgccaccgccacccgtgcgggcgctgcccctgctgctgctgctagcggggccgggggctgcagcccccccttgcctggacggaagcccgtgtgcaaatggaggtcgttgcacccagctgccctcccgggaggctgcctgcctgtgcccgcctggctgggtgggtgagcggtgtcagctggaggacccctgtcactcaggcccctgtgctggccgtggtgtctgccagagttcagtggtggctggcaccgcccgattctcatgccggtgcccccgtggcttccgaggccctgactgctccctgccagatccctgcctcagcagcccttgtgcccacggtgcccgctgctcagtggggcccgatggacgcttcctctgctcctgcccacctggctaccagggccgcagctgccgaagcgacgtggatgagtgccgggtgggtgagccctgccgccatggtggcacctgcctcaacacacctggctccttccgctgccagtgtccagctggctacacagggccactatgtgagaaccccgcggtgccctgtgcgccctcaccatgccgtaacgggggcacctgcaggcagagtggcgacctcacttacgactgtgcctgtcttcctgggtttgagggtcagaattgtgaagtgaacgtggacgactgtccaggacaccgatgtctcaatggggggacatgcgtggatggcgtcaacacctataactgccagtgccctcctgagtggacaggccagttctgcacggaggacgtggatgagtgtcagctgcagcccaacgcctgccacaatgggggtacctgcttcaacacgctgggtggccacagctgcgtgtgtgtcaatggctggacaggtgagagctgcagtcagaatatcgatgactgtgccacagccgtgtgcttccatggggccacctgccatgaccgcgtggcttctttctactgtgcctgccccatgggcaagactggcctcctgtgtcacctggatgacgcctgtgtcagcaacccctgccacgaggatgctatctgtgacacaaatccggtgaacggccgggccatttgcacctgtcctcccggcttcacgggtggggcatgtgaccaggatgtggacgagtgctctatcggcgccaacccctgcgagcacttgggcaggtgcgtgaacacgcagggctccttcctgtgccagtgcggtcgtggctacactggacctcgctgtgagaccgatgtcaacgagtgtctgtcggggccctgccgaaaccaggccacgtgcctcgaccgcataggccagttcacctgtatctgtatggcaggcttcacaggaacctattgcgaggtggacattgacgagtgtcagagtagcccctgtgtcaacggtggggtctgcaaggaccgagtcaatggcttcagctgcacctgcccctcgggcttcagcggctccacgtgtcagctggacgtggacgaatgcgccagcacgccctgcaggaatggcgccaaatgcgtggaccagcccgatggctacgagtgccgctgtgccgagggctttgagggcacgctgtgtgatcgcaacgtggacgactgctcccctgacccatgccaccatggtcgctgcgtggatggcatcgccagcttctcatgtgcctgtgctcctggctacacgggcacacgctgcgagagccaggtggacgaatgccgcagccagccctgccgccatggcggcaaatgcctagacctggtggacaagtacctctgccgctgcccttctgggaccacaggtgtgaactgcgaagtgaacattgacgactgtgccagcaacccctgcacctttggagtctgccgtgatggcatcaaccgctacgactgtgtctgccaacctggcttcacagggcccctttgtaacgtggagatcaatgagtgtgcttccagcccatgcggcgagggaggttcctgtgtggatggggaaaatggcttccgctgcctctgcccgcctggctccttgcccccactctgcctccccccgagccatccctgtgcccatgagccctgcagtcacggcatctgctatgatgcacctggcgggttccgctgtgtgtgtgagcctggctggagtggcccccgctgcagccagagcctggcccgagacgcctgtgagtcccagccgtgcagggccggtgggacatgcagcagcgatggaatgggtttccactgcacctgcccgcctggtgtccagggacgtcagtgtgaactcctctccccctgcaccccgaacccctgtgagcatgggggccgctgcgagtctgcccctggccagctgcctgtctgctcctgcccccagggctggcaaggcccacgatgccagcaggatgtggacgagtgtgctggccccgcaccctgtggccctcatggtatctgcaccaacctggcagggagtttcagctgcacctgccatggagggtacactggcccttcctgtgatcaggacatcaatgactgtgaccccaacccatgcctgaacggtggctcgtgccaagacggcgtgggctccttttcctgctcctgcctccctggtttcgccggcccacgatgcgcccgcgatgtggatgagtgcctgagcaacccctgcggcccgggcacctgtaccgaccacgtggcctccttcacctgcacctgcccgccgggctacggaggcttccactgcgaacaggacctgcccgactgcagccccagctcctgcttcaatggcgggacctgtgtggacggcgtgaactcgttcagctgcctgtgccgtcccggctacacaggagcccactgccaacatgaggcagacccctgcctctcgcggccctgcctacacgggggcgtctgcagcgccgcccaccctggcttccgctgcacctgcctcgagagcttcacgggcccgcagtgccagacgctggtggattggtgcagccgccagccttgtcaaaacgggggtcgctgcgtccagactggggcctattgcctttgtccccctggatggagcggacgcctctgtgacatccgaagcttgccctgcagggaggccgcagcccagatcggggtgcggctggagcagctgtgtcaggcgggtgggcagtgtgtggatgaagacagctcccactactgcgtgtgcccagagggccgtactggtagccactgtgagcaggaggtggacccctgcttggcccagccctgccagcatggggggacctgccgtggctatatggggggctacatgtgtgagtgtcttcctggctacaatggtgataactgtgaggacgacgtggacgagtgtgcctcccagccctgccagcacgggggttcatgcattgacctcgtggcccgctatctctgctcctgtcccccaggaacgctgggggtgctctgcgagattaatgaggatgactgcggcccaggcccaccgctggactcagggccccggtgcctacacaatggcacctgcgtggacctggtgggtggtttccgctgcacctgtcccccaggatacactggtttgcgctgcgaggcagacatcaatgagtgtcgctcaggtgcctgccacgcggcacacacccgggactgcctgcaggacccaggcggaggtttccgttgcctttgtcatgctggcttctcaggtcctcgctgtcagactgtcctgtctccctgcgagtcccagccatgccagcatggaggccagtgccgtcctagcccgggtcctgggggtgggctgaccttcacctgtcactgtgcccagccgttctggggtccgcgttgcgagcgggtggcgcgctcctgccgggagctgcagtgcccggtgggcgtcccatgccagcagacgccccgcgggccgcgctgcgcctgccccccagggttgtcgggaccctcctgccgcagcttcccggggtcgccgccgggggccagcaacgccagctgcgcggccgccccctgtctccacgggggctcctgccgccccgcgccgctcgcgcccttcttccgctgcgcttgcgcgcagggctggaccgggccgcgctgcgaggcgcccgccgcggcacccgaggtctcggaggagccgcggtgcccgcgcgccgcctgccaggccaagcgcggggaccagcgctgcgaccgcgagtgcaacagcccaggctgcggctgggacggcggcgactgctcgctgagcgtgggcgacccctggcggcaatgcgaggcgctgcagtgctggcgcctcttcaacaacagccgctgcgaccccgcctgcagctcgcccgcctgcctctacgacaacttcgactgccacgccggtggccgcgagcgcacttgcaacccggtgtacgagaagtactgcgccgaccactttgccgacggccgctgcgaccagggctgcaacacggaggagtgcggctgggatgggctggattgtgccagcgaggtgccggccctgctggcccgcggcgtgctggtgctcacagtgctgctgccgccggaggagctactgcgttccagcgccgactttctgcagcggctcagcgccatcctgcgcacctcgctgcgcttccgcctggacgcgcacggccaggccatggtcttcccttaccaccggcctagtcctggctccgaaccccgggcccgtcgggagctggcccccgaggtgatcggctcggtagtaatgctggagattgacaaccggctctgcctgcagtcgcctgagaatgatcactgcttccccgatgcccagagcgccgctgactacctgggagcgttgtcagcggtggagcgcctggacttcccgtacccactgcgggacgtgcggggggagccgctggagcctccagaacccagcgtcccgctgctgccactgctagtggcgggcgctgtcttgctgctggtcattctcgtcctgggtgtcatggtggcccggcgcaagcgcgagcacagcaccctctggttccctgagggcttctcactgcacaaggacgtggcctctggtcacaagggccggcgggaacccgtgggccaggacgcgctgggcatgaagaacatggccaagggtgagagcctgatgggggaggtggccacagactggatggacacagagtgcccagaggccaagcggctaaaggtagaggagccaggcatgggggctgaggaggctgtggattgccgtcagtggactcaacaccatctggttgctgctgacatccgcgtggcaccagccatggcactgacaccaccacagggcgacgcagatgctgatggcatggatgtcaatgtgcgtggcccagatggcttcaccccgctaatgctggcttccttctgtgggggggctctggagccaatgccaactgaagaggatgaggcagatgacacatcagctagcatcatctccgacctgatctgccagggggctcagcttggggcacggactgaccgtactggcgagactgctttgcacctggctgcccgttatgcccgtgctgatgcagccaagcggctgctggatgctggggcagacaccaatgcccaggaccactcaggccgcactcccctgcacacagctgtcacagccgatgcccagggtgtcttccagattctcatccgaaaccgctctacagacttggatgcccgcatggcagatggctcaacggcactgatcctggcggcccgcctggcagtagagggcatggtggaagagctcatcgccagccatgctgatgtcaatgctgtggatgagcttgggaaatcagccttacactgggctgcggctgtgaacaacgtggaagccactttggccctgctcaaaaatggagccaataaggacatgcaggatagcaaggaggagacccccctattcctggccgcccgcgagggcagctatgaggctgccaagctgctgttggaccactttgccaaccgtgagatcaccgaccacctggacaggctgccgcgggacgtagcccaggagagactgcaccaggacatcgtgcgcttgctggatcaacccagtgggccccgcagcccccccggtccccacggcctggggcctctgctctgtcctccaggggccttcctccctggcctcaaagcggcacagtcggggtccaagaagagcaggaggccccccgggaaggcggggctggggccgcaggggccccgggggcggggcaagaagctgacgctggcctgcccgggccccctggctgacagctcggtcacgctgtcgcccgtggactcgctggactccccgcggcctttcggtgggccccctgcttcccctggtggcttcccccttgaggggccctatgcagctgccactgccactgcagtgtctctggcacagcttggtggcccaggccgggcaggtctagggcgccagccccctggaggatgtgtactcagcctgggcctgctgaaccctgtggctgtgcccctcgattgggcccggctgcccccacctgcccctccaggcccctcgttcctgctgccactggcgccgggaccccagctgctcaacccagggacccccgtctccccgcaggagcggcccccgccttacctggcagtcccaggacatggcgaggagtacccggtggctggggcacacagcagccccccaaaggcccgcttcctgcgggttcccagtgagcacccttacctgaccccatcccccgaatcccctgagcactgggccagcccctcacctccctccctctcagactggtccgaatccacgcctagcccagccactgccactggggccatggccaccaccactggggcactgcctgcccagccacttcccttgtctgttcccagctcccttgctcaggcccagacccagctggggccccagccggaagttacccccaagaggcaagtgttggcctgagacgctcgtcagttcttagatcttgggggcctaaagagacccccgtcctgcctcctttctttctctgtctcttccttccttttagtctttttcatcctcttctctttccaccaaccctcctgcatccttgccttgcagcgtgaccgagataggtcatcagcccagggcttcagtcttcctttatttataatgggtgggggctaccacccaccctctcagtcttgtgaagagtctgggacctccttcttccccacttctctcttccctcattcctttctctctccttctggcctctcatttccttacactctgacatgaatgaattattattatttttctttttctttttttttttacattttgtatagaaacaaattcatttaaacaaacttattattattattttttacaaaatatatatatggagatgctccctccccctgtgaaccccccagtgcccccgtggggctgagtctgtgggcccattcggccaagctggattctgtgtacctagtacacaggcatgactgggatcccgtgtaccgagtacacgacccaggtatgtaccaagtaggcacccttgggcgcacccactggggccaggggtcgggggagtgttgggagcctcctccccaccccacctccctcacttcactgcattccagattggacatgttccatagccttgctggggaagggcccactgccaactccctctgccccagccccacccttggccatctccctttgggaactagggggctgctggtgggaaatgggagccagggcagatgtatgcattcctttatgtccctgtaaatgtgggactacaagaagaggagctgcctgagtggtactttctcttcctggtaatcctctggcccagccttatggcagaatagaggtatttttaggctatttttgtaatatggcttctggtcaaaatccctgtgtagctgaattcccaagccctgcattgtacagccccccactcccctcaccacctaataaaggaatagttaacactcaaaaaaaaaaaaaaaaaaa

SEQ ID NO: 8 - protein, 2321 aa, Homo sapiens
MGPGARGRRRRRRPMSPPPPPPPVRALPLLLLLAGPGAAAPPCLDGSPCANGGRCTQLPSREAACLCPPGWVGERCQLEDPCHSGPCAGRGVCQSSVVAGTARFSCRCPRGFRGPDCSLPDPCLSSPCAHGARCSVGPDGRFLCSCPPGYQGRSCRSDVDECRVGEPCRHGGTCLNTPGSFRCQCPAGYTGPLCENPAVPCAPSPCRNGGTCRQSGDLTYDCACLPGFEGQNCEVNVDDCPGHRCLNGGTCVDGVNTYNCQCPPEWTGQFCTEDVDECQLQPNACHNGGTCFNTLGGHSCVCVNGWTGESCSQNIDDCATAVCFHGATCHDRVASFYCACPMGKTGLLCHLDDACVSNPCHEDAICDTNPVNGRAICTCPPGFTGGACDQDVDECSIGANPCEHLGRCVNTQGSFLCQCGRGYTGPRCETDVNECLSGPCRNQATCLDRIGQFTCICMAGFTGTYCEVDIDECQSSPCVNGGVCKDRVNGFSCTCPSGFSGSTCQLDVDECASTPCRNGAKCVDQPDGYECRCAEGFEGTLCDRNVDDCSPDPCHHGRCVDGIASFSCACAPGYTGTRCESQVDECRSQPCRHGGKCLDLVDKYLCRCPSGTTGVNCEVNIDDCASNPCTFGVCRDGINRYDCVCQPGFTGPLCNVEINECASSPCGEGGSCVDGENGFRCLCPPGSLPPLCLPPSHPCAHEPCSHGICYDAPGGFRCVCEPGWSGPRCSQSLARDACESQPCRAGGTCSSDGMGFHCTCPPGVQGRQCELLSPCTPNPCEHGGRCESAPGQLPVCSCPQGWQGPRCQQDVDECAGPAPCGPHGICTNLAGSFSCTCHGGYTGPSCDQDINDCDPNPCLNGGSCQDGVGSFSCSCLPGFAGPRCARDVDECLSNPCGPGTCTDHVASFTCTCPPGYGGFHCEQDLPDCSPSSCFNGGTCVDGVNSFSCLCRPGYTGAHCQHEADPCLSRPCLHGGVCSAAHPGFRCTCLESFTGPQCQTLVDWCSRQPCQNGGRCVQTGAYCLCPPGWSGRLCDIRSLPCREAAAQIGVRLEQLCQAGGQCVDEDSSHYCVCPEGRTGSHCEQEVDPCLAQPCQHGGTCRGYMGGYMCECLPGYNGDNCEDDVDECASQPCQHGGSCIDLVARYLCSCPPGTLGVLCEINEDDCGPGPPLDSGPRCLHNGTCVDLVGGFRCTCPPGYTGLRCEADINECRSGACHAAHTRDCLQDPGGGFRCLCHAGFSGPRCQTVLSPCESQPCQHGGQCRPSPGPGGGLTFTCHCAQPFWGPRCERVARSCRELQCPVGVPCQQTPRGPRCACPPGLSGPSCRSFPGSPPGASNASCAAAPCLHGGSCRPAPLAPFFRCACAQGWTGPRCEAPAAAPEVSEEPRCPRAACQAKRGDQRCDRECNSPGCGWDGGDCSLSVGDPWRQCEALQCWRLFNNSRCDPACSSPACLYDNFDCHAGGRERTCNPVYEKYCADHFADGRCDQGCNTEECGWDGLDCASEVPALLARGVLVLTVLLPPEELLRSSADFLQRLSAILRTSLRFRLDAHGQAMVFPYHRPSPGSEPRARRELAPEVIGSVVMLEIDNRLCLQSPENDHCFPDAQSAADYLGALSAVERLDFPYPLRDVRGEPLEPPEPSVPLLPLLVAGAVLLLVILVLGVMVARRKREHSTLWFPEGFSLHKDVASGHKGRREPVGQDALGMKNMAKGESLMGEVATDWMDTECPEAKRLKVEEPGMGAEEAVDCRQWTQHHLVAADIRVAPAMALTPPQGDADADGMDVNVRGPDGFTPLMLASFCGGALEPMPTEEDEADDTSASIISDLICQGAQLGARTDRTGETALHLAARYARADAAKRLLDAGADTNAQDHSGRTPLHTAVTADAQGVFQILIRNRSTDLDARMADGSTALILAARLAVEGMVEELIASHADVNAVDELGKSALHWAAAVNNVEATLALLKNGANKDMQDSKEETPLFLAAREGSYEAAKLLLDHFANREITDHLDRLPRDVAQERLHQDIVRLLDQPSGPRSPPGPHGLGPLLCPPGAFLPGLKAAQSGSKKSRRPPGKAGLGPQGPRGRGKKLTLACPGPLADSSVTLSPVDSLDSPRPFGGPPASPGGFPLEGPYAAATATAVSLAQLGGPGRAGLGRQPPGGCVLSLGLLNPVAVPLDWARLPPPAPPGPSFLLPLAPGPQLLNPGTPVSPQERPPPYLAVPGHGEEYPAAGAHSSPPKARFLRVPSEHPYLTPSPESPEHWASPSPPSLSDWSESTPSPATATGAMATTTGALPAQPLPLSVPSSLAQAQTQLGPQPEVTPKRQVLA

SEQ ID NO: 9 - DNA, 3877 bp, Homo sapiens
gacagcggcccctcctatttctgccactgcccccctggattccaaggcagcctgtgccaggatcacgtgaacccatgtgagtccaggccttgccagaacggggccacctgcatggcccagcccagtgggtatctctgccagtgtgccccaggctacgatggacagaactgctcaaaggaactcgatgcttgtcagtcccaaccctgtcacaaccatggaacctgtactcccaaacctggaggcttccactgtgcctgccctccaggctttgtggggctacgctgtgagggagacgtggacgagtgtctggaccagccctgccaccccacaggcactgcagcctgccactctctggccaatgccttctactgccagtgtctgcctggacacacaggccagtggtgtgaggtggagatagacccctgccacagccaaccctgctttcatggagggacctgtgaggccacagcaggatcacccctgggtttcatctgccactgccccaagggttttgaaggccccacctgcagccacagggccccttcctgcggcttccatcactgccaccacggaggcctgtgtctgccctcccctaagccaggcttcccaccacgctgtgcctgcctcagtggctatgggggtcctgactgcctgaccccaccagctcctaaaggctgtggccctccctccccatgcctatacaatggcagctgctcagagaccacgggcttggggggcccaggctttcgatgctcctgccctcacagctctccagggccccggtgtcagaaacccggagccaaggggtgtgagggcagaagtggagatggggcctgcgatgctggctgcagtggcccgggaggaaactgggatggaggggactgctctctgggagtcccagacccctggaagggctgcccctcccactctcggtgctggcttctcttccgggacgggcagtgccacccacagtgtgactctgaagagtgtctgtttgatggctacgactgtgagacccctccagcctgcactccagcctatgaccagtactgccatgatcacttccacaacgggcactgtgagaaaggctgcaacactgcagagtgtggctgggatggaggtgactgcaggcctgaagatggggacccagagtgggggccctccctggccctgctggtggtactgagccccccagccctagaccagcagctgtttgccctggcccgggtgctgtccctgactctgagggtaggactctgggtaaggaaggatcgtgatggcagggacatggtgtacccctatcctggggcccgggctgaagaaaagctaggaggaactcgggaccccacctatcaggagagagcagcccctcaaacgcagcccctgggcaaggagaccgactccctcagtgctgggtttgtggtggtcatgggtgtggatttgtcccgctgtggccctgaccacccggcatcccgctgtccctgggaccctgggcttctactccgcttccttgctgcgatggctgcagtgggagccctggagcccctgctgcctggaccactgctggttgtccaccctcatgaaggggaccgaaccccctgccaaccagtttccctggcctgtgttgtgctccccagtggccggggtgattctcctggccctaggggtcttctcgtcctccagctcatccggcgtcgacgccgagagcatggagctctctggctgccccctggtttcactcgacggcctcggactcagtcagctccccaccgacgccggcccccactaggcgaggacagcattggtctcaaggcactgaagccaaaggcagaagttgatgaggatggagttgtgatgtgctcaggccctgaggagggagaggaggtggcccaggctgaagaaacaggcccaccctccacgtgccagctctggtctctgagtggtggctgtggggcgctccctcaggcagccatgctaactcctccccaggaatctgagatggaagcccctgacctggacacccgtggacctgatggggtgacacccctgatgtcagcagtttgctgtggggaagtacagtccgggaccttccaaggggcatggttgggatgtcctgagccctgggaacctctgctggatggaggggcctgtccccaggctcacaccgtgggcactggggagacccccctgcacctggctgcccgattctcccggccaaccgctgcccgccgcctccttgaggctggagccaaccccaaccagccagaccgggcagggcgcacaccccttcatgctgctgtggctgctgatgctcgggaggtctgccagcttctgctccgtagcagacaaactgcagtggacgctcgcacagaggacgggaccacacccttgatgctggctgccaggctggcggtggaagacctggttgaagaactgattgcagcccaagcagacgtgggggccagagataaatgggggaaaactgcgctgcactgggctgctgccgtgaacaacgcccgagccgcccgctcgcttctccaggccggagccgataaagatgcccaggacaacagggagcagacgccgctattcctggcggcgcgggaaggagcggtggaagtagcccagctactgctggggctgggggcagcccgagagctgcgggaccaggctgggctagcgccggcggacgtcgctcaccaacgtaaccactgggatctgctgacgctgctggaaggggctgggccaccagaggcccgtcacaaagccacgccgggccgcgaggctgggcccttcccgcgcgcacggacggtgtcagtaagcgtgcccccgcatgggggcggggctctgccgcgctgccggacgctgtcagccggagcaggccctcgtgggggcggagcttgtctgcaggctcggacttggtccgtagacttggctgcgcgggggggcggggcctattctcattgccggagcctctcgggagtaggagcaggaggaggcccgacccctcgcggccgtaggttttctgcaggcatgcgcgggcctcggcccaaccctgcgataatgcgaggaagatacggagtggctgccgggcgcggaggcagggtctcaacggatgactggccctgtgattgggtggccctgggagcttgcggttctgcctccaacattccgatcccgcctccttgccttactccgtccccggagcggggatcacctcaacttgactgtggtcccccagccctccaagaaatgcccataaaccaaggaggagagggtaaaaaatagaagaatacatggtagggaggaattccaaaaatgattacccattaaaaggcaggctggaaggccttcctggttttaagatggatcccccaaaatgaagggttgtgagtttagtttctctcctaaaatgaatgtatgcccaccagagcagacatcttccacgtggagaagctgcagctctggaaagagggtttaagatgctaggatgaggcaggcccagtcctcctccagaaaataagacaggccacaggagggcagagtggagtggaaatacccctaagttggaaccaagaattgcaggcatatgggatgtaagatgttctttcctatatatggtttccaaagggtgcccctatgatccattgtccccactgcccacaaatggctgacaaatatttattgggcacctactatgtgccaggcactgtgtaggtgctgaaaagtggccaagggccacccccgctgatgactccttgcattccctcccctcacaacaaagaactccactgtggggatgaagcgcttcttctagccactgctatcgctatttaagaaccctaaatctgtcacccataataaagctgatttgaag

SEQ ID NO: 10 - protein, 2003 aa, Homo sapiens
MQPPSLLLLLLLLLLLCVSVVRPRGLLCGSFPEPCANGGTCLSLSLGQGTCQCAPGFLGETCQFPDPCQNAQLCQNGGSCQALLPAPLGLPSSPSPLTPSFLCTCLPGFTGERCQAKLEDPCPPSFCSKRGRCHIQASGRPQCSCMPGWTGEQCQLRDFCSANPCVNGGVCLATYPQIQCHCPPGFEGHACERDVNECFQDPGPCPKGTSCHNTLGSFQCLCPVGQEGPRCELRAGPCPPRGCSNGGTCQLMPEKDSTFHLCLCPPGFIGPDCEVNPDNCVSHQCQNGGTCQDGLDTYTCLCPETWTGWDCSEDVDECETQGPPHCRNGGTCQNSAGSFHCVCVSGWGGTSCEENLDDCIAATCAPGSTCIDRVGSFSCLCPPGRTGLLCHLEDMCLSQPCHGDAQCSTNPLTGSTLCLCQPGYSGPTCHQDLDECLMAQQGPSPCEHGGSCLNTPGSFNCLCPPGYTGSRCEADHNECLSQPCHPGSTCLDLLATFHCLCPPGLEGQLCEVETNECASAPCLNHADCHDLLNGFQCICLPGFSGTRCEEDIDECRSSPCANGGQCQDQPGAFHCKCLPGFEGPRCQTEVDECLSDPCPVGASCLDLPGAFFCLCPSGFTGQLCEVPLCAPNLCQPKQICKDQKDKANCLCPDGSPGCAPPEDNCTCHHGHCQRSSCVCDVGWTGPECEAELGGCISAPCAHGGTCYPQPSGYNCTCPTGYTGPTCSEEMTACHSGPCLNGGSCNPSPGGYYCTCPPSHTGPQCQTSTDYCVSAPCFNGGTCVNRPGTFSCLCAMGFQGPRCEGKLRPSCADSPCRNRATCQDSPQGPRCLCPTGYTGGSCQTLMDLCAQKPCPRNSHCLQTGPSFHCLCLQGWTGPLCNLPLSSCQKAALSQGIDVSSLCHNGGLCVDSGPSYFCHCPPGFQGSLCQDHVNPCESRPCQNGATCMAQPSGYLCQCAPGYDGQNCSKELDACQSQPCHNHGTCTPKPGGFHCACPPGFVGLRCEGDVDECLDQPCHPTGTAACHSLANAFYCQCLPGHTGQWCEVEIDPCHSQPCFHGGTCEATAGSPLGFICHCPKGFEGPTCSHRAPSCGFHHCHHGGLCLPSPKPGFPPRCACLSGYGGPDCLTPPAPKGCGPPSPCLYNGSCSETTGLGGPGFRCSCPHSSPGPRCQKPGAKGCEGRSGDGACDAGCSGPGGNWDGGDCSLGVPDPWKGCPSHSRCWLLFRDGQCHPQCDSEECLFDGYDCETPPACTPAYDQYCHDHFHNGHCEKGCNTAECGWDGGDCRPEDGDPEWGPSLALLVVLSPPALDQQLFALARVLSLTLRVGLWVRKDRDGRDMVYPYPGARAEEKLGGTRDPTYQERAAPQTQPLGKETDSLSAGFVVVMGVDLSRCGPDHPASRCPWDPGLLLRFLAAMAAVGALEPLLPGPLLAVHPHAGTAPPANQLPWPVLCSPVAGVILLALGALLVLQLIRRRRREHGALWLPPGFTRRPRTQSAPHRRRPPLGEDSIGLKALKPKAEVDEDGVVMCSGPEEGEEVGQAEETGPPSTCQLWSLSGGCGALPQAAMLTPPQESEMEAPDLDTRGPDGVTPLMSAVCCGEVQSGTFQGAWLGCPEPWEPLLDGGACPQAHTVGTGETPLHLAARFSRPTAARRLLEAGANPNQPDRAGRTPLHAAVAADAREVCQLLLRSRQTAVDARTEDGTTPLMLAARLAVEDLVEELIAAQADVGARDKWGKTALHWAAAVNNARAARSLLQAGADKDAQDNREQTPLFLAAREGAVEVAQLLLGLGAARELRDQAGLAPADVAHQRNHWDLLTLLEGAGPPEARHKATPGREAGPFPRARTVSVSVPPHGGGALPRCRTLSAGAGPRGGGACLQARTWSVDLAARGGGAYSHCRSLSGVGAGGGPTPRGRRFSAGMRGPRPNPAIMRGRYGVAAGRGGRVSTDDWPCDWVALGACGSASNIPIPPPCLTPSPERGSPQLDCGPPALQEMPINQGGEGKK

SEQ ID NO: 11 - DNA, 3162 bp, Homo sapiens
gaattccattttaagttatacaaaactgattaccataagtgcggtcgactgcttttatttttacgttgtgtgtgttggaaaaatgctaaaacatcagtctacaattctatatattgttattaaagattaatccaaccagcaacccaaggacatataagcgatttccactattgcatcagagcactcggcaggaaaggcctagccacggggaacattagaagctacagaagcattgcagagaagagaagatcccccgcgcgtccgccgctgttctaaggagagaagtgggggccccccaggctcgcgcgtggagcgaagcagcatgggcagtcggtgcgcgctggccctggcggtgctctcggccttgctgtgtcaggtctggagctctggggtgttcgaactgaagctgcaggagttcgtcaacaagaaggggctgctggggaaccgcaactgctgccgcgggggcgcggggccaccgccgtgcgcctgccggaccttcttccgcgtgtgcctcaagcactaccaggccagcgtgtcccccgagccgccctgcacctacggcagcgccgtcacccccgtgctgggcgtcgactccttcagtctgcccgacggcgggggcgccgactccgcgttcagcaaccccatccgcttccccttcggcttcacctggccgggcaccttctctctgattattgaagctctccacacagattctcctgatgacctcgcaacagaaaacccagaaagactcatcagccgcctggccacccagaggcacctgacggtgggcgaggagtggtcccaggacctgcacagcagcggccgcacggacctcaagtactcctaccgcttcgtgtgtgacgaacactactacggagagggctgctccgttttctgccgtccccgggacgatgccttcggccacttcacctgtggggagcgtggggagaaagtgtgcaaccctggctggaaagggccctactgcacagagccgatctgcctgcctggatgtgatgagcagcatggattttgtgacaaaccaggggaatgcaagtgcagagtgggctggcagggccggtactgtgacgagtgtatccgctatccaggctgtctccatggcacctgccagcagccctggcagtgcaactgccaggaaggctgggggggccttttctgcaaccaggacctgaactactgcacacaccataagccctgcaagaatggagccacctgcaccaacacgggccaggggagctacacttgctcttgccggcctgggtacacaggtgccacctgcgagctggggattgacgagtgtgaccccagcccttgtaagaacggagggagctgcacggatctcgagaacagctactcctgtacctgcccacccggcttctacggcaaaatctgtgaattgagtgccatgacctgtgcggacggcccttgctttaacgggggtcggtgctcagacagccccgatggagggtacagctgccgctgccccgtgggctactccggcttcaactgtgagaagaaaattgactactgcagctcttcaccctgttctaatggtgccaagtgtgtggacctcggtgatgcctacctgtgccgctgccaggccggcttctcggggaggcactgtgacgacaacgtggacgactgcgcctcctccccgtgcgccaacgggggcacctgccgggatggcgtgaacgacttctcctgcacctgcccgcctggctacacgggcaggaactgcagtgcccccgtcagcaggtgcgagcacgcaccctgccacaatggggccacctgccacgagaggggccacggctatgtgtgcgagtgtgcccgaggctacgggggtcccaactgccagttcctgctccccgagctgcccccgggcccagcggtggtggacctcactgagaagctagagggccagggcgggccattcccctgggtggccgtgtgcgccggggtcatccttgtcctcatgctgctgctgggctgtgccgctgtggtggtctgcgtccggctgaggctgcagaagcaccggcccccagccgacccctgccggggggagacggagaccatgaacaacctggccaactgccagcgtgagaaggacatctcagtcagcatcatcggggccacgcagatcaagaacaccaacaagaaggcggacttccacggggaccacagcgccgacaagaatggcttcaaggcccgctacccagcggtggactataacctcgtgcaggacctcaagggtgacgacaccgccgtcagggacgcgcacagcaagcgtgacaccaagtgccagccccagggctcctcaggggaggagaaggggaccccgaccacactcaggggtggagaagcatctgaaagaaaaaggccggactcgggctgttcaacttcaaaagacaccaagtaccagtcggtgtacgtcatatccgaggagaaggatgagtgcgtcatagcaactgaggtgtaaaatggaagtgagatggcaagactcccgtttctcttaaaataagtaaaattccaaggatatatgccccaacgaatgctgctgaagaggagggaggcctcgtggactgctgctgagaaaccgagttcagaccgagcaggttctcctcctgaggtcctcgacgcctgccgacagcctgtcgcggcccggccgcctgcggcactgccttccgtgacgtcgccgttgcactatggacagttgctcttaagagaatatatatttaaatgggtgaactgaattacgcataagaagcatgcactgcctgagtgtatattttggattcttatgagccagtcttttcttgaattagaaacacaaacactgcctttattgtcctttttgatacgaagatgtgctttttctagatggaaaagatgtgtgttattttttggatttgtaaaaatatttttcatgatatctgtaaagcttgagtattttgtgatgttcgttttttataatttaaattttggtaaatatgtacaaaggcacttcgggtctatgtgactatatttttttgtatataaatgtatttatggaatattgtgcaaatgttatttgagttttttactgttttgttaatgaagaaattcctttttaaaatatttttccaaaataaattttatgaggaattc

SEQ ID NO: 12 - protein, 723 aa, Homo sapiens
MGSRCALALAVLSALLCQVWSSGVFELKLQEFVNKKGLLGNRNCCRGGAGPPPCACRTFFRVCLKHYQASVSPEPPCTYGSAVTPVLGVDSFSLPDGGGADSAFSNPIRFPFGFTWPGTFSLIIEALHTDSPDDLATENPERLISRLATQRHLTVGEEWSQDLHSSGRTDLKYSYRFVCDEHYYGEGCSVFCRPRDDAFGHFTCGERGEKVCNPGWKGPYCTEPICLPGCDEQHGFCDKPGECKCRVGWQGRYCDECIRYPGCLHGTCQQPWQCNCQEGWGGLFCNQDLNYCTHHKPCKNGATCTNTGQGSYTCSCRPGYTGATCELGIDECDPSPCKNGGSCTDLENSYSCTCPPGFYGKICELSAMTCADGPCFNGGRCSDSPDGGYSCRCPVGYSGFNCEKKIDYCSSSPCSNGAKCVDLGDAYLCRCQAGFSGRHCDDNVDDCASSPCANGGTCRDGVNDFSCTCPPGYTGRNCSAPVSRCEHAPCHNGATCHERGHRYVCECARGYGGPNCQFLLPELPPGPAVVDLTEKLEGQGGPFPWVAVCAGVILVLMLLLGCAAVVVCVRLRLQKHRPPADPCRGETETMNNLANCQREKDISVSIIGATQIKNTNKKADFHGDHSADKNGFKARYPAVDYNLVQDLKGDDTAVRDAHSKRDTKCQPQGSSGEEKGTPTTLRGGEASERKRPDSGCSTSKDTKYQSVYVISEEKDECVIATEV

SEQ ID NO: 13 - DNA, 721 bp, Homo sapiens
tttgggttcccctttgtgatgggtaggggaaaacaagatctgagaatttaaagagtctgagtttttcttctttctctcctcccacagctcgtccgtagattggaatcgccctgaagatgtagaccctcaagggatttatgtcatatctgctccttccatctacgctcgggaggtagcgacgccccttttccccccgctacacactgggcgcgctgggcagaggcagcacctgctttttccctacccttcctcgattctgtccgtgaaatgaattgggtagagtctctggaaggttttaagcccattttcagttctaacttactttcatcctattttgcatccctcttatcgttttgagctacctgccatcttctctttgaaaaacctatgggcttgaggaggtcacgatgccgactccgccagagcttttccactgattgtactcagcggggaggcaggggaggcagaggggcagcctctctaatgcttcctactcattttgtttctaggcctgacgcgtctcctccatccgcacctggagtcagagcgtggatttttgtatttgctcggtggtgcccagtctctgccccagaggctttggagttcaatcttgaaggggtgtctgggggaactttactgttgcaagttgtaaataatggttatttatatcctattttttctcaccccatctctctagaaacacctataaaggctattattg

SEQ ID NO: 14 - protein, 618 aa, Homo sapiens
MVSPRMSGLLSQTVILALIFLPQTRPAGVFELQIHSFGPGPGPGAPRSPCSARLPCRLFFRVCLKPGLSEEAAESPCALGAALSARGPVYTEQPGAPAPDLPLPDGLLQVPFRDAWPGTFSFIIETWREELGDQIGGPAWSLLARVAGRRRLAAGGPWARDIQRAGAWELRFSYRARCEPPAVGTACTRLCRPRSAPSRCGPGLRPCAPLEDECEAPLVCRAGCSPEHGFCEQPGECRCLEGWTGPLCTVPVSTSSCLSPRGPSSATTGCLVPGPGPCDGNPCANGGSCSETPRSFECTCPRGFYGLRCEVSGVTCADGPCFNGGLCVGGADPDSAYICHCPPGFQGSNCEKRVDRCSLQPCRNGGLCLDLGHALRCRCRAGFAGPRCEHDLDDCAGRACANGGTCVEGGGAHRCSCALGFGGRDCRERADPCAARPCAHGGRCYAHFSGLVCACAPGYMGARCEFPVHPDGASALPAAPPGLRPGDPQRYLLPPALGLLVAAGVAGAALLLVHVRRRGHSQDAGSRLLAGTPEPSVHALPDALNNLRTQEGSGDGPSSSVDWNRPEDVDPQGIYVISAPSIYAREVATPLFPPLHTGRAGQRQHLLFPYPSSILSVK

SEQ ID NO: 15 - DNA, 2058 bp, Homo sapiens
atggcggcagcgtcccggagcgcctctggctgggcgctactgctgctggtggcactttggcagcagcgcgcggccggctccggcgtcttccagctgcagctgcaggagttcatcaacgagcgcggcgtactggccagtgggcggccttgcgagcccggctgccggactttcttccgcgtctgccttaagcacttccaggcggtcgtctcgcccggaccctgcaccttcgggaccgtctccacgccggtattgggcaccaactccttcgctgtccgggacgacagtagcggcggggggcgcaaccctctccaactgcccttcaatttcacctggccgggtaccttctcgctcatcatcgaagcttggcacgcgccaggagacgacctgcggccagaggccttgccaccagatgcactcatcagcaagatcgccatccagggctccctagctgtgggtcagaactggttattggatgagcaaaccagcaccctcacaaggctgcgctactcttaccgggtcatctgcagtgacaactactatggagacaactgctcccgcctgtgcaagaagcgcaatgaccacttcggccactatgtgtgccagccagatggcaacttgtcctgcctgcccggttggactggggaatattgccaacagcctatctgtctttcgggctgtcatgaacagaatggctactgcagcaagccagcagagtgcctctgccgcccaggctggcagggccggctgtgtaacgaatgcatcccccacaatggctgtcgccacggcacctgcagcactccctggcaatgtacttgtgatgagggctggggaggcctgttttgtgaccaagatctcaactactgcacccaccactccccatgcaagaatggggcaacgtgctccaacagtgggcagcgaagctacacctgcacctgtcgcccaggctacactggtgtggactgtgagctggagctcagcgagtgtgacagcaacccctgtcgcaatggaggcagctgtaaggaccaggaggatggctaccactgcctgtgtcctccgggctactatggcctgcattgtgaacacagcaccttgagctgcgccgactccccctgcttcaatgggggctcctgccgggagcgcaaccagggggccaactatgcttgtgaatgtccccccaacttcaccggctccaactgcgagaagaaagtggacaggtgcaccagcaacccctgtgccaacgggggacagtgcctgaaccgaggtccaagccgcatgtgccgctgccgtcctggattcacgggcacctactgtgaactccacgtcagcgactgtgcccgtaacccttgcgcccacggtggcacttgccatgacctggagaatgggctcatgtgcacctgccctgccggcttctctggccgacgctgtgaggtgcggacatccatcgatgcctgtgcctcgagtccctgcttcaacagggccacctgctacaccgacctctccacagacacctttgtgtgcaactgcccttatggctttgtgggcagccgctgcgagttccccgtgggcttgccgcccagcttcccctgggtggccgtctcgctgggtgtggggctggcagtgctgctggtactgctgggcatggtggcagtggctgtgcggcagctgcggcttcgacggccggacgacggcagcagggaagccatgaacaacttgtcggacttccagaaggacaacctgattcctgccgcccagcttaaaaacacaaaccagaagaaggagctggaagtggactgtggcctggacaagtccaactgtggcaaacagcaaaaccacacattggactataatctggccccagggcccctggggcgggggaccatgccaggaaagtttccccacagtgacaagagcttaggagagaaggcgccactgcggttacacagtgaaaagccagagtgtcggatatcagcgatatgctcccccagggactccatgtaccagtctgtgtgtttgatatcagaggagaggaatgaatgtgtcattgccacggaggtataa

SEQ ID NO: 16 - protein, 685 aa, Homo sapiens
MAAASRSASGWALLLLVALWQQRAAGSGVFQLQLQEFINERGVLASGRPCEPGCRTFFRVCLKHFQAVVSPGPCTFGTVSTPVLGTNSFAVRDDSSGGGRNPLQLPFNFTWPGTFSLIIEAWHAPGDDLRPEALPPDALISKIAIQGSLAVGQNWLLDEQTSTLTRLRYSYRVICSDNYYGDNCSRLCKKRNDHFGHYVCQPDGNLSCLPGWTGEYCQQPICLSGCHEQNGYCSKPAECLCRPGWQGRLCNECIPHNGCRHGTCSTPWQCTCDEGWGGLFCDQDLNYCTHHSPCKNGATCSNSGQRSYTCTCRPGYTGVDCELELSECDSNPCRNGGSCKDQEDGYHCLCPPGYYGLHCEHSTLSCADSPCFNGGSCRERNQGANYACECPPNFTGSNCEKKVDRCTSNPCANGGQCLNRGPSRMCRCRPGFTGTYCELHVSDCARNPCAHGGTCHDLENGLMCTCPAGFSGRRCEVRTSIDACASSPCFNRATCYTDLSTDTFVCNCPYGFVGSRCEFPVGLPPSFPWVAVSLGVGLAVLLVLLGMVAVAVRQLRLRRPDDGSREAMNNLSDFQKDNLIPAAQLKNTNQKKELEVDCGLDKSNCGKQQNHTLDYNLAPGPLGRGTMPGKFPHSDKSLGEKAPLRLHSEKPECRISAICSPRDSMYQSVCLISEERNECVIATEV

SEQ ID NO: 17 - DNA, 5942 bp, Homo sapiens
ccgggtccttctccgagagccgggcgggcacgcgtcattgtgttacctgcggccggcccgcgagctaggctggttttttttttttctcccctccctcccccctttttccatgcagctgatctaaaagggaataaaaggctgcgcataatcataataataaaagaaggggagcgcgagagaaggaaagaaagccgggaggtggaagaggagggggagcgtctcaaagaagcgatcagaataataaaaggaggccgggctctttgccttctggaacgggccgctcttgaaagggcttttgaaaagtggtgttgttttccagtcgtgcatgctccaatcggcggagtatattagagccgggacgcggcggccgcaggggcagcggcgacggcagcaccggcggcagcaccagcgcgaacagcagcggcggcgtcccgagtgcccgcggcgcgcggcgcagcgatgcgttccccacggacgcgcggccggtccgggcgccccctaagcctcctgctcgccctgctctgtgccctgcgagccaaggtgtgtggggcctcgggtcagttcgagttggagatcctgtccatgcagaacgtgaacggggagctgcagaacgggaactgctgcggcggcgcccggaacccgggagaccgcaagtgcacccgcgacgagtgtgacacatacttcaaagtgtgcctcaaggagtatcagtcccgcgtcacggccggggggccctgcagcttcggctcagggtccacgcctgtcatcgggggcaacaccttcaacctcaaggccagccgcggcaacgaccgcaaccgcatcgtgctgcctttcagtttcgcctggccgaggtcctatacgttgcttgtggaggcgtgggattccagtaatgacaccgttcaacctgacagtattattgaaaaggcttctcactcgggcatgatcaaccccagccggcagtggcagacgctgaagcagaacacgggcgttgcccactttgagtatcagatccgcgtgacctgtgatgactactactatggctttggctgcaataagttctgccgccccagagatgacttctttggacactatgcctgtgaccagaatggcaacaaaacttgcatggaaggctggatgggccgcgaatgtaacagagctatttgccgacaaggctgcagtcctaagcatgggtcttgcaaactcccaggtgactgcaggtgccagtacggctggcaaggcctgtactgtgataagtgcatcccacacccgggatgcgtccacggcatctgtaatgagccctggcagtgcctctgtgagaccaactggggcggccagctctgtgacaaagatctcaattactgtgggactcatcagccgtgtctcaacgggggaacttgtagcaacacaggccctgacaaatatcagtgttcctgccctgaggggtattcaggacccaactgtgaaattgctgagcacgcctgcctctctgatccctgtcacaacagaggcagctgtaaggagacctccctgggctttgagtgtgagtgttccccaggctggaccggccccacatgctctacaaacattgatgactgttctcctaataactgttcccacgggggcacctgccaggacctggttaacggatttaagtgtgtgtgccccccacagtggactgggaaaacgtgccagttagatgcaaatgaatgtgaggccaaaccttgtgtaaacgccaaatcctgtaagaatctcattgccagctactactgcgactgtcttcccggctggatgggtcagaattgtgacataaatattaatgactgccttggccagtgtcagaatgacgcctcctgtcgggatttggttaatggttatcgctgtatctgtccacctggctatgcaggcgatcactgtgagagagacatcgatgaatgtgccagcaacccctgtttggatgggggtcactgtcagaatgaaatcaacagattccagtgtctgtgtcccactggtttctctggaaacctctgtcagctggacatcgattattgtgagcctaatccctgccagaacggtgcccagtgctacaaccgtgccagtgactatttctgcaagtgccccgaggactatgagggcaagaactgctcacacctgaaagaccactgccgcacgaccccctgtgaagtgattgacagctgcacagtggccatggcttccaacgacacacctgaaggggtgcggtatatttcctccaacgtctgtggtcctcacgggaagtgcaagagtcagtcgggaggcaaattcacctgtgactgtaacaaaggcttcacgggaacatactgccatgaaaatattaatgactgtgagagcaacccttgtagaaacggtggcacttgcatcgatggtgtcaactcctacaagtgcatctgtagtgacggctgggagggggcctactgtgaaaccaatattaatgactgcagccagaacccctgccacaatgggggcacgtgtcgcgacctggtcaatgacttctactgtgactgtaaaaatgggtggaaaggaaagacctgccactcacgtgacagtcagtgtgatgaggccacgtgcaacaacggtggcacctgctatgatgagggggatgcttttaagtgcatgtgtcctggcggctgggaaggaacaacctgtaacatagcccgaaacagtagctgcctgcccaacccctgccataatgggggcacatgtgtggtcaacggcgagtcctttacgtgcgtctgcaaggaaggctgggaggggcccatctgtgctcagaataccaatgactgcagccctcatccctgttacaacagcggcacctgtgtggatggagacaactggtaccggtgcgaatgtgccccgggttttgctgggcccgactgcagaataaacatcaatgaatgccagtcttcaccttgtgcctttggagcgacctgtgtggatgagatcaatggctaccggtgtgtctgccctccagggcacagtggtgccaagtgccaggaagtttcagggagaccttgcatcaccatggggagtgtgataccagatggggccaaatgggatgatgactgtaatacctgccagtgcctgaatggacggatcgcctgctcaaaggtctggtgtggccctcgaccttgcctgctccacaaagggcacagcgagtgccccagcgggcagagctgcatccccatcctggacgaccagtgcttcgtccacccctgcactggtgtgggcgagtgtcggtcttccagtctccagccggtgaagacaaagtgcacctctgactcctattaccaggataactgtgcgaacatcacatttacctttaacaaggagatgatgtcaccaggtcttactacggagcacatttgcagtgaattgaggaatttgaatattttgaagaatgtttccgctgaatattcaatctacatcgcttgcgagccttccccttcagcgaacaatgaaatacatgtggccatttctgctgaagatatacgggatgatgggaacccgatcaaggaaatcactgacaaaataattgatcttgttagtaaacgtgatggaaacagctcgctgattgctgccgttgcagaagtaagagttcagaggcggcctctgaagaacagaacagatttccttgttcccttgctgagctctgtcttaactgtggcttggatctgttgcttggtgacggccttctactggtgcctgcggaagcggcggaagccgggcagccacacacactcagcctctgaggacaacaccaccaacaacgtgcgggagcagctgaaccagatcaaaaaccccattgagaaacatggggccaacacggtccccatcaaggattacgagaacaagaactccaaaatgtctaaaataaggacacacaattctgaagtagaagaggacgacatggacaaacaccagcagaaagcccggtttgccaagcagccggcgtatacgctggtagacagagaagagaagccccccaacggcacgccgacaaaacacccaaactggacaaacaaacaggacaacagagacttggaaagtgcccagagcttaaaccgaatggagtacatcgtatagcagaccgcgggcactgccgccgctaggtagagtctgagggcttgtagttctttaaactgtcgtgtcatactcgagtctgaggccgttgctgacttagaatccctgtgttaatttaagttttgacaagctggcttacactggcaatggtagtttctgtggttggctgggaaatcgagtgccgcatctcacagctatgcaaaaagctagtcaacagtaccctggttgtgtgtccccttgcagccgacacggtctcggatcaggctcccaggagcctgcccagccccctggtctttgagctcccacttctgccagatgtcctaatggtgatgcagtcttagatcatagttttatttatatttattgactcttgagttgtttttgtatattggttttatgatgacgtacaagtagttctgtatttgaaagtgcctttgcagctcagaaccacagcaacgatcacaaatgactttattatttatttttttaattgtatttttgttgttgggggaggggagactttgatgtcagcagttgctggtaaaatgaagaatttaaagaaaaaaatgtcaaaagtagaactttgtatagttatgtaaataattcttttttattaatcactgtgtatatttgatttattaacttaataatcaagagccttaaaacatcattcctttttatttatatgtatgtgtttagaattgaaggtttttgatagcattgtaagcgtatggctttatttttttgaactcttctcattacttgttgcctataagccaaaattaaggtgtttgaaaatagtttattttaaaacaataggatgggcttctgtgcccagaatactgatggaattttttttgtacgacgtcagatgtttaaaacaccttctatagcatcacttaaaacacgttttaaggactgactgaggcagtttgaggattagtttagaacaggtttttttgtttgtttgttttttgtttttctgctttagacttgaaaagagacaggcaggtgatctgctgcagagcagtaagggaacaagttgagctatgacttaacatagccaaaatgtgagtggttgaatatgattaaaaatatcaaattaattgtgtgaacttggaagcacaccaatctgactttgtaaattctgatttcttttcaccattcgtacataatactgaaccacttgtagatttgattttttttttaatctactgcatttagggagtattctaataagctagttgaatacttgaaccataaaatgtccagtaagatcactgtttagatttgccatagagtacactgcctgccttaagtgaggaaatcaaagtgctattacgaagttcaagatcaaaaaggcttataaaacagagtaatcttgttggttcaccattgagaccgtgaagatactttgtattgtcctattagtgttatatgaacatacaaatgcatctttgatgtgttgttcttggcaataaattttgaaaagtaatatttattaaatttttttgtatgaaaacatggaacagtgtggctcttctgagcttacgtagttctaccggctttgccgtgtgcttctgccaccctgctgagtctgttctggtaatcggggtataataggctctgcctgacagagggatggaggaagaactgaaaggcttttcaaccacaaaactcatctggagttctcaaagacctggggctgctgtgaagctggaactgcgggagccccatctaggggagccttgattcccttgttattcaacagcaagtgtgaatactgcttgaataaacaccactggattaatggaaaaaaaaaaaaaaaa

SEQ ID NO: 18 - protein, 1218 aa, Homo sapiens
MRSPRTRGRSGRPLSLLLALLCALRAKVCGASGQFELEILSMQNVNGELQNGNCCGGARNPGDRKCTRDECDTYFKVCLKEYQSRVTAGGPCSFGSGSTPVIGGNTFNLKASRGNDRNRIVLPFSFAWPRSYTLLVEAWDSSNDTVQPDSIIEKASHSGMINPSRQWQTLKQNTGVAHFEYQIRVTCDDYYYGFGCNKFCRPRDDFFGHYACDQNGNKTCMEGWMGPECNRAICRQGCSPKHGSCKLPGDCRCQYGWQGLYCDKCIPHPGCVHGICNEPWQCLCETNWGGQLCDKDLNYCGTHQPCLNGGTCSNTGPDKYQCSCPEGYSGPNCEIAEHACLSDPCHNRGSCKETSLGFECECSPGWTGPTCSTNIDDCSPNNCSHGGTCQDLVNGFKCVCPPQWTGKTCQLDANECEAKPCVNAKSCKNLIASYYCDCLPGWMGQNCDININDCLGQCQNDASCRDLVNGYRCICPPGYAGDHCERDIDECASNPCLNGGHCQNEINRFQCLCPTGFSGNLCQLDIDYCEPNPCQNGAQCYNRASDYFCKCPEDYEGKNCSHLKDHCRTTPCEVIDSCTVAMASNDTPEGVRYISSNVCGPHGKCKSQSGGKFTCDCNKGFTGTYCHENINDCESNPCRNGGTCIDGVNSYKCICSDGWEGAYCETNINDCSQNPCHNGGTCRDLVNDFYCDCKNGWKGKTCHSRDSQCDEATCNNGGTCYDEGDAFKCMCPGGWEGTTCNIARNSSCLPNPCHNGGTCVVNGESFTCVCKEGWEGPICAQNTNDCSPHPCYNSGTCVDGDNWYRCECAPGFAGPDCRININECQSSPCAFGATCVDEINGYRCVCPPGHSGAKCQEVSGRPCITMGSVIPDGAKWDDDCNTCQCLNGRIACSKVWCGPRPCLLHKGHSECPSGQSCIPILDDQCFVHPCTGVGECRSSSLQPVKTKCTSDSYYQDNCANITFTFNKEMMSPGLTTEHICSELRNLNILKNVSAEYSIYIACEPSPSANNEIHVAISAEDIRDDGNPIKEITDKIIDLVSKRDGNSSLIAAVAEVRVQRRPLKNRTDFLVPLLSSVLTVAWICCLVTAFYWCLRKRRKPGSHTHSASEDNTTNNVREQLNQIKNPIEKHGANTVPIKDYENKNSKMSKIRTHNSEVEEDDMDKHQQKARFAKQPAYTLVDREEKPPNGTPTKHPNWTNKQDNRDLESAQSLNRMEYIV

SEQ ID NO: 19 - DNA, 4749 bp, Homo sapiens
ggagcgggcgcgcggcggcggcggggccgcggcgggcgggtcgcgggggcaatgcgggcgcagggccggggggccttccccccggcgctgctgctgctgctggcgctctgggtgcaggcggcgcggcccatgggctatttcgagctgcagctgagcgcgctgcggaacgtgaacggggagctgctgagcggcgcctgctgtgacggcgacggccggacaacgcgcgcggggggctgcggccacgacgagtgcgacacgtacgtgcgcgtgtgccttaaggagtaccaggccaaggtgacgcccacggggccctgcagctacggccacggcgccacgcccgtgctgggcggcaactccttctacctgccgccggcgggcgctgcgggggaccgagcgcgcgcgcggccccgggccggcggcgaccaggacccgggcttcgtcgtcatccccttccagttcgcctggccgcgctcctttaccctcatcgtggaggcctgggactgggacaacgataccaccccgaatgaggagctgctgatcgagcgagtgtcgcatgccggcatgatcaacccggaggaccgctggaagagcctgcacttcagcggccacgtggcgcacctggagctgcagatccgcgtgcgctgcgacgagaactactacagcgccacttgcaacaagttctgccggccccgcaacgactttttcggccactacacctgcgaccagtacggcaacaaggcctgcatggacggctggatgggcaaggagtgcaaggaagctgtgtgtaaacaagggtgtaatttgctccacgggggatgcaccgtgcctggggagtgcaggtgcagctacggctggcaagggaggttctgcgatgagtgtgtcccctaccccggctgcgtgcatggcagttgtgtggagccctggcagtgcaactgtgagaccaactggggcggcctgctctgtgacaaagacctgaactactgtggcagccaccacccctgcaccaacggaggcacgtgcatcaacgccgagcctgaccagtaccgctgcacctgccctgacggctactcgggcaggaactgtgagaaggctgagcacgcctgcacctccaacccgtgtgccaacgggggctcttgccatgaggtgccgtccggcttcgaatgccactgcccatcgggctggagcgggcccacctgtgcccttgacatcgatgagtgtgcttcgaacccgtgtgcggccggtggcacctgtgtggaccaggtggacggctttgagtgcatctgccccgagcagtgggtgggggccacctgccagctggacgtcaacgactgtgaagggaagccatgccttaacgctttttcttgcaaaaacctgattggcggctattactgtgattgcatcccgggctggaagggcatcaactgccatatcaacgtcaacgactgtcgcgggcagtgtcagcatgggggcacctgcaaggacctggtgaacgggtaccagtgtgtgtgcccacggggcttcggaggccggcattgcgagctggaacgagacaagtgtgccagcagcccctgccacagcggcggcctctgcgaggacctggccgacggcttccactgccactgcccccagggcttctccgggcctctctgtgaggtggatgtcgacctttgtgagccaagcccctgccggaacggcgctcgctgctataacctggagggtgactattactgcgcctgccctgatgactttggtggcaagaactgctccgtgccccgcgagccgtgccctggcggggcctgcagagtgatcgatggctgcgggtcagacgcggggcctgggatgcctggcacagcagcctccggcgtgtgtggcccccatggacgctgcgtcagccagccagggggcaacttttcctgcatctgtgacagtggctttactggcacctactgccatgagaacattgacgactgcctgggccagccctgccgcaatgggggcacatgcatcgatgaggtggacgccttccgctgcttctgccccagcggctgggagggcgagctctgcgacaccaatcccaacgactgccttcccgatccctgccacagccgcggccgctgctacgacctggtcaatgacttctactgtgcgtgcgacgacggctggaagggcaagacctgccactcacgcgagttccagtgcgatgcctacacctgcagcaacggtggcacctgctacgacagcggcgacaccttccgctgcgcctgcccccccggctggaagggcagcacctgcgccgtcgccaagaacagcagctgcctgcccaacccctgtgtgaatggtggcacctgcgtgggcagcggggcctccttctcctgcatctgccgggacggctgggagggtcgtacttgcactcacaataccaacgactgcaaccctctgccttgctacaatggtggcatctgtgttgacggcgtcaactggttccgctgcgagtgtgcacctggcttcgcggggcctgactgccgcatcaacatcgacgagtgccagtcctcgccctgtgcctacggggccacgtgtgtggatgagatcaacgggtatcgctgtagctgcccacccggccgagccggcccccggtgccaggaagtgatcgggttcgggagatcctgctggtcccggggcactccgttcccacacggaagctcctgggtggaagactgcaacagctgccgctgcctggatggccgccgtgactgcagcaaggtgtggtgcggatggaagccttgtctgctggccggccagcccgaggccctgagcgcccagtgcccactggggcaaaggtgcctggagaaggccccaggccagtgtctgcgaccaccctgtgaggcctggggggagtgcggcgcagaagagccaccgagcaccccctgcctgccacgctccggccacctggacaataactgtgcccgcctcaccttgcatttcaaccgtgaccacgtgccccagggcaccacggtgggcgccatttgctccgggatccgctccctgccagccacaagggctgtggcacgggaccgcctgctggtgttgctttgcgaccgggcgtcctcgggggccagtgccgtggaggtggccgtgtccttcagccctgccagggacctgcctgacagcagcctgatccagggcgcggcccacgccatcgtggccgccatcacccagcgggggaacagctcactgctcctggctgtcaccgaggtcaaggtggagacggttgttacgggcggctcttccacaggtctgctggtgcctgtgctgtgtggtgccttcagcgtgctgtggctggcgtgcgtggtcctgtgcgtgtggtggacacgcaagcgcaggaaagagcgggagaggagccggctgccgcgggaggagagcgccaacaaccagtgggccccgctcaaccccatccgcaaccccatcgagcggccggggggccacaaggacgtgctctaccagtgcaagaacttcacgccgccgccgcgcagggcggacgaggcgctgcccgggccggccggccacgcggccgtcagggaggatgaggaggacgaggatctgggccgcggtgaggaggactccctggaggcggagaagttcctctcacacaaattcaccaaagatcctggccgctcgccggggaggccggcccactgggcctcaggccccaaagtggacaaccgcgcggtcaggagcatcaatgaggcccgctacgccggcaaggagtaggggcggctgcagctgggccgggacccagggccctcggtgggagccatgccgtctgccggacccggagccgaggcatgtgcatagtttctttattttgtgtaaaaaaaccaccaaaaacaaaaaccaaatgtttattttctacgtttctttaaccttgtataaattattcagtaactgtcaggctgaaaacaatggagtattctcggatagttgctatttttgtaaagtttccgtgcgtggcactcgctgtatgaaaggagagagcaaagggtgtctgcgtcgtcaccaaatcgtagcgtttgttaccagaggttgtgcactgtttacagaatcttccttttattcctcactcgggtttctctgtggctccaggccaaagtgccggtgagacccatggctgtgttggtgtggcccatggctgttggtgggacccgtggctgatggtgtggcctgtggctgtcggtgggactcgtggctgtcaatgggacctgtggctgtcggtgggacctacggtggtcggtgggaccctggttattgatgtggccctggctgccggcacggcccgtggctgttgacgcacctgtggttgttagtggggcctgaggtcatcggcgtgcccaaggccggcaggtcaacctcgcgcttgctggccagtccaccctgcctgccgtctgtgcttcctcctgcccagaacgcccgctccagcgatctctccactgtgctttcagaagtgcccttcctgctgcgcagttctcccatcctgggacggcggcagtattgaagctcgtgacaagtgccttcacacagacccctcgcaactgtccacgcgtgccgtggcaccaggcgctgcccacctgccggccccggccgcccctcctcgtgaaagtgcatttttgtaaatgtgtacatattaaaggaagcactctgtatatttgattgaataatgccaccaaaaaaaaaaaaaaaaaaaaattcctgccc

SEQ ID NO: 20 - protein, 1238 aa, Homo sapiens
MRAQGRGRLPRRLLLLLALWVQAARPMGYFELQLSALRNVNGELLSGACCDGDGRTTRAGGCGHDECDTYVRVCLKEYQAKVTPTGPCSYGHGATPVLGGNSFYLPPAGAAGDRARARARAGGDQDPGLVVIPFQFAWPRSFTLIVEAWDWDNDTTPNEELLIERVSHAGMINPEDRWKSLHFSGHVAHLELQIRVRCDENYYSATCNKFCRPRNDFFGHYTCDQYGNKACMDGWMGKECKEAVCKQGCNLLHGGCTVPGECRCSYGWQGRFCDECVPYPGCVHGSCVEPWQCNCETNWGGLLCDKDLNYCGSHHPCTNGGTCINAEPDQYRCTCPDGYSGRNCEKAEHACTSNPCANGGSCHEVPSGFECHCPSGWSGPTCALDIDECASNPCAAGGTCVDQVDGFECICPEQWVGATCQLDANECEGKPCLNAFSCKNLIGGYYCDCIPGWKGINCHINVNDCRGQCQHGGTCKDLVNGYQCVCPRGFGGRHCELERDECASSPCHSGGLCEDLADGFHCHCPQGFSGPLCEVDVDLCEPSPCRNGARCYNLEGDYYCACPDDFGGKNCSVPREPCPGGACRVIDGCGSDAGPGMPGTAASGVCGPHGRCVSQPGGNFSCICDSGFTGTYCHENIDDCLGQPCRNGGTCIDEVDAFRCFCPSGWEGELCDTNPNDCLPDPCHSRGRCYDLVNDFYCACDDGWKGKTCHSREFQCDAYTCSNGGTCYDSGDTFRCACPPGWKGSTCAVAKNSSCLPNPCVNGGTCVGSGASFSCICRDGWEGRTCTHNTNDCNPLPCYNGGICVDGVNWFRCECAPGFAGPDCRINIDECQSSPCAYGATCVDEINGYRCSCPPGRAGPRCQEVIGFGRSCWSRGTPFPHGSSWVEDCNSCRCLDGRRDCSKVWCGWKPCLLAGQPEALSAQCPLGQRCLEKAPGQCLRPPCEAWGECGAEEPPSTPCLPRSGHLDNNCARLTLHFNRDHVPQGTTVGAICSGIRSLPATRAVARDRLLVLLCDRASSGASAVEVAVSFSPARDLPDSSLIQGAAHAIVAAITQRGNSSLLLAVTEVKVETVVTGGSSTGLLVPVLCGAFSVLWLACVVLCVWWTRKRRKERERSRLPREESANNQWAPLNPIRNPIERPGGHKDVLYQCKNFTPPPRRADEALPGPAGHAAVREDEEDEDLGRGEEDSLEAEKFLSHKFTKDPGRSPGRPAHWASGPKVDNRAVRSINEARYAGKE

SEQ ID NO: 21 - DNA, 2292 bp, Homo sapiens
atggagagcaaggtgctgctggccgtcgccctgtggctctgcgtggagacccgggccgcctctgtgggtttgcctagtgtttctcttgatctgcccaggctcagcatacaaaaagacatacttacaattaaggctaatacaactcttcaaattacttgcaggggacagagggacttggactggctttggcccaataatcagagtggcagtgagcaaagggtggaggtgactgagtgcagcgatggcctcttctgtaagacactcacaattccaaaagtgatcggaaatgacactggagcctacaagtgcttctaccgggaaactgacttggcctcggtcatttatgtctatgttcaagattacagatctccatttattgcttctgttagtgaccaacatggagtcgtgtacattactgagaacaaaaacaaaactgtggtgattccatgtctcgggtccatttcaaatctcaacgtgtcactttgtgcaagatacccagaaaagagatttgttcctgatggtaacagaatttcctgggacagcaagaagggctttactattcccagctacatgatcagctatgctggcatggtcttctgtgaagcaaaaattaatgatgaaagttaccagtctattatgtacatagttgtcgttgtagggtataggatttatgatgtggttctgagtccgtctcatggaattgaactatctgttggagaaaagcttgtcttaaattgtacagcaagaactgaactaaatgtggggattgacttcaactgggaatacccttcttcgaagcatcagcataagaaacttgtaaaccgagacctaaaaacccagtctgggagtgagatgaagaaatttttgagcaccttaactatagatggtgtaacccggagtgaccaaggattgtacacctgtgcagcatccagtgggctgatgaccaagaagaacagcacatttgtcagggtccatgaaaaaccttttgttgcttttggaagtggcatggaatctctggtggaagccacggtgggggagcgtgtcagaatccctgcgaagtaccttggttacccacccccagaaataaaatggtataaaaatggaataccccttgagtccaatcacacaattaaagcggggcatgtactgacgattatggaagtgagtgaaagagacacaggaaattacactgtcatccttaccaatcccatttcaaaggagaagcagagccatgtggtctctctggttgtgtatgtcccaccccagattggtgagaaatctctaatctctcctgtggattcctaccagtacggcaccactcaaacgctgacatgtacggtctatgccattcctcccccgcatcacatccactggtattggcagttggaggaagagtgcgccaacgagcccagccaagctgtctcagtgacaaacccatacccttgtgaagaatggagaagtgtggaggacttccagggaggaaataaaattgaagttaataaaaatcaatttgctctaattgaaggaaaaaacaaaactgtaagtacccttgttatccaagcggcaaatgtgtcagctttgtacaaatgtgaagcggtcaacaaagtcgggagaggagagagggtgatctccttccacgtgaccaggggtcctgaaattactttgcaacctgacatgcagcccactgagcaggagagcgtgtctttgtggtgcactgcagacagatctacgtttgagaacctcacatggtacaagcttggcccacagcctctgccaatccatgtgggagagttgcccacacctgtttgcaagaacttggatactctttggaaattgaatgccaccatgttctctaatagcacaaatgacattttgatcatggagcttaagaatgcatccttgcaggaccaaggagactatgtctgccttgctcaagacaggaagaccaagaaaagacattgcgtggtcaggcagctcacagtcctagagcgtgtggcacccacgatcacaggaaacctggagaatcagacgacaagtattggggaaagcatcgaagtctcatgcacggcatctgggaatccccctccacagatcatgtggtttaaagataatgagacccttgtagaagactcaggcattgtattgaaggatgggaaccggaacctcactatccgcagagtgaggaaggaggacgaaggcctctacacctgccaggcatgcagtgttcttggctgtgcaaaagtggaggcatttttcataatagaaggtgcccaggaaaagacgaacttggaa

SEQ ID NO: 22 - protein, 764 aa, Homo sapiens
MESKVLLAVALWLCVETRAASVGLPSVSLDLPRLSIQKDILTIKANTTLQITCRGQRDLDWLWPNNQSGSEQRVEVTECSDGLFCKTLTIPKVIGNDTGAYKCFYRETDLASVIYVYVQDYRSPFIASVSDQHGVVYITENKNKTVVIPCLGSISNLNVSLCARYPEKRFVPDGNRISWDSKKGFTIPSYMISYAGMVFCEAKINDESYQSIMYIVVVVGYRIYDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEKPFVAFGSGMESLVEATVGERVRIPAKYLGYPPPEIKWYKNGIPLESNHTIKAGHVLTIMEVSERDTGNYTVILTNPISKEKQSHVVSLVVYVPPQIGEKSLISPVDSYQYGTTQTLTCTVYAIPPPHHIHWYWQLEEECANEPSQAVSVTNPYPCEEWRSVEDFQGGNKIEVNKNQFALIEGKNKTVSTLVIQAANVSALYKCEAVNKVGRGERVISFHVTRGPEITLQPDMQPTEQESVSLWCTADRSTFENLTWYKLGPQPLPIHVGELPTPVCKNLDTLWKLNATMFSNSTNDILIMELKNASLQDQGDYVCLAQDRKTKKRHCVVRQLTVLERVAPTITGNLENQTTSIGESIEVSCTASGNPPPQIMWFKDNETLVEDSGIVLKDGNRNLTIRRVRKEDEGLYTCQACSVLGCAKVEAFFIIEGAQEKTNLE

SEQ ID NO: 23 - DNA, 25 bp, Artificial Sequence
  Synthetic primer
ttgttgttaacttgtttattgcagc

SEQ ID NO: 24 - DNA, 21 bp, Artificial Sequence
  Synthetic primer
tgagtttggacaaaccacaac